(19) United States
(12) Patent Application Publication
(10) Pub. No.: US 2025/0243208 A1
(43) Pub. Date: Jul. 31, 2025

(54) BICYCLIC HETEROCYCLES AS MRGPRX2 ANTAGONISTS

(71) Applicant: Incyte Corporation, Wilmington, DE (US)

(72) Inventors: Oleg Vechorkin, Wilmington, DE (US); Heeoon Han, Elkins Park, PA (US); Hunter Sims, Wilmington, DE (US)

(21) Appl. No.: 19/036,820

(22) Filed: Jan. 24, 2025

Related U.S. Application Data
(60) Provisional application No. 63/655,871, filed on Jun. 4, 2024.
(60) Provisional application No. 63/625,041, filed on Jan. 25, 2024.

(51) Int. Cl. C07D 487/04; A61K 31/519; A61K 31/537; C07D 519/00

(52) U.S. Cl. CPC C07D 487/04; A61K 31/519; A61K 31/537; C07D 519/00

(57) ABSTRACT

The present disclosure relates to bicyclic heterocycles of Formula (I), and pharmaceutical compositions of the same, that are modulators, antagonists, or inhibitors of the G protein-coupled receptor MRGPRX2 and are useful in the treatment of MRGPRX2 dependent conditions such as inflammatory diseases.

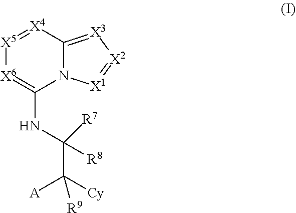

### CROSS-REFERENCE TO RELATED APPLICATIONS

This application claims the benefit of priority to U.S. Provisional Application No. 63/655,871 filed on Jun. 4, 2024 and U.S. Provisional Application No. 63/625,041 filed on Jan. 25, 2024, the contents of each of which are hereby incorporated by reference.

### FIELD

The present disclosure relates to bicyclic heterocycles, and pharmaceutical compositions of the same, that are modulators, antagonists or inhibitors of the G protein-coupled receptor MRGPRX2 and are useful in the treatment of MRGPRX2 dependent conditions such as inflammatory diseases.

### BACKGROUND

Mas-related G protein-coupled receptor X2 (MRGPRX2) is an orphan, seven transmembrane G protein-coupled receptor that is almost exclusively expressed on connective tissue mast cells. MRGPRX2 belongs to a G protein-coupled receptor subfamily X, comprised of four members X1-X4, specific to humans and primates. MRGPRX2 is a low affinity promiscuous receptor for cyclic and polybasic structure ligands that mediates mast cell degranulation in response to multiple endogenous and exogenous stimuli.

Mast cells constitute an integral part of the human immune system. They are important modulators of inflammatory and physiological processes. MRGPRX2 receptor plays a pivotal role in itch, allergy and inflammation. Activation of the receptor by neuropeptides, antimicrobial host defense peptides as well as numerous FDA-approved drugs leads to mast cell degranulation and release of inflammatory mediators through immunoglobulin-independent pathway (M. Thapaliya, et al., Curr. Allergy Asthma Rep., 2021, 21(1), 3).

Activation of MRGPRX2 receptor drives non-histaminergic itch in chronic refractory pruritus and MRGPRX2 has also been implicated in senile itch (A. He, et al. Biomed. Res. Int., 2017, 4790810; J. Meixiong, et al., Immunity, 2019, 50(5), 1163-71). There is an increased MRGPRX2 gene expression on mast cells in the skin of patients with severe chronic urticaria (hives) (H. Ali, J. Immunobiol., 2016, 1(4), 115; D. Fujisawa, et al., J. Allergy Clin. Immunol. 2014, 134(3), 622-33). Additionally, activation of MRGPRX2 by elevated levels of proadrenomedullin N-terminal 20 peptide (PAMP1-20) in the skin of patients with allergic contact dermatitis (ACD) leads to intensely itchy eczematous skin rash (J. Meixiong, et al., Immunity, 2019, 50(5):1163-71). MRGPRX2 is also involved in the pathogenesis of acne rosacea where dysregulation of the host defense mechanism due to excessive LL-37 antimicrobial peptide production leads to enhanced mast cell activation through MRGPRX2 (H. Ali, Adv. Immunol., 2017, 136, 123-62). MRGPRX2 is also implicated in systemic mastocytosis and in neurogenic inflammation, pain and itch. Substance P released from nerve endings and directly from mast cells in sickle cell anemia patients activates mast cells via MRGPRX2 causing painful crisis (H. Subramanian, et al., J. Allergy Clin. Immunol., 2016, 138(3), 700-10).

The role of MRGPRX2 in mast cell biology is further supported by the fact that naturally occurring missense MRGPRX2 variants: G165E, D184H, W243R, and H259Y inhibit mast cell degranulation in response to endogenous neuropeptides and drugs (I. Alkanfari, et al., J. Immuol., 2018, 201(2), 343-49).

Taken together, these findings suggest that MRGPRX2 plays a critical role in itch, pain, and inflammation. Potential disease indications for MRGPRX2 antagonist encompass chronic urticaria and pruritus (hives/itch), acne rosacea, and systemic mastocytosis. These clinical indications present high unmet medical need, particularly in antihistamine-refractory patients.

Therefore, targeting of MRGPRX2 receptor can be useful in the clinical treatment of mast-cell mediated diseases.

Compounds that modulate MRGPRX2 are discussed in WO2006066599A2, WO2008052072A2, WO2020223255A1, WO2021092240A1, WO2021092262A1, WO2021092264A1, WO2022067094A1, WO2022073904A1, WO2022073905A1, WO2022087083A1, WO2022111473A1, WO2022125636A1, WO2022140520A1, WO2022152852A1, WO2022152853A1, and WO2023039448A1.

There remains a need for new compounds that are effective as modulators, antagonists or inhibitors of MRGPRX2.

### SUMMARY

The present disclosure is directed to compounds having Formula (I):

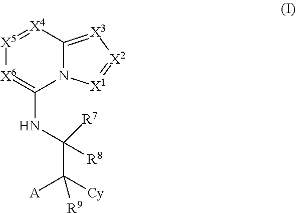

or pharmaceutically acceptable salts thereof, wherein constituent variables are defined herein.

The present disclosure is further directed to pharmaceutical compositions comprising a compound of Formula (I), or a pharmaceutically acceptable salt thereof, and at least one pharmaceutically acceptable carrier.

The present disclosure is further directed to methods of modulating such as by antagonizing or inhibiting MRGPRX2 protein comprising contacting the protein with a compound of Formula (I), or a pharmaceutically acceptable salt thereof.

The present disclosure is further directed to a method of treating MRGPRX2 dependent conditions, comprising administering a compound of Formula (I), or a pharmaceutically acceptable salt thereof, to a patient in need thereof. The present disclosure is further directed to the use of compounds of Formula (I) and pharmaceutically acceptable salts thereof in the preparation of a medicament for use in therapy. The present disclosure is further directed to compounds of Formula (I) and pharmaceutically acceptable salts thereof for use in therapy.

### DETAILED DESCRIPTION

### I. Definitions

For the terms “e.g.” and “such as,” and grammatical equivalents thereof, the phrase “and without limitation” is understood to follow unless explicitly stated otherwise.

The singular forms “a,” “an,” and “the” include plural referents unless the context clearly dictates otherwise.

The term “about” means “approximately” (e.g., plus or minus approximately 10% of the indicated value).

The terms “ambient temperature” and “room temperature” are understood in the art, and refer generally to a temperature, e.g., a reaction temperature, that is about the temperature of the room in which the reaction is carried out, e.g., a temperature from about 20° C. to about 30° C.

The following abbreviations may be used herein: aq, (aqueous); br (broad); BSA (bovine serum albumin); BTK (Bruton tyrosine kinase); CHO-K1 (Chinese hamster ovary); CTMCs (connective tissue-type mast cells); CYP (cytochrome P450); d (doublet); dd (doublet of doublets); DIPEA (N,N-diisopropylethylamine); DMEM (Dulbecco's Modified Eagle Medium); DMSO (dimethylsulfoxide); EDTA (ethylenediaminetetraacetic acid); Et (ethyl); EtOAc (ethyl acetate); Et₂O (diethyl ether); EtOH (ethanol); FBS (fetal bovine serum); FCC (flash column chromatography); FcεR1 (high-affinity IgE receptor); FLIPR (Fluorescence Imaging Plate Reader); g (gram(s)); h (hour(s)); GPCR (G protein-coupled receptor); HBSS (Hanks' Balanced Salt Solution); HCl (hydrochloric acid); HEPES (N-2-hydroxyethylpiperazine-N′-2-ethanesulfonic acid); Hex (hexanes); HLM (human liver microsome); HOAc (acetic acid); HPLC (high performance liquid chromatography); hsMC (human skin mast cells); HTRF (Homogeneous Time Resolved Fluorescence); Hz (hertz); IgE (immunoglobulin E); IP1 (myo-Inositol 1 phosphate); IV (intravenous); J (coupling constant); JAK (janus kinase); KOtBu (potassium t-butoxide); LCMS (liquid chromatography-mass spectrometry); m (multiplet); M (molar); Me (methyl); MeCN (acetonitrile); MeOH (methanol); mm (millimeter(s)); MS (Mass spectrometry); NaBH₄ (sodium borohydride); NADPH (nicotinamide adenine dinucleotide phosphate); NaHCO₃ (sodium bicarbonate); NaHMDS (sodium hexamethyldisilazide); Na₂SO₄ (sodium sulfate); NFAT (Nuclear factor of activated T cells); PBS (phosphate-buffered saline); Ph (phenyl); PK (pharmacokinetic); PO (oral); prep. (preparative); RFU (Relative Fluorescence Unit); rpm (revolutions per minute); RP-HPLC (reverse-phase high performance liquid chromatography); R.T. (retention time); r.t. (room temperature); s (singlet or second(s)); sat. (saturated); SCF (stem cell factor); SFC (super-critical fluid chromatography); t (triplet or tertiary); TDI (time dependent inhibition); TEER (transepithelial electrical resistance); tert (tertiary); TLC (thin layer chromatography); tt (triplet of triplets); t-Bu (tert-butyl); Tf₂O (trifluoromethanesulfonic anhydride); TFA (trifluoroacetic acid); v/v (volume per volume); wt % (weight percent); w/v (weight in volume), μg (microgram(s)); μL (microliter(s)); μm (micrometer(s)).

Additional definitions are provided elsewhere in the present disclosure.

### II. Compounds

The present disclosure provides a compound having Formula (I):

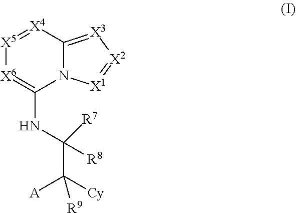

or a pharmaceutically acceptable salt thereof, wherein:
   X¹ is N or CR¹;
   X² is N or CR²;
   X³ is N or CR³;
   X⁴ is N or CR⁴;
   X⁵ is N or CR⁵;
   X⁶ is N or CR⁶;
   R¹, R², R³, R⁴, R⁵, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1}, NR^{c1}C(═NR^{c1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹, R², R³, R⁴, R⁵, and R⁶ are each optionally substituted with 1, 2, 3, or 4 substituents each independently selected from R^{10A}; and wherein the C₁₋₆ alkyl, C₂₋₆ alkenyl, and C₂₋₆ alkynyl forming R¹, R², R³, R⁴, R⁵, and R⁶ are each optionally substituted with 1, 2, 3, or 4 substituents each independently selected from R^{10B};
   R⁷ and R⁸ are each independently selected from H, D, C₁₋₆ alkyl, C₂₋₆ alkenyl, C₂₋₆ alkynyl, C₁₋₆ haloalkyl, halo, C₃₋₁₀ cycloalkyl, and 4-10 membered heterocycloalkyl, wherein the C₃₋₁₀ cycloalkyl and 4-10 membered heterocycloalkyl forming R⁷ and R⁸ are each optionally substituted with 1, 2, 3, or 4 substituents independently selected from R^{20A}; and wherein the C₁₋₆ alkyl, C₂₋₆ alkenyl, and C₂₋₆ alkynyl forming R⁷ and R⁸ are each optionally substituted with 1, 2, 3, or 4 substituents independently selected from R^{20B};
   R⁹ is selected from H, D, C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2} SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{c2})R^{b2}, C(═NOR^{a2})R^{b2}, C(═NR^{c2})NR^{c2}R^{d2}, NR^{c2}C(═NR^{c2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁹ are each optionally substituted with 1, 2, 3, or 4 substituents independently selected from R^{30A}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{30B};
   A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A};
   Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
   each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3} NR^{c3}C(O)NR^{c3}R^{d3}, C(═NR^{c3})R^{b3}, C(═NOR^{a3})R^{b3}, C((═NR^{c3})NR^{c3}R^{d3} NR^{c3}C(═NR^{c3})NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
   each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
   each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a3}, SR^{a3}, C(O)R^{b3}, C(O)NR^{c3}R^{d3}, C(O)OR^{a3}, NR^{c3}R^{d3} NR^{c3}C(O)R^{b3}, NR^{c3}C(O)OR^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
   each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4} NR^{c4}R^{d4} NR^{c4}C(O)R^{b4}, NR^{c4}C(O)OR^{a4}, NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR^{c4})NR^{c4}R^{d4} NR^{c4}S(O)R^{b4} NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R^{g1}; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
   each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR^{a5}C(═NR^{b5})NR^{c5}R^{d5} NR^{c5}S(O)R^{b5} NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
   each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
   each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{c6}C(O)R^{b6}NR^{c6}C(O)OR^{a6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6} S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R^{g1};
   each R^{10A} is independently selected from C₁₋₆ alkyl, C₂₋₆ alkenyl, C₂₋₆ alkynyl, C₁₋₆ haloalkyl, and R^{10B}; wherein the C₁₋₆ alkyl, C₂₋₆ alkenyl, and C₂₋₆ alkynyl forming R^{10A} are each optionally substituted with 1, 2, 3, or 4 substituents independently selected from R^{10B};
   each R^{10B} is independently selected from C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7} NR^{c7}R^{d7} NR^{c7}C(O)R^{b7} NR^{c7}C(O)OR^{a7} NR^{c7}C(O)NR^{c7}R^{d7}, C(═NR^{c7})R^{b7}, C(═NOR^{a7})R^{b7}, C(═NR^{c7})NR^{c7}R^{d7} NR^{c7}C(═NR^{c7})NR^{c7}R^{d7} NR^{c7}S(O)R^{b7} NR^{c7}S(O)₂R^{b7} NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{10B} are each optionally substituted with 1, 2, 3, or 4 substituents independently selected from R¹¹;
   each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a8}, SR^{a8}, C(O)R^{b8}, C(O)NR^{c8}R^{d8}, C(O)OR^{a8}, NR^{c8}R^{d8} NR^{c8}C(O)R^{b8}, NR^{c8}C(O)OR^{a8}, NR^{c8}S(O)R^{c8}, NR^{c8}S(O)₂R^{d8}, NR^{c8}S(O)₂NR^{c8}R^{d8}, S(O)R^{d8}, S(O)NR^{c8}R^{d8}, S(O)₂R^{b8}, and S(O)₂NR^{c8}R^{d8}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹¹ are each optionally substituted with 1, 2, 3, or 4 substituents independently selected from R^{g1}; and wherein the C₁₋₆ alkyl, C₂₋₆ alkenyl, and C₂₋₆ alkynyl forming R¹¹ are each optionally substituted with 1, 2, 3, or 4 substituents independently selected from R^{g2};
   each R^{20A} is independently selected from C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{20A} are each optionally substituted with 1, 2, 3, or 4 substituents independently selected from R^{20B};
   each R^{20B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a9}, SR^{a9}, C(O)R^{b9}, C(O)NR^{c9}R^{d9}, C(O)OR^{a9}, NR^{c9}R^{d9} NR^{c9}C(O)R^{d9} NR^{c9}C(O)OR^{a9} NR^{c9}S(O)R^{d9} NR^{c9}S(O)₂R^{d9}, NR^{c9}S(O)₂NR^{c9}R^{d9}, S(O)R^{d9}, S(O)NR^{c9}R^{d9} S(O)₂R^{b9}, and S(O)₂NR^{c9}R^{d9}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{20B} are each optionally substituted with 1, 2, 3, or 4 substituents independently selected from R^{g1};
   each R^{30A} is independently selected from C₁₋₆ alkyl, C₂₋₆ alkenyl, C₂₋₆ alkynyl, C₁₋₆ haloalkyl, and R^{30B}; wherein the C₁₋₆ alkyl, C₂₋₆ alkenyl, and C₂₋₆ alkynyl forming R^{30A} are each optionally substituted with 1, 2, 3, or 4 substituents independently selected from R^{30B};
   each R^{30B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a10}, SR^{a10}, C(O)R^{b10}, C(O)NR^{c10}R^{d10}, C(O)OR^{a10}, NR^{c10}R^{d10} NR^{c10}OC(O)R^{b1}, NR^{c10}C(O)OR^{a10}, NR^{c10}S(O)R^{b10} NR^{c10}S(O)₂R^{b10} NR^{c10}S(O)₂NR^{c10}R^{d10} S(O)R^{b10}, S(O)NR^{c10}R^{d10}, S(O)₂R^{b10}, and S(O)₂NR^{c10}R^{d10}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{30B} are each optionally substituted with 1, 2, 3, or 4 substituents independently selected from R^{g1};
   each R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10} and R^{d10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} are each optionally substituted with 1, 2, 3, or 4 substituents independently selected from R^{g2};
   or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c8} and R^{d8} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c9} and R^{d9} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R^{g1};
   each R^{b1}, R^{b2}, R^{b3}, R^{b4}, R^{b5}, R^{b6}, R^{b7}, R^{b8}, R^{b9}, and R^{b10} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b1}, R^{b2}, R^{b3}, R^{b4}, R^{b5}, R^{b6}, R^{b7}, R^{b8}, R^{b9}, and R^{b10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b1}, R^{b2}, R^{b3}, R^{b4}, R^{b5}, R^{b6}, R^{b7}, R^{b8}, R^{b9} and R^{b10} are each optionally substituted with 1, 2, 3, or 4 substituents independently selected from R^{g2};
   each R^{c1}, R^{c2}, R^{c3}, R^{c4}, R^{c5} and R^{c7} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
   each R^{g1} is independently selected from C₁₋₆ alkyl, C₂₋₆ alkenyl, C₂₋₆ alkynyl, and R^{g2}, wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}, R^{d11} NR^{c11}C(O)R^{b11}, NR^{d11}C(O)OR^{a11}, NR^{c11}S(O)R^{b11}, NR^{c11}S(O)₂R^{b11}, NR^{c11}S(O)₂NR^{d11}R^{d11}, S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c11}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a11}, R^{c11}, and R^{d11} are each optionally substituted with 1, 2, 3, or 4 substituents independently selected from R^{h2};
   each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
   each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2};
   each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
   wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
   at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group.

In some embodiments, the compound is not one of the following compounds:
N-[2-phenyl-2-(1-pyrrolidinyl)ethyl]tetrazolo[1,5-a]pyrazin-5-amine;
N-[2-(2-furanyl)-2-(1-pyrrolidinyl)ethyl]tetrazolo[1,5-a]pyrazin-5-amine;
N-[2-(5-methyl-2-furanyl)-2-(4-morpholinyl)ethyl]tetrazolo[1,5-a]pyrazin-5-amine;
N-[2-(4-methyl-1-piperidinyl)-2-(2-thienyl)ethyl][1,2,4]triazolo[1,5-a]pyrimidin-7-amine;
5-methyl-N-[2-phenyl-2-(4-piperidinyl)ethyl][1,2,4]triazolo[1,5-a]pyrimidin-7-amine;
5-methyl-N-[2-phenyl-2-(1-pyrrolidinyl)ethyl][1,2,4]triazolo[1,5-a]pyrimidin-7-amine;
N-[2-(2-furanyl)-2-(1-pyrrolidinyl)ethyl]-5-methyl[1,2,4]triazolo[1,5-a]pyrimidin-7-amine;
N-[2-(2-furanyl)-2-(1-piperidinyl)ethyl]-5-methyl[1,2,4]triazolo[1,5-a]pyrimidin-7-amine;
N-[2-(2-furanyl)-2-(4-morpholinyl)ethyl]-5-methyl[1,2,4]triazolo[1,5-a]pyrimidin-7-amine;
5-methyl-N-[2-(4-morpholinyl)-2-(2-thienyl)ethyl][1,2,4]triazolo[1,5-a]pyrimidin-7-amine;
5-methyl-N-[2-(4-morpholinyl)-2-(3-thienyl)ethyl][1,2,4]triazolo[1,5-a]pyrimidin-7-amine;
N-[2-(4-methoxyphenyl)-2-(1-pyrrolidinyl)ethyl]-5-methyl[1,2,4]triazolo[1,5-a]pyrimidin-7-amine;
5-methyl-N-[2-(5-methyl-2-furanyl)-2-(4-morpholinyl)ethyl][1,2,4]triazolo[1,5-a]pyrimidin-7-amine;
5-methyl-N-[2-(5-methyl-2-thienyl)-2-(4-morpholinyl)ethyl][1,2,4]triazolo[1,5-a]pyrimidin-7-amine;
5-methyl-N-[2-(2-methyl-4-morpholinyl)-2-(2-thienyl)ethyl][1,2,4]triazolo[1,5-a]pyrimidin-7-amine;
5-methyl-N-[2-(4-methylphenyl)-2-(4-morpholinyl)ethyl][1,2,4]triazolo[1,5-a]pyrimidin-7-amine;
5-methyl-N-[2-(3-methylphenyl)-2-(4-morpholinyl)ethyl][1,2,4]triazolo[1,5-a]pyrimidin-7-amine;
N-[2-(2-methoxyphenyl)-2-(1-pyrrolidinyl)ethyl]-5-methyl[1,2,4]triazolo[1,5-a]pyrimidin-7-amine;
N-[2-(4-methoxyphenyl)-2-(4-morpholinyl)ethyl]-5-methyl[1,2,4]triazolo[1,5-a]pyrimidin-7-amine;
N-[2-(4-fluorophenyl)-2-(4-morpholinyl)ethyl]-5-methyl[1,2,4]triazolo[1,5-a]pyrimidin-7-amine;
N-[2-(2-chlorophenyl)-2-(4-morpholinyl)ethyl]-5-methyl[1,2,4]triazolo[1,5-a]pyrimidin-7-amine;
N-[2-(4-methylphenyl)-2-(4-morpholinyl)ethyl]-5-phenyl[1,2,4]triazolo[1,5-a]pyrimidin-7-amine;
N-[2-(3,4-Dimethoxyphenyl)-2-(4-morpholinyl)ethyl]-5-methyl[1,2,4]triazolo[1,5-a]pyrimidin-7-amine;
N-[2-(2-methoxyphenyl)-2-(1-piperidinyl)ethyl]imidazo[1,2-a]pyrimidin-5-amine;
5-ethyl-N-[2-(2-furanyl)-2-(1-pyrrolidinyl)ethyl]pyrazolo[1,5-a]pyrimidin-7-amine;
5-ethyl-N-[2-(5-methyl-2-furanyl)-2-(4-morpholinyl)ethyl]pyrazolo[1,5-a]pyrimidin-7-amine;
N-[2-(4-morpholinyl)-2-(3-pyridinyl)ethyl]-5-propylpyrazolo[1,5-a]pyrimidin-7-amine;
N-[2-(2-furanyl)-2-(1-pyrrolidinyl)ethyl]-2,3,5-trimethylpyrazolo[1,5-a]pyrimidin-7-amine;
5-(1-methylethyl)-N-[2-(5-methyl-2-furanyl)-2-(4-morpholinyl)ethyl]pyrazolo[1,5-a]pyrimidin-7-amine;
5-(1-methylethyl)-N-[2-(4-morpholinyl)-2-(4-pyridinyl)ethyl]pyrazolo[1,5-a]pyrimidin-7-amine;
5-(1-methylethyl)-N-[2-(4-morpholinyl)-2-(2-pyridinyl)ethyl]pyrazolo[1,5-a]pyrimidin-7-amine;
2,5-dimethyl-N-[2-(4-morpholinyl)-2-(4-pyridinyl)ethyl]pyrazolo[1,5-a]pyrimidin-7-amine;
2,5-dimethyl-N-[2-(4-morpholinyl)-2-(2-pyridinyl)ethyl]pyrazolo[1,5-a]pyrimidin-7-amine;
2,3,5-trimethyl-N-[2-(4-morpholinyl)-2-(3-pyridinyl)ethyl]pyrazolo[1,5-a]pyrimidin-7-amine;
N-[2-(4-morpholinyl)-2-(2-thienyl)ethyl]imidazo[1,2-c]pyrimidin-5-amine.

In some embodiments, the compound has Formula (I-A):

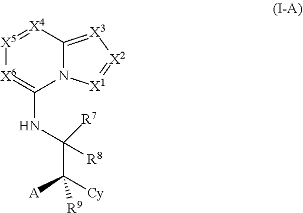

or a pharmaceutically acceptable salt thereof, wherein X¹, X², X³, X⁴, X⁵, X⁶, R⁷, R⁸, R⁹, A, and Cy are as defined herein.

In some embodiments, the compound has Formula (I-B):

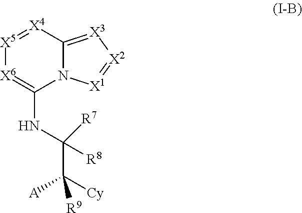

or a pharmaceutically acceptable salt thereof, wherein X¹, X², X³, X⁴, X⁵, X⁶, R⁷, R⁸, R⁹, A, and Cy are as defined herein.

In some embodiments, the compound has Formula (II-A), (II-B), (II-C), (II-D), (II-E), (II-F), (II-G), (II-I), (II-I), (II-J), (II-K), or (II-L):

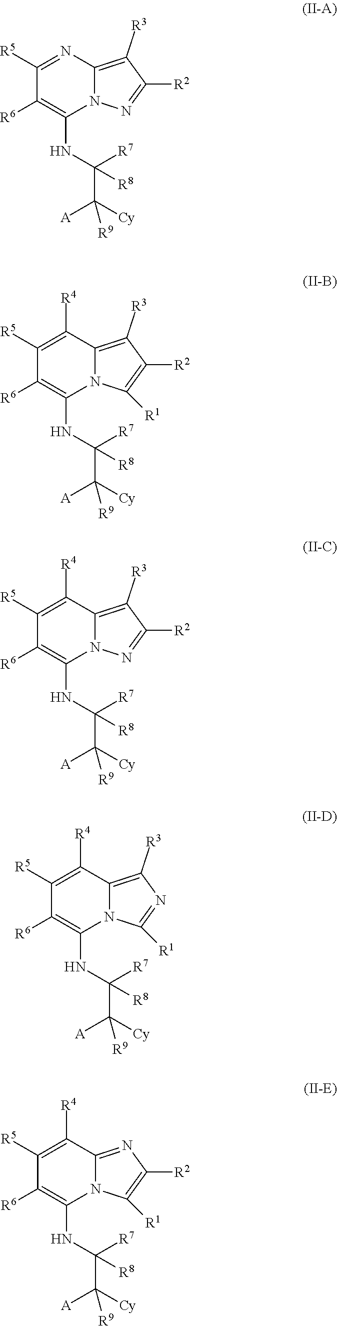

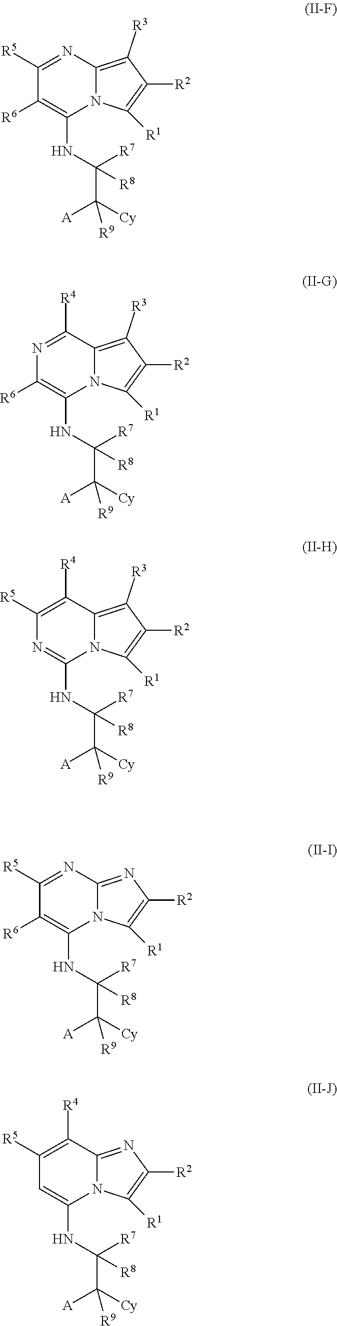

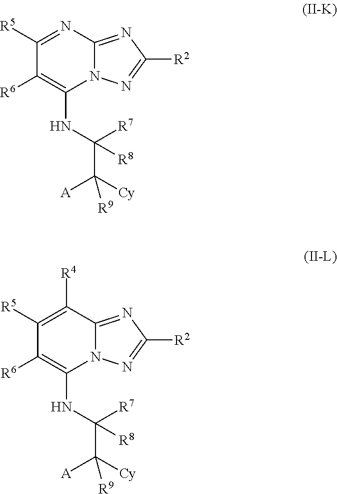

or a pharmaceutically acceptable salt thereof, wherein R¹, R², R³, R⁴, R⁵, R⁶, R⁷, R⁸, R⁹, Cy, and A are as defined herein.

In some embodiments, the compound is other than a compound of Formula (IT-A); the compound is other than a compound of Formula (II-B); the compound is other than a compound of Formula (TI-C); the compound is other than a compound of Formula (II-D); the compound is other than a compound of Formula (II-E); the compound is other than a compound of Formula (TI-F); the compound is other than a compound of Formula (II-G); the compound is other than a compound of Formula (II-H); the compound is other than a compound of Formula (TI-I); the compound is other than a compound of Formula (II-J); the compound is other than a compound of Formula (TI-K); and/or the compound is other than a compound of Formula (II-L); wherein Formulae (TI-A), (TI-B), (TI-C), (II-D), (II-E), (TI-F), (II-G), (TI-H), (TI-I), (II-J), (TI-K), and (II-L) are as defined herein.

In some embodiments, at least one of R¹, R², R³, R⁴, R⁵, and R⁶ is C₁₋₆ haloalkyl.

In some embodiments, X¹ is CR¹. In some embodiments, X¹ is N.

In some embodiments, R¹ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}NR^{d1}, NR^{c1}R^{d1}, NR^{c1}C(O)R^{b1}, NR^{c1}C(O)OR^{a1}, NR^{b1}CC(O)NR^{c1}R^{d1}, C(═NR^{c1})R^{b1}, C(═NOR^{a1})R^{b1}, C(═NR^{c1})NR^{c1}R^{d1}, NR^{c1}C(═NR^{c1})NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

In some embodiments, R¹ is selected from H, D, C₁₋₆ alkyl, C₁₋₆ haloalkyl, halo, CN, and OR^{a1}; wherein the C₁₋₆ alkyl forming R¹ is optionally substituted with 1, 2, 3, or 4 substituents each independently selected from R^{10B}. In some embodiments, R¹ is selected from H, D, C₁₋₆ alkyl (such as methyl or ethyl), C₁₋₆ haloalkyl (such as CF₃, CHF₂, CF₂CF₃), CN, and halo (such as F, Cl, or Br). In some embodiments, R¹ is H.

In some embodiments, X² is CR². In some embodiments, X² is N.

In some embodiments, R² is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}NR^{d1}, NR^{c1}R^{d1}, NR^{c1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1} NR^{a1}C(═NR^{c1})NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

In some embodiments, R² is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R² is C₁₋₆ alkyl, C₁₋₆ haloalkyl, or halo. In some embodiments, R² is C₁₋₆ haloalkyl. In some embodiments, R² is CF₃. In some embodiments, R² is H.

In some embodiments, X³ is CR³. In some embodiments, X³ is N.

In some embodiments, R³ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{a1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1}, NR^{c1}C(═NR^{a1})NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R³ are each optionally substituted with 1, 2, 3, or 4 substituents each independently selected from R^{10B}.

In some embodiments, R³ is selected from H, D, C₁₋₆ alkyl, C₁₋₆ haloalkyl, halo, CN, and OR^{a1}; wherein the C₁₋₆ alkyl forming R³ is optionally substituted with 1, 2, 3, or 4 substituents each independently selected from R^{10B}. In some embodiments, R³ is H, D, C₁₋₆ alkyl (such as methyl or ethyl), C₁₋₆ haloalkyl (such as CF₃, CHF₂, CF₂CF₃), CN, or halo (such as F, Cl, or Br).

In some embodiments, R³ is H.

In some embodiments, X⁴ is CR⁴. In some embodiments, X⁴ is N.

In some embodiments, R⁴ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}NR^{d1}, NR^{c1}R^{d1}, NR^{c1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1}, NR^{c1}C(═NR^{c1})NR^{c1}R^{d1}, NR^{a1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R⁴ are each optionally substituted with 1, 2, 3, or 4 substituents each independently selected from R^{10B}.

In some embodiments, R⁴ is selected from H, D, C₁₋₆ alkyl, C₁₋₆ haloalkyl, halo, CN, and OR^{a1}; wherein the C₁₋₆ alkyl forming R⁴ is optionally substituted with 1, 2, 3, or 4 substituents each independently selected from R^{10B}. In some embodiments, R⁴ is H, D, C₁₋₆ alkyl (such as methyl or ethyl), C₁₋₆ haloalkyl (such as CF₃, CHF₂, CF₂CF₃), CN, or halo (such as F, Cl, or Br).

In some embodiments, R⁴ is H.

In some embodiments, X⁵ is CR⁵. In some embodiments, X⁵ is N.

In some embodiments, R⁵ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{a1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1} NR^{a1}C(═NR^{c1})NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R⁵ are each optionally substituted with 1, 2, 3, or 4 substituents each independently selected from R^{10B}.

In some embodiments, R⁵ is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R⁵ is C₁₋₆ haloalkyl. In some embodiments, R⁵ is CF₃ or CHF₂. In some embodiments, R⁵ is H.

In some embodiments, X⁶ is CR⁶. In some embodiments, X⁶ is N.

In some embodiments, R⁶ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{c1})NR^{c1}R^{d1}, NR^{c1}C(═NR^{c1})NR^{c1}R^{d1}, NR^{c1}S(O)R^{b1}, NR^{c1}S(O)₂R^{b1} NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R⁶ are each optionally substituted with 1, 2, 3, or 4 substituents each independently selected from R^{10B}.

In some embodiments, R⁶ is selected from H, D, C₁₋₆ alkyl, C₁₋₆ haloalkyl, halo, CN, and OR^{a1}; wherein the C₁₋₆ alkyl forming R⁶ is optionally substituted with 1, 2, 3, or 4 substituents each independently selected from R^{10B}. In some embodiments, R⁶ is H, D, C₁₋₆ alkyl (such as methyl or ethyl), C₁₋₆ haloalkyl (such as CF₃, CHF₂, CF₂CF₃), CN, or halo (such as F, Cl, or Br).

In some embodiments, R⁶ is H.

In some embodiments, R⁷ and R⁸ are each independently selected from H, D, C₁₋₆ alkyl, C₁₋₆ haloalkyl, and halo; wherein the C₁₋₆ alkyl forming R⁷ and R⁸ are each optionally substituted with 1, 2, 3, or 4 substituents each independently selected from R^{20B}. In some embodiments, R⁷ and R⁸ are each independently selected from H, D, C₁₋₆ alkyl (such as methyl or ethyl), C₁₋₆ haloalkyl (such as CF₃, CHF₂, CF₂CF₃), and halo (such as F, Cl, or Br). In some embodiments, R⁷ and R⁸ are each H.

In some embodiments, R⁹ is selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2} SR^{a2}, C(O)R^{b2}, C(O)NR^{c2}R^{d2}, C(O)OR^{a2}, OC(O)R^{b2}, OC(O)NR^{c2}R^{d2} NR^{c2}R^{d2} NR^{c2}C(O)R^{b2}, NR^{c2}C(O)OR^{a2} NR^{c2}C(O)NR^{c2}R^{d2}, C(═NR^{c2})R^{b2}, C(═NOR^{a2})R^{b2}, C(═NR^{c2})NR^{c2}R^{d2} NR²C(═NR^{c2})NR^{c2}R^{d2} NR^{c2}S(O)R^{b2} NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{e2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁹ are each optionally substituted with 1, 2, 3, or 4 substituents independently selected from R^{30A}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{30B}.

In some embodiments, R⁹ is selected from H, D, C₁₋₆ alkyl, C₁₋₆ haloalkyl, halo, CN, and OR^{a2}; wherein the C₁₋₆ alkyl forming R⁹ is optionally substituted with 1, 2, 3, or 4 substituents each independently selected from R^{30B}. In some embodiments, R⁹ is H, D, C₁₋₆ alkyl (such as methyl or ethyl), C₁₋₆ haloalkyl (such as CF₃, CHF₂, CF₂CF₃), CN, or halo (such as F, Cl, or Br). In some embodiments, R⁹ is C₁₋₆ alkyl. In some embodiments, R⁹ is methyl or ethyl.

In some embodiments, R⁹ is selected from H and C₁₋₆ alkyl, wherein the C₁₋₆ alkyl forming R⁹ is optionally substituted with OR^{a10}. In some embodiments, R⁹ is selected from H and C₁₋₆ alkyl, wherein the C₁₋₆ alkyl forming R⁹ is optionally substituted with 1, 2, 3, or 4 substituents each independently selected from R^{30B}. In some embodiments, R⁹ is C₁₋₆ alkyl, wherein the C₁₋₆ alkyl forming R⁹ is optionally substituted with OR^{a10}. In some embodiments, R⁹ is C₁₋₆ alkyl, wherein the C₁₋₆ alkyl forming R⁹ is optionally substituted with 1, 2, 3, or 4 substituents each independently selected from R^{30B}.

In some embodiments, R⁹ is selected from H, methyl, ethyl, and CH₂OH. In some embodiments, R⁹ is selected from methyl, ethyl, and CH₂OH.

In some embodiments, A is optionally substituted with 1, 2, 3, 4, 5 or 6 substituents independently selected from R^{A}. In some embodiments, A is optionally substituted with 1, 2, 3, 4, or 5 substituents independently selected from R^{A}. In some embodiments, A is optionally substituted with 1, 2, 3, or 4 substituents independently selected from R^{A}. In some embodiments, A is optionally substituted with 1, 2, or 3 substituents independently selected from R^{A}. In some embodiments, A is optionally substituted with 1 or 2 substituents independently selected from R^{A}. In some embodiments, A is optionally substituted with 1 substituent selected from R^{A}. In some embodiments, A is unsubstituted.

In some embodiments, A is 5-10 membered heterocycloalkyl optionally substituted with 1, 2, 3, 4, 5 or 6 substituents independently selected from R^{A}. In some embodiments, A is 5-10 membered heterocycloalkyl optionally substituted with 1, 2, 3, 4, or 5 substituents independently selected from R^{A}. In some embodiments, A is 5-10 membered heterocycloalkyl optionally substituted with 1, 2, 3, or 4 substituents independently selected from R^{A}. In some embodiments, A is 5-10 membered heterocycloalkyl optionally substituted with 1, 2, or 3 substituents independently selected from R^{A}. In some embodiments, A is 5-10 membered heterocycloalkyl optionally substituted with 1 or 2 substituents independently selected from R^{A}. In some embodiments, A is 5-10 membered heterocycloalkyl optionally substituted with 1 substituent selected from R^{A}. In some embodiments, A is unsubstituted 5-10 membered heterocycloalkyl.

In some embodiments, A is selected from azetidinyl, piperidinyl, and pyrrolidinyl, each of which is unsubstituted or substituted with 1, 2, or 3 substituents independently selected from R^{A}.

In some embodiments, A is selected from azetidinyl, piperidinyl, pyrrolidinyl, 2-azabicyclo[2.2.1]heptanyl, 2-azaspiro[3.5]nonan-2-yl, 6-oxo-7-oxa-2,5-diazaspiro[3.5]nonanyl, 2,6-diazaspiro[3.3]heptanyl, 6-oxo-2,5-diazaspiro[3.4]octanyl, 7-oxo-2,6-diazaspiro[3.4]octanyl, 2-azaspiro[3.3]heptanyl, 6-oxo-2,5,7-triazaspiro[3.4]octanyl, and 6-oxo-7-oxa-2,5-diazaspiro[3.4]octan-2-yl, each of which is unsubstituted or substituted with 1, 2, or 3 substituents independently selected from R^{A}.

In some embodiments, each R^{A} is independently selected from 5-10 membered heteroaryl, C₁₋₆ alkyl, C₂₋₆ alkenyl, C₂₋₆ alkynyl, C₁₋₆ haloalkyl, halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3} NR^{c3}R^{d3} NR^{c3}C(O)R^{b3}, NR^{c3}C(O)OR^{a3}, NR^{c3}C(O)NR^{c3}R^{d3}, C(═NR^{c3})R^{b3}, C(═NOR^{a3})R^{b3}, C(═NR^{c3})NR^{c3}R^{d3} NR^{c3}C(═NR^{c3})NR^{c3}R^{d3} NR^{c3}S(O)R^{b3}, NR^{c3}S(O)₂R¹³, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein the 5-10 membered heteroaryl is optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2}.

In some embodiments, each R^{A} is selected from C₁₋₆ alkyl (such as methyl or ethyl), C₁₋₆ haloalkyl (such as CF₃, CHF₂, CF₂CF₃), halo (such as F, Cl, or Br), and OR^{a3} (such as methoxy or ethoxy). In some embodiments, each R^{A} is selected from 5-10 membered heteroaryl, C(O)NR^{c3}R^{d3}, S(O)₂NR^{c3}R^{d3}, and C(═NR^{e3})NR^{c3}R^{d3}, wherein 5-10 membered heteroaryl is optionally substituted with R^{A1}. In some embodiments, each R^{A} is selected from C(O)NH₂, S(O)₂NH₂, C(═NCN)NH₂, 5-amino-1H-1,2,4-triazol-3-yl, and 1-methyl-6-oxo-1,6-dihydropyridazin-3-yl.

In some embodiments, each R^{A} is selected from 5-10 membered heteroaryl, C₁₋₆ alkyl, OR^{a3}, NR^{c3}R^{d3}, C(O)R^{b3}, C(O)NR^{c3}R^{d3}, S(O)₂NR^{c3}R^{d3}, and C(═NR^{c3})NR^{c3}R^{d3}, wherein said 5-10 membered heteroaryl forming R^{A} is optionally substituted with R^{A1} and said C₁₋₆ alkyl forming R^{A} is optionally substituted with R^{A2}. In some embodiments, each R^{A} is selected from methyl, OH, C(O)NH₂, S(O)₂NH₂, C(═NCN)NH₂, C(O)N(H)CD₃, CH₂OH, pyridinyl, 5-amino-1H-1,2,4-triazol-3-yl, and 1-methyl-6-oxo-1,6-dihydropyridazin-3-yl.

In some embodiments, each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2}. In some embodiments, each R^{A1} is independently selected from C₁₋₆ alkyl and R^{A}.

In some embodiments, each R^{A2} is independently selected from halo, CN, OR^{a3}, SR³, C(O)R^{b3}, C(O)NR^{c3}R^{d3}, C(O)OR^{a3}, NR^{c3}R^{d3} NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}. In some embodiments, R^{A2} is NR^{c3}R^{d3} In some embodiments, each R^{A2} is independently selected from NR^{c3}R^{d3}, OR^{a3}, or C(O)NR^{c3}R^{d3}.

In some embodiments, Cy is optionally substituted with 1, 2, 3, 4, 5 or 6 substituents independently selected from R^{Cy}. In some embodiments, Cy is optionally substituted with 1, 2, 3, 4, or 5 substituents independently selected from R^{Cy}. In some embodiments, Cy is optionally substituted with 1, 2, 3, or 4 substituents independently selected from R^{Cy}. In some embodiments, Cy is optionally substituted with 1, 2, or 3 substituents independently selected from R^{Cy}. In some embodiments, Cy is optionally substituted with 1 or 2 substituents independently selected from R^{Cy}. In some embodiments, Cy is optionally substituted with 1 substituent selected from R^{Cy}. In some embodiments, Cy is unsubstituted.

In some embodiments, Cy is 5-10 membered heteroaryl optionally substituted with 1, 2, 3, 4, 5 or 6 substituents independently selected from R^{Cy}. In some embodiments, Cy is 5-10 membered heteroaryl optionally substituted with 1, 2, 3, 4, or 5 substituents independently selected from R^{Cy}. In some embodiments, Cy is 5-10 membered heteroaryl optionally substituted with 1, 2, 3, or 4 substituents independently selected from R^{Cy}. In some embodiments, Cy is 5-10 membered heteroaryl optionally substituted with 1, 2, or 3 substituents independently selected from R^{Cy}. In some embodiments, Cy is 5-10 membered heteroaryl optionally substituted with 1 or 2 substituents independently selected from R^{Cy}. In some embodiments, Cy is 5-10 membered heteroaryl optionally substituted with 1 substituent selected from R^{Cy}. In some embodiments, Cy is unsubstituted 5-10 membered heteroaryl.

In some embodiments, Cy is 5-10 membered heterocycloalkyl optionally substituted with 1, 2, 3, 4, 5 or 6 substituents independently selected from R^{Cy}. In some embodiments, Cy is 5-10 membered heterocycloalkyl optionally substituted with 1, 2, 3, 4, or 5 substituents independently selected from R^{Cy}. In some embodiments, Cy is 5-10 membered heterocycloalkyl optionally substituted with 1, 2, 3, or 4 substituents independently selected from R^{Cy}. In some embodiments, Cy is 5-10 membered heterocycloalkyl optionally substituted with 1, 2, or 3 substituents independently selected from R^{Cy}. In some embodiments, Cy is 5-10 membered heterocycloalkyl optionally substituted with 1 or 2 substituents independently selected from R^{Cy}. In some embodiments, Cy is 5-10 membered heterocycloalkyl optionally substituted with 1 substituent selected from R^{Cy}. In some embodiments, Cy is unsubstituted 5-10 membered heterocycloalkyl.

In some embodiments, Cy is phenyl substituted with 1, 2, 3, 4, or 5 substituents independently selected from R^{Cy}. In some embodiments, Cy is phenyl substituted with 1, 2, 3, or 4 substituents independently selected from R^{Cy}. In some embodiments, Cy is phenyl substituted with 1, 2, or 3 substituents independently selected from R^{Cy}. In some embodiments, Cy is phenyl substituted with 1 or 2 substituents independently selected from R^{Cy}. In some embodiments, Cy is phenyl substituted with 1 substituent selected from R^{Cy}. In some embodiments, Cy is unsubstituted phenyl. In some embodiments, Cy is selected from 4-halophenyl and phenyl. In some embodiments, Cy is selected from 4-fluorophenyl and phenyl.

In some embodiments, each R^{Cy} is independently selected from C₁₋₆ alkyl, C₂₋₆ alkenyl, C₂₋₆ alkynyl, C₁₋₆ haloalkyl, halo, CN, NO₂, OR^{a5}, SR^{a5}, C(O)R^{b5}, C(O)NR^{c5}SR^{d5}, C(O)OR^{a5} OC(O)R^{b5}, OC(O)NR^{c5}R^{d5} NR^{c5}R^{d5} NR^{c5}C(O)R^{b5}, NR^{c5}C(O)OR^{a5}, NR^{c5}C(O)NR^{c5}R^{d5}, C(═NR^{c5})R^{d5}, C(═NOR^{a5})R^{b5}, C(═NR^{c5})NR^{c5}R^{d5} NR^{c5}C(═NR^{c5})NR^{c5}R^{d5} NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2} In some embodiments, each R^{Cy} is selected from C₁₋₆ alkyl (such as methyl or ethyl), C₁₋₆ haloalkyl (such as CF₃, CHF₂, CF₂CF₃), halo (such as F, Cl, or Br), and OR^{a2} (such as methoxy or ethoxy). In some embodiments, each R^{Cy} is selected from halo. In some embodiments, each R^{Cy} is F.

Also provided herein is a compound having Formula (II-A):

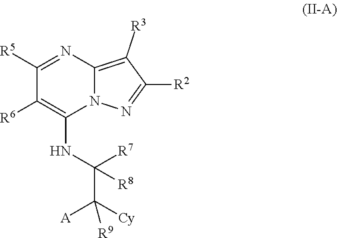

or a pharmaceutically acceptable salt thereof, wherein:
R², R³, R⁵, and R⁶ are each independently selected from H, D, C₁₋₆ alkyl, C₂₋₆ alkenyl, C₂-6 alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NOR^{a1})R^{b1}, C(═NR^{e1})NR^{c1}R^{d1} NR^{c1}C(═NR^{c1})NR^{c1}R^{d1} NR^{a1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R², R³, R⁵, and R⁶ are each optionally substituted with 1, 2, 3, or 4 substituents each independently selected from R^{10A}; and wherein the C₁₋₆ alkyl, C₂₋₆ alkenyl, and C₂₋₆ alkynyl forming R², R³, R⁵, and R⁶ are each optionally substituted with 1, 2, 3, or 4 substituents each independently selected from R^{10B};
R⁷ and R⁸ are each independently selected from H, D, C₁₋₆ alkyl, C₂₋₆ alkenyl, C₂₋₆ alkynyl, C₁₋₆ haloalkyl, halo, C₃₋₁₀ cycloalkyl, and 4-10 membered heterocycloalkyl, wherein the C₃₋₁₀ cycloalkyl and 4-10 membered heterocycloalkyl forming R⁷ and R⁸ are each optionally substituted with 1, 2, 3, or 4 substituents independently selected from R^{20A}; and wherein the C₁₋₆ alkyl, C₂₋₆ alkenyl, and C₂₋₆ alkynyl forming R⁷ and R⁸ are each optionally substituted with 1, 2, 3, or 4 substituents independently selected from R^{20B};
R⁹ is selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2} SR^{a2}, C(O)R^{b2}, C(O)NR^{c2}R^{d2}, C(O)OR^{a2}, OC(O)R^{b2}, OC(O)NR^{c2}R^{d2} NR^{c2}R^{d2} NR^{c2}C(O)R^{b2} NR^{c2}C(O)OR^{a2} NR^{c2}C(O)NR^{c2}R^{d2}, C(═NR^{c2})R^{b2}, C(═NOR^{a2})R^{b2}, C(═NR^{c2})NR^{c2}R^{d2} NR^{c2}C(═NR^{c2})R^{c2}R^{d2} NR^{c2}S(O)R^{b2} NR^{c2}S(O)₂R^{b2} NR^{c2}S(O)₂NR^{c2}R^{d2} S(O)R^{b2}, S(O)NR^{c2}R^{d2}, S(O)₂R^{b2}, and S(O)₂NR^{c2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁹ are each optionally substituted with 1, 2, 3, or 4 substituents independently selected from R^{30A}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{30B};
A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A}; Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR³, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{c3})R^{b3}, C(═NOR^{a3})R^{b3}, C(═NR^{c3})NR^{c3}R^{d3}, NR^{c3}C(═NR^{c3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR³, SR^{a3}, C(O)R^{b3}, C(O)NR^{c3}R^{d3}, C(O)OR^{a}, NR^{c3}R^{d3} NR^{c3}C(O)R^{b3}NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4} NR^{c4}R^{d4} NR^{c4}C(O)R^{b4}, NR^{c4}C(O)OR^{a4}, NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR^{c4})NR^{c4}R^{d4} NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R⁹¹; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR⁵C(═NR⁵)NR^{c5}R^{d5} NR^{c5}S(O)R^{b5} NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{c6}C(O)R^{b6}NR^{c6}C(O)OR^{a6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6} S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R⁹¹;
each R^{10A} is independently selected from C₁₋₆ alkyl, C₂₋₆ alkenyl, C₂₋₆ alkynyl, C₁₋₆ haloalkyl, and R^{10B}; wherein the C₁₋₆ alkyl, C₂₋₆ alkenyl, and C₂₋₆ alkynyl forming R^{10A} are each optionally substituted with 1, 2, 3, or 4 substituents independently selected from R^{10B};
each R^{10B} is independently selected from C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7} NR^{c7}R^{d7} NR^{c7}C(O)R^{b7} NR^{c7}C(O)OR^{a7} NR^{c7}C(O)NR^{c7}R^{d7}C(═NR^{c7})R^{b7}, C(═NOR^{a7})R^{b7}, C(═NR^{c7})NR^{c7}R^{d7} NR^{c7}C(═NR^{c7})NR^{c7}R^{d7} NR^{c7}S(O)R^{b7} NR^{c7}S(O)₂R^{b7} NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{10B} are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a8}, SR^{a8}, C(O)R^{b8}, C(O)NR^{c8}R^{d8}, C(O)OR^{a8}, NR^{c8}R^{d8} NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹¹ are each optionally substituted with 1, 2, 3, or 4 substituents independently selected from R^{g1}; and wherein the C₁₋₆ alkyl, C₂₋₆ alkenyl, and C₂₋₆ alkynyl forming R¹¹ are each optionally substituted with 1, 2, 3, or 4 substituents independently selected from R^{g2};
each R^{20A} is independently selected from C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{20A} are each optionally substituted with 1, 2, 3, or 4 substituents independently selected from R^{20B};
each R^{20B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a9}, SR^{a9}, C(O)R^{b9}, C(O)NR^{c9}R^{d9}, C(O)OR^{a9}, NR^{c9}R^{d9} NR^{c9}C(O)R^{b9}, NR^{c9}C(O)OR^{a9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9} S(O)₂R^{b9}, and S(O)₂NR^{c9}R^{d9}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{20B} are each optionally substituted with 1, 2, 3, or 4 substituents independently selected from R^{g1};
each R^{30A} is independently selected from C₁₋₆ alkyl, C₂₋₆ alkenyl, C₂₋₆ alkynyl, C₁₋₆ haloalkyl, and R^{30B}; wherein the C₁₋₆ alkyl, C₂₋₆ alkenyl, and C₂₋₆ alkynyl forming R^{30A} are each optionally substituted with 1, 2, 3, or 4 substituents independently selected from R^{30B};
each R^{30B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a10}, SR^{a10}, C(O)R^{b10}, C(O)NR^{c10}R^{d10}, C(O)OR^{a10}, NR^{c10}R^{d10} NR^{c10}OC(O)R^{b10}, NR^{c10}C(O)OR^{a10}, NR^{c10}S(O)R^{b10}, NR^{c10}S(O)₂R^{b10} NR^{c10}S(O)₂NR^{c10}R^{d10} S(O)R^{b10}, S(O)NR^{c10}R^{d10}, S(O)₂R^{b10}, and S(O)₂NR^{c10}R^{d10}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{30B} are each optionally substituted with 1, 2, 3, or 4 substituents independently selected from R^{g1};
each R^{a1}, R^{c1}, R^{d1}, R^{a2}, R^{c2}, R^{d2}, R^{a3}, R^{c3}, R^{d3}, R^{a4}, R^{c4}, R^{d4}, R^{a8}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a8}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10} and R^{d10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a8}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} are each optionally substituted with 1, 2, 3, or 4 substituents independently selected from R^{g2};
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c8} and R^{d8} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c9} and R^{d9} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, R^{b3}, R^{b4}, R^{b5}, R^{b6}, R^{b7}, R^{b8}, R^{b9}, and R^{b10} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b1}, R^{b2}, R^{b3}, R^{b4}, R^{b5}, R^{b6}, R^{b7}, R^{b8}, R^{b9}, and R^{b10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b1}, R^{b2}, R^{b3}, R^{b4}, R^{b5}, R^{b6}, R^{b7}, R^{b8}, R^{b9}, and R^{b10} are each optionally substituted with 1, 2, 3, or 4 substituents independently selected from R^{g2};
each R^{c1}, R^{c2}, R^{c3}, R^{c4}, R^{c5} and R^{c7} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2} wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}R^{d11}NR^{c11}C(O)R^{b11}, NR^{c11}C(O)OR^{a11}, NR^{b11}S(O)R^{b11}, NR^{c11}S(O)₂R^{b1}, NR^{c11}S(O)₂NR^{c11}R^{d11}, S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c11}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a1}, R^{c}, and R^{d11} are each optionally substituted with 1, 2, 3, or 4 substituents independently selected from R^{h2};
each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2};
each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Also provided herein is a compound having Formula (II-A):

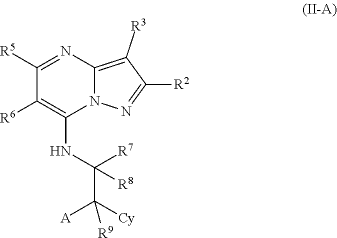

or a pharmaceutically acceptable salt thereof, wherein:
   R², R³, R⁵, and R⁶ are each independently selected from H, D, C₁₋₆ alkyl, C₁₋₆ haloalkyl, CN, or halo;
   R⁷ and R⁸ are each independently selected from H, D, C₁₋₆ alkyl, C₁₋₆ haloalkyl, CN, or halo;
   R⁹ is selected from H, D, C₁₋₆ alkyl, and C₁₋₆ haloalkyl, wherein the C₁₋₆ alkyl forming R⁹ is optionally substituted with OR^{a10};
   A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A}; Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
   each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a}, SR^{a3}, C(O)R^{b3}, C(O)NR^{c3}R^{d3}, C(O)OR^{a3}, OC(O)R^{b3}, OC(O)NR^{c3}R^{d3} NR^{c3}R^{d3} NR^{c3}C(O)R^{b3}, NR^{c3}C(O)OR^{a3} NR^{c3}C(O)NR^{c3}R^{d3}C(═NR^{c3})R^{b3}, C(═NOR^{a3})R^{b3}, C(═NR^{c3})NR^{c3}R^{d3} NR^{c3}C(═NR^{c3})NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
   each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
   each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR³, SR^{a3}, C(O)R^{b3}, C(O)NR^{c3}R^{d3}, C(O)OR^{a}, NR^{c3}R^{d3} NR^{c3}C(O)R^{b3}NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
   each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4}, NR^{c4}C(O)OR^{a4} NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR⁴)NR^{c4}R^{d4} NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R^{g1}; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
   each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR⁵C(═NR^{e5})NR^{c5}R^{d5} NR^{c5}S(O)R^{d5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
   each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
   each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{c6}C(O)R^{b6}NR^{c6}C(O)OR^{a6}, NR^{c6}S(O)R^{b6}, NR^{c6}S(O)₂R^{b6}, NR^{c6}S(O)₂NR^{c6}R^{d6}, S(O)R^{b6}, S(O)NR⁶R^{d6} S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R^{g1};
   each R^{a3}, R^{c3}, R^{d3}, R^{a4}, R^{c4}, R^{d4}, R^{a5}, R^{c5}, R^{d5}, R^{a6}, R^{c6}, R^{d6} and R^{a10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{c3}, R^{d3}, R^{a4}, R^{c4}, R^{d4}, R^{a5}, R^{c5}, R^{d5}, R^{a6}, R^{c6}, R^{d6}, and R^{a10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a3}, R^{c3}, R^{d3}, R^{a4}, R^{c4}, R^{d4}, R^{a8}, R^{c5}, R^{d5}, R^{a6}, R^{c6}, R^{d6}, and R^{a10} are each optionally substituted with 1, 2, 3, or 4 substituents independently selected from R^{g2}; or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
   each R^{b3}, R^{b4}, R^{b5}, and R^{b6} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b3}, R^{b4}, R^{b5}, and R^{b6} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b3}, R^{b4}, R^{b5}, and R^{b6} are each optionally substituted with 1, 2, 3, or 4 substituents independently selected from R^{g2};
   each R^{c3}, R^{c4}, and R^{c5} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
   each R^{g1} is independently selected from C₁₋₆ alkyl, C₂₋₆ alkenyl, C₂₋₆ alkynyl, and R^{g2} wherein the C₁₋₆ alkyl, C₂₋₆ alkenyl, and C₂₋₆ alkynyl are each optionally substituted with 1, 2, or 3 substituents independently selected from R^{g2};
   each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}R^{d11} NR^{c11}C(O)R^{b11}, NR^{c11}C(O)OR^{a11}, NR^{c11}S(O)R^{b11}, NR^{c11}S(O)₂R^{b11}, NR^{c11}S(O)₂NR^{c11}R^{d11} S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
   each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c11}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a11}, R^{c11}, and R^{d11} are each optionally substituted with 1, 2, 3, or 4 substituents independently selected from R^{h2};
   each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
   each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2}; and
   each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Also provided herein is a compound having Formula (II-A):

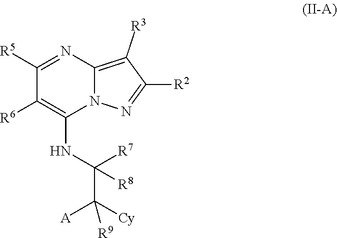

or a pharmaceutically acceptable salt thereof, wherein:
   R², R³, R⁵, and R⁶ are each independently selected from H, D, C₁₋₆ alkyl, C₁₋₆ haloalkyl, CN, or halo;
   R⁷ and R⁸ are each independently selected from H, D, C₁₋₆ alkyl, C₁₋₆ haloalkyl, CN, or halo;
   R⁹ is selected from H, D, C₁₋₆ alkyl, and C₁₋₆ haloalkyl;
   A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A}; Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
   each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3} NR^{c3}R^{d3} NR^{c3}C(O)R^{b3}, NR^{c3}C(O)OR^{a3} NR^{c3}C(O)NR^{c3}R^{d3}C(═NR^{c3})R^{b3}, C(═NOR^{a3})R^{b3}, C(═NR^{c3})NR^{c3}R^{d3} NR^{c3}C(═NR^{c3})NR³R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
   each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
   each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR³, SR^{a3}, C(O)R^{b3}, C(O)NR^{c3}R^{d3}, C(O)OR^{a}, NR^{c3}R^{d3} NR^{c3}C(O)R^{b3}NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
   each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4} NR^{c4}R^{d4} NR^{c4}C(O)R^{b4}, NR^{c4}C(O)OR^{a4} NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR^{c4})NR^{c4}R^{d4} NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R⁹¹; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
   each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR⁵C(═NR⁵)NR^{c5}R^{d5} NR^{c5}S(O)R^{b5} NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
   each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
   each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{c6}C(O)R^{b6}NR^{c6}C(O)OR^{a6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6} S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R^{g1};
   each R^{a3}, R^{c3}, R^{d3}, R^{a4}, R^{c4}, R^{d4}, R^{a5}, R^{c5}, R^{d5}, R^{a6}, R^{c6}, and R^{d6} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{c3}, R^{d3}, R^{a4}, R^{c4}, R^{d4}, R^{a5}, R^{c5}, R^{d5}, R^{a6}, R^{c6}, and R^{d6} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a3}, R^{c3}, R^{d3}, R^{a4}, R^{c4}, R^{d4}, R^{a5}, R^{c5}, R^{d5}, R^{a6}, R^{c6}, and R^{d6} are each optionally substituted with 1, 2, 3, or 4 substituents independently selected from R^{g2};
   or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
   or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
   each R^{b3}, R^{b4}, R^{b5}, and R^{b6} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b3}, R^{b4}, R^{b5}, and R^{b6} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b3}, R^{b4}, R^{b5}, and R^{b6} are each optionally substituted with 1, 2, 3, or 4 substituents independently selected from R^{g2};
   each R^{c3}, R^{c4}, and R^{c5} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
   each R^{g1} is independently selected from C₁₋₆ alkyl, C₂₋₆ alkenyl, C₂₋₆ alkynyl, and R^{g2} wherein the C₁₋₆ alkyl, C₂₋₆ alkenyl, and C₂₋₆ alkynyl are each optionally substituted with 1, 2, or 3 substituents independently selected from R^{g2};
   each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}R^{d11} NR^{c11}C(O)R^{d11}, NR^{c11}C(O)OR^{a11}, NR^{c11}S(O)R^{b11}, NR^{c11}S(O)₂R^{b1}, NR^{c11}S(O)₂NR^{c11}R^{d11} S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
   each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c11}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a11}, R^{c11}, and R^{d11} are each optionally substituted with 1, 2, 3, or 4 substituents independently selected from R^{h2};
   each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
   each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2}; and
   each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Also provided herein is a compound having Formula (II-A):

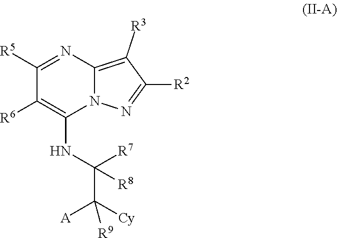

or a pharmaceutically acceptable salt thereof, wherein:
   R², R³, R⁵, and R⁶ are each independently selected from H, D, and C₁₋₆ haloalkyl;
   R⁷ and R⁸ are each independently selected from H and D;
   R⁹ is selected from H and C₁₋₆ alkyl, wherein said C₁₋₆ alkyl is optionally substituted with OR^{a10};
   A is a ring selected from 4-12 membered heterocycloalkyl, wherein the 4-12 membered heterocycloalkyl forming A is optionally substituted with 1, 2, 3, 4, 5, or 6 substituents independently selected from R^{A};
   Cy is phenyl optionally substituted with 1, 2, 3, 4, or 5 substituents independently selected from R^{Cy};
   each R^{A} is selected from 5-10 membered heteroaryl, C₁₋₆ alkyl, OR^{a3}, NR^{c3}R^{d3}, C(O)R^{b3}, C(O)NR^{c3}R^{d3}, S(O)₂NR^{c3}R^{d3}, and C(═NR^{e3})NR^{c3}R^{d3}, wherein said 5-10 membered heteroaryl is optionally substituted with R^{A1} and said C₁₋₆ alkyl is optionally substituted with R^{A2};
   each R^{A}1 is independently selected from C₁₋₆ alkyl and R^{A2};
   each R^{A2} is NR^{c3}R^{d3}, C(O)NR^{c3}R^{d3}, and OR^{a3};
   each R^{Cy} is independently selected from halo;
   each R^{a3}, R^{c3}, R^{d3}, and R^{a10} is independently selected from H and C₁₋₆ alkyl; and
   each R^{c3} is independently selected from H, CN, and C₁₋₆ alkyl.

Also provided herein is a compound having Formula (II-A):

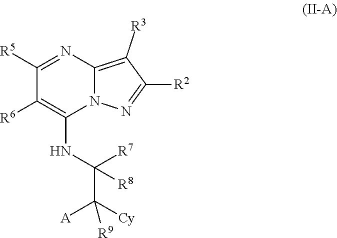

or a pharmaceutically acceptable salt thereof, wherein:
   R², R³, R⁵, and R⁶ are each independently selected from H, D, and C₁₋₆ haloalkyl;
   R⁷ and R⁸ are each independently selected from H and D;
   R⁹ is selected from C₁₋₆ alkyl;
   A is a ring selected from 4-12 membered heterocycloalkyl, wherein the 4-12 membered heterocycloalkyl forming A is optionally substituted with 1, 2, 3, 4, 5, or 6 substituents independently selected from R^{A}; Cy is phenyl optionally substituted with 1, 2, 3, 4, or 5 substituents independently selected from R^{Cy};
   each R^{A} is selected from 5-10 membered heteroaryl, C(O)NR^{c3}R^{d3}, S(O)₂NR^{c3}R^{d3}, and C(═NR^{e3})NR^{c3}R^{d3}, wherein the 5-10 membered heteroaryl is optionally substituted with R^{A1};
   each R^{A}1 is independently selected from C₁₋₆ alkyl and R^{A2};
   each R^{A2} is NR^{c3}R^{d3};
   each R^{Cy} is independently selected from halo;
   each R^{c3} and R^{d3} is independently selected from H and C₁₋₆ alkyl; and
   each R^{c3} is independently selected from H, CN, and C₁₋₆ alkyl.

In some embodiments, the compound of Formula (I) is selected from:
3-(1-((2,5-bis(trifluoromethyl)pyrazolo[1,5-a]pyrimidin-7-yl)amino)-2-(4-fluorophenyl)butan-2-yl)azetidine-1-carboxamide;
4-(1-((2,5-bis(trifluoromethyl)pyrazolo[1,5-a]pyrimidin-7-yl)amino)-2-(4-fluorophenyl)propan-2-yl)piperidine-1-carboxamide;
3-(1-((2,5-bis(trifluoromethyl)pyrazolo[1,5-a]pyrimidin-7-yl)amino)-2-(4-fluorophenyl)propan-2-yl)pyrrolidine-1-carboxamide;
3-(1-((2,5-bis(trifluoromethyl)pyrazolo[1,5-a]pyrimidin-7-yl)amino)-2-(4-fluorophenyl)propan-2-yl)azetidine-1-carboxamide;
3-(1-((5-(difluoromethyl)-2-(trifluoromethyl)pyrazolo[1,5-a]pyrimidin-7-yl)amino)-2-phenylpropan-2-yl)azetidine-1-sulfonamide;
3-(1-((5-(difluoromethyl)-2-(trifluoromethyl)pyrazolo[1,5-a]pyrimidin-7-yl)amino)-2-phenylpropan-2-yl)azetidine-1-carboxamide;
N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine;
3-(1-((2,5-bis(trifluoromethyl)pyrazolo[1,5-a]pyrimidin-7-yl)amino)-2-phenylpropan-2-yl)azetidine-1-carboxamide; N-(2-(azetidin-3-yl)-2-phenylpropyl)-2,5-bis(trifluoromethyl)pyrazolo[1,5-a]pyrimidin-7-amine; 6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one; N-(2-(azetidin-3-yl)-2-(4-fluorophenyl)propyl)-2,5-bis(trifluoromethyl)pyrazolo[1,5-a]pyrimidin-7-amine; and 3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide, or a pharmaceutically acceptable salt of any of the aforementioned.

In some embodiments, the compound of Formula (I) is selected from:
4-(2-((2,5-bis(trifluoromethyl)pyrazolo[1,5-a]pyrimidin-7-yl)amino)-1-(4-fluorophenyl)ethyl)piperidine-1-carboxamide;
3-(1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
2-(3-(1-((2,5-bis(trifluoromethyl)pyrazolo[1,5-a]pyrimidin-7-yl)amino)-2-(4-fluorophenyl)propan-2-yl)pyrrolidin-1-yl)acetamide;
3-(1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
3-(2-((2,5-bis(trifluoromethyl)pyrazolo[1,5-a]pyrimidin-7-yl)amino)-1-(4-fluorophenyl)ethyl)pyrrolidine-1-carboxamide;
3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide;
2-(2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol;
N-(2-(4-fluorophenyl)-2-(3-(pyridin-2-yl)azetidin-1-yl)ethyl)-2,5-bis(trifluoromethyl)pyrazolo[1,5-a]pyrimidin-7-amine;
2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one;
1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide;
N-(2-(4-fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine;
N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine;
6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide;
(3R)-1-(2-((2,5-bis(trifluoromethyl)pyrazolo[1,5-a]pyrimidin-7-yl)amino)-1-(4-fluorophenyl)ethyl)-3-methylpyrrolidin-3-ol;
((3S)-1-(2-((2,5-bis(trifluoromethyl)pyrazolo[1,5-a]pyrimidin-7-yl)amino)-1-(4-fluorophenyl)ethyl)pyrrolidin-3-yl)methanol;
2-(2-((2,5-bis(trifluoromethyl)pyrazolo[1,5-a]pyrimidin-7-yl)amino)-1-(4-fluorophenyl)ethyl)-2,5-diazaspiro[3.4]octan-6-one;
6-(2-((2,5-bis(trifluoromethyl)pyrazolo[1,5-a]pyrimidin-7-yl)amino)-1-(4-fluorophenyl)ethyl)-2,6-diazaspiro[3.4]octane-2-carboxamide;
2-(2-((2,5-bis(trifluoromethyl)pyrazolo[1,5-a]pyrimidin-7-yl)amino)-1-(4-fluorophenyl)ethyl)-2-azaspiro[3.3]heptan-5-ol;
1-(2-((2,5-bis(trifluoromethyl)pyrazolo[1,5-a]pyrimidin-7-yl)amino)-1-(4-fluorophenyl)ethyl)-3-methylazetidin-3-ol;
2-(2-((2,5-bis(trifluoromethyl)pyrazolo[1,5-a]pyrimidin-7-yl)amino)-1-(4-fluorophenyl)ethyl)-2,6-diazaspiro[3.4]octan-7-one;
2-(2-((2,5-bis(trifluoromethyl)pyrazolo[1,5-a]pyrimidin-7-yl)amino)-1-(4-fluorophenyl)ethyl)-2,5,7-triazaspiro[3.4]octan-6-one;
1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile;
1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one;
6-(2-((2,5-bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide;
6-(2-((2,5-bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide;
2-(2-((2,5-bis(trifluoromethyl)pyrazolo[1,5-a]pyrimidin-7-yl)amino)-1-(4-fluorophenyl)ethyl)-7-oxa-2,5-diazaspiro[3.4]octan-6-one,
or a pharmaceutically acceptable salt of any of the aforementioned.
In some embodiments, the compound of Formula (I) is selected from:
(R)-3-(1-((2,5-bis(trifluoromethyl)pyrazolo[1,5-a]pyrimidin-7-yl)amino)-2-(4-fluorophenyl)butan-2-yl)azetidine-1-carboxamide;
(S)-3-(1-((2,5-bis(trifluoromethyl)pyrazolo[1,5-a]pyrimidin-7-yl)amino)-2-(4-fluorophenyl)butan-2-yl)azetidine-1-carboxamide;
(R)-4-(1-((2,5-bis(trifluoromethyl)pyrazolo[1,5-a]pyrimidin-7-yl)amino)-2-(4-fluorophenyl)propan-2-yl)piperidine-1-carboxamide;
(S)-4-(1-((2,5-bis(trifluoromethyl)pyrazolo[1,5-a]pyrimidin-7-yl)amino)-2-(4-fluorophenyl)propan-2-yl)piperidine-1-carboxamide;
(R*)-3-((R*)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R*)-3-((S*)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R)-3-((R)-1-((2,5-bis(trifluoromethyl)pyrazolo[1,5-a]pyrimidin-7-yl)amino)-2-(4-fluorophenyl)propan-2-yl)pyrrolidine-1-carboxamide;
(S)-3-((S)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R)-3-((S)-1-((2,5-bis(trifluoromethyl)pyrazolo[1,5-a]pyrimidin-7-yl)amino)-2-(4-fluorophenyl)propan-2-yl)pyrrolidine-1-carboxamide;
(S)-3-((R)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R)-3-(1-((2,5-bis(trifluoromethyl)pyrazolo[1,5-a]pyrimidin-7-yl)amino)-2-(4-fluorophenyl)propan-2-yl)azetidine-1-carboxamide;
(S)-3-(1-((2,5-bis(trifluoromethyl)pyrazolo[1,5-a]pyrimidin-7-yl)amino)-2-(4-fluorophenyl)propan-2-yl)azetidine-1-carboxamide;
(R)-3-(1-((5-(difluoromethyl)-2-(trifluoromethyl)pyrazolo[1,5-a]pyrimidin-7-yl)amino)-2-phenylpropan-2-yl)azetidine-1-sulfonamide;
(S)-3-(1-((5-(difluoromethyl)-2-(trifluoromethyl)pyrazolo[1,5-a]pyrimidin-7-yl)amino)-2-phenylpropan-2-yl)azetidine-1-sulfonamide;
(R)—N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine;
(S)—N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine;
(R)-3-(1-((2,5-bis(trifluoromethyl)pyrazolo[1,5-a]pyrimidin-7-yl)amino)-2-phenylpropan-2-yl)azetidine-1-carboxamide;
(S)-3-(1-((2,5-bis(trifluoromethyl)pyrazolo[1,5-a]pyrimidin-7-yl)amino)-2-phenylpropan-2-yl)azetidine-1-carboxamide;
(R)—N-(2-(azetidin-3-yl)-2-phenylpropyl)-2,5-bis(trifluoromethyl)pyrazolo[1,5-a]pyrimidin-7-amine;
(S)—N-(2-(azetidin-3-yl)-2-phenylpropyl)-2,5-bis(trifluoromethyl)pyrazolo[1,5-a]pyrimidin-7-amine;
(R)-6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one;
(S)-6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one;
(R)—N-(2-(azetidin-3-yl)-2-(4-fluorophenyl)propyl)-2,5-bis(trifluoromethyl)pyrazolo[1,5-a]pyrimidin-7-amine;
(S)—N-(2-(azetidin-3-yl)-2-(4-fluorophenyl)propyl)-2,5-bis(trifluoromethyl)pyrazolo[1,5-a]pyrimidin-7-amine;
(R)-3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide; and
(S)-3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide,
or a pharmaceutically acceptable salt of any of the aforementioned.
In some embodiments, the compound of Formula (I) is selected from:
(R)-4-(2-((2,5-bis(trifluoromethyl)pyrazolo[1,5-a]pyrimidin-7-yl)amino)-1-(4-fluorophenyl)ethyl)piperidine-1-carboxamide;
(S)-4-(2-((2,5-bis(trifluoromethyl)pyrazolo[1,5-a]pyrimidin-7-yl)amino)-1-(4-fluorophenyl)ethyl)piperidine-1-carboxamide;
(R*)-3-((R*)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(R)-3-((R)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(S)-3-((S)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
R*)-3-((S*)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(R)-3-((S)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(S)-3-((R)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
2-((R*)-3.-((R*)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((R)-3-((R)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((S)-3-((S)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((R*)-3-((S*)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((R)-3-((S)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((S)-3-((R)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
(R*)-3-((S*)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R)-3-((S)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(S)-3-((R)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R*)-3-((R*)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R)-3-((R)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(S)-3-((S)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R*)-3-((R*)-2-((2,5-bis(trifluoromethyl)pyrazolo[1,5-a]pyrimidin-7-yl)amino)-1-(4-fluorophenyl)ethyl)pyrrolidine-1-carboxamide;
(R)-3-((R)-2-((2,5-bis(trifluoromethyl)pyrazolo[1,5-a]pyrimidin-7-yl)amino)-1-(4-fluorophenyl)ethyl)pyrrolidine-1-carboxamide;
(S)-3-((S)-2-((2,5-bis(trifluoromethyl)pyrazolo[1,5-a]pyrimidin-7-yl)amino)-1-(4-fluorophenyl)ethyl)pyrrolidine-1-carboxamide;
(R*)-3-((S*)-2-((2,5-bis(trifluoromethyl)pyrazolo[1,5-a]pyrimidin-7-yl)amino)-1-(4-fluorophenyl)ethyl)pyrrolidine-1-carboxamide;
(R)-3-((S)-2-((2,5-bis(trifluoromethyl)pyrazolo[1,5-a]pyrimidin-7-yl)amino)-1-(4-fluorophenyl)ethyl)pyrrolidine-1-carboxamide;
(S)-3-((R)-2-((2,5-bis(trifluoromethyl)pyrazolo[1,5-a]pyrimidin-7-yl)amino)-1-(4-fluorophenyl)ethyl)pyrrolidine-1-carboxamide;
(R)-3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide;
(S)-3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide;
(1R*,4R*)-2-((R*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4R)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4S)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R*,4R*)-2-((S*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4R)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4S)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S*,4R*)-2-((S*)-2-((2, 5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4R)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4S)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S*,4R*)-2-((R*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4R)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4S)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(R)-2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol;
(S)-2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol;
(R)—N-(2-(4-fluorophenyl)-2-(3-(pyridin-2-yl)azetidin-1-yl)ethyl)-2,5-bis(trifluoromethyl)pyrazolo[1,5-a]pyrimidin-7-amine;
(S)—N-(2-(4-fluorophenyl)-2-(3-(pyridin-2-yl)azetidin-1-yl)ethyl)-2,5-bis(trifluoromethyl)pyrazolo[1,5-a]pyrimidin-7-amine;
(R)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one;
(S)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one;
(R)-1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide;
(S)-1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide;
(R)—N-(2-(4-fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine;
(S)—N-(2-(4-fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine;
(R)—N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine;
(S)—N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine;
(R)-6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide;
(S)-6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide;
(R)-1-((R)-2-((2,5-bis(trifluoromethyl)pyrazolo[1,5-a]pyrimidin-7-yl)amino)-1-(4-fluorophenyl)ethyl)-3-methylpyrrolidin-3-ol;
(R)-1-((S)-2-((2,5-bis(trifluoromethyl)pyrazolo[1,5-a]pyrimidin-7-yl)amino)-1-(4-fluorophenyl)ethyl)-3-methylpyrrolidin-3-ol;
((S)-1-((R)-2-((2,5-bis(trifluoromethyl)pyrazolo[1,5-a]pyrimidin-7-yl)amino)-1-(4-fluorophenyl)ethyl)pyrrolidin-3-yl)methanol;
((S)-1-((S)-2-((2,5-bis(trifluoromethyl)pyrazolo[1,5-a]pyrimidin-7-yl)amino)-1-(4-fluorophenyl)ethyl)pyrrolidin-3-yl)methanol;
(R)-2-(2-((2,5-bis(trifluoromethyl)pyrazolo[1,5-a]pyrimidin-7-yl)amino)-1-(4-fluorophenyl)ethyl)-2,5-diazaspiro[3.4]octan-6-one;
(S)-2-(2-((2,5-bis(trifluoromethyl)pyrazolo[1,5-a]pyrimidin-7-yl)amino)-1-(4-fluorophenyl)ethyl)-2,5-diazaspiro[3.4]octan-6-one;
(R)-6-(2-((2,5-bis(trifluoromethyl)pyrazolo[1,5-a]pyrimidin-7-yl)amino)-1-(4-fluorophenyl)ethyl)-2,6-diazaspiro[3.4]octane-2-carboxamide;
(S)-6-(2-((2,5-bis(trifluoromethyl)pyrazolo[1,5-a]pyrimidin-7-yl)amino)-1-(4-fluorophenyl)ethyl)-2,6-diazaspiro[3.4]octane-2-carboxamide;
(S*)-2-((R*)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(S)-2-((R)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(R)-2-((S)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(S*)-2-((S*)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(S)-2-((S)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(R)-2-((R)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(R)-1-(2-((2,5-bis(trifluoromethyl)pyrazolo[1,5-a]pyrimidin-7-yl)amino)-1-(4-fluorophenyl)ethyl)-3-methylazetidin-3-ol;
(S)-1-(2-((2,5-bis(trifluoromethyl)pyrazolo[1,5-a]pyrimidin-7-yl)amino)-1-(4-fluorophenyl)ethyl)-3-methylazetidin-3-ol;
(R)-2-(2-((2,5-bis(trifluoromethyl)pyrazolo[1,5-a]pyrimidin-7-yl)amino)-1-(4-fluorophenyl)ethyl)-2,6-diazaspiro[3.4]octan-7-one;
(S)-2-(2-((2,5-bis(trifluoromethyl)pyrazolo[1,5-a]pyrimidin-7-yl)amino)-1-(4-fluorophenyl)ethyl)-2,6-diazaspiro[3.4]octan-7-one;
(R)-2-(2-((2,5-bis(trifluoromethyl)pyrazolo[1,5-a]pyrimidin-7-yl)amino)-1-(4-fluorophenyl)ethyl)-2,5,7-triazaspiro[3.4]octan-6-one;
(S)-2-(2-((2,5-bis(trifluoromethyl)pyrazolo[1,5-a]pyrimidin-7-yl)amino)-1-(4-fluorophenyl)ethyl)-2,5,7-triazaspiro[3.4]octan-6-one;
(R)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile;
(S)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile;
(R)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one;
(S)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one;
(R)-6-(2-((2,5-bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide;
(S)-6-(2-((2,5-bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide;
(R)-6-(2-((2,5-bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide;
(S)-6-(2-((2,5-bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide;
(R)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.4]octan-6-one; and
(S)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.4]octan-6-one,
   or a pharmaceutically acceptable salt of any of the aforementioned.

In other embodiments, the compound of Formula (I) is in the form of a pharmaceutically acceptable salt. In other embodiments, the compound of Formula (I) is in the form of a free base or free acid, or other than in the form of a salt.

In another aspect, provided herein is a pharmaceutical composition comprising a compound of Formula (I), or any of the embodiments thereof, or a pharmaceutically acceptable salt thereof, and a pharmaceutically acceptable carrier.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydronaphthalene is an example of a 10-membered cycloalkyl group.

For compounds of the disclosure in which a variable appears more than once, each variable can be a different moiety independently selected from the group defining the variable. For example, where a structure is described having two R groups that are simultaneously present on the same compound, the two R groups can represent different moieties independently selected from the group defined for R.

The phrase “optionally substituted” means unsubstituted or substituted.

The term “substituted” means that an atom or group of atoms formally replaces hydrogen as a “substituent” attached to another group. The term “substituted” refers, unless otherwise indicated, to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. A single divalent substituent, e.g., oxo, can replace two hydrogen atoms.

The term “Cₙ₋ₘ” where n and m are integers is employed in combination with a chemical group to designate a range of the number of carbon atoms in the chemical group, with n and m defining the range. For example, C₁₋₆ alkyl refers to an alkyl group having 1, 2, 3, 4, 5, or 6 carbon atoms. The term is intended to include each and every member in the indicated range. Thus, Cₙ₋ₘ includes each member in the series Cₙ, Cₙ₊₁, . . . Cₘ₋₁, and Cₘ. Examples include C₁₋₄ (which includes C₁, C₂, C₃, and C₄), C₁₋₆ (which includes C₁, C₂, C₃, C₄, C₅, and C₆) and the like.

The term “alkyl,” employed alone or in combination with other terms, refers to a saturated hydrocarbon group that may be straight-chain or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like. In some embodiments, the alkyl group is methyl, ethyl, or propyl.

The term “alkylene,” employed alone or in combination with other terms, refers to a divalent alkyl linking group, which may be straight-chain or branched. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkenyl,” employed alone or in combination with other terms, refers to a straight-chain or branched hydrocarbon group corresponding to an alkyl group having one or more carbon-carbon double bonds. The term “Cₙ₋ₘ alkylenyl” refers to an alkenyl group having n to m carbon atoms. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. In some embodiments, the alkyl group has 1 to 6, 1 to 4, or 1 to 3 carbon atoms. Example alkoxy groups include methoxy, ethoxy, propoxy (e.g., n-propoxy and isopropoxy), t-butoxy, and the like. In some embodiments, alkoxy is methoxy.

The term “amino,” employed alone or in combination with other terms, refers to NH₂.

The term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

The term “C₁₋₃ alkoxy-C₁₋₃ alkyl” refers to a group of formula —(C₁₋₃ alkylene)-(C₁₋₃ alkoxy).

The term “C₁₋₃ alkoxy-C₁₋₃ alkoxy” refers to a group of formula —(C₁₋₃ alkoxylene)-(C₁₋₃ alkoxy).

The term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

The term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

The term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

The term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

The term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

The term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

The term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

The term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

The term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

The term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

The term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

The term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

The term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

The term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

The term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

The term “aminosulfonyl” refers to a group of formula —S(O)₂NH₂.

The term “aminosulfonylamino” refers to a group of formula —NHS(O)₂NH₂.

The term “aminocarbonylamino”, employed alone or in combination with other terms, refers to a group of formula —NHC(O)NH₂.

The term “carbonyl,” employed alone or in combination with other terms, refers to a —C(═O)— group.

The term “carboxy” refers to a group of formula —C(O)OH.

The term “cyano” or “nitrile” refers to a group of formula —C≡N, which also may be written as —CN.

The term “cyano-C₁₋₃ alkyl” refers to a group of formula —(C₁₋₃ alkylene)-CN.

The term “halo” or “halogen”, employed alone or in combination with other terms, refers to fluoro, chloro, bromo, and iodo. In some embodiments, “halo” refers to a halogen atom selected from F, Cl, or Br. In some embodiments, halo is F or Cl. In some embodiments, halo is F.

The term “haloalkyl,” employed alone or in combination with other terms, refers to an alkyl group in which one or more of the hydrogen atoms has been replaced by a halogen atom, having up to the full valency of halogen atom substituents, which may either be the same or different. In some embodiments, the halogen atoms are fluoro atoms. The term “Cₙ₋ₘ haloalkyl” refers to a Cₙ₋ₘ alkyl group having n to m carbon atoms and from at least one up to {2 (n to m)+1}halogen atoms, which may either be the same or different. In some embodiments, the alkyl group has 1 to 6, 1 to 4, or 1 to 3 carbon atoms. Example haloalkyl groups include CF₃, C₂F₅, CHF₂, CCl₃, CHCl₂, C₂Cl₅, and the like. In some embodiments, the haloalkyl group is a fluoroalkyl group.

The term “haloalkoxy,” employed alone or in combination with other terms, refers to a group of formula —O-(haloalkyl). The term “Cₙ₋ₘ haloalkoxy” refers to a haloalkoxy group, the haloalkyl group of which has n to m carbons. In some embodiments, the alkyl group has 1 to 6, 1 to 4, or 1 to 3 carbon atoms. An example haloalkoxy group is —OCF₃.

The term “H₂N—C₁₋₃ alkyl” refers to a group of formula —(C₁₋₃ alkylene)-NH₂.

The term “HO—C₁₋₃ alkoxy” refers to a group of formula —(C₁₋₃ alkoxylene)-OH.

The term “HO—C₁₋₃ alkyl” refers to a group of formula —(C₁₋₃ alkylene)-OH.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “thio” refers to a group of formula —SH.

The term “alkylthio,” employed alone or in combination with other terms, refers to a group of formula —S-alkyl. The term “Cₙ₋ₘ alkylthio” refers to a group of formula —S-alkyl, wherein the alkyl group has n to m carbon atoms. In some embodiments, the alkyl group has 1 to 6, 1 to 4 carbon, or 1 to 3 carbon atoms.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl.

In some embodiments, the aryl group is naphthyl.

The term “heteroaryl” or “heteroaromatic” employed alone or in combination with other terms, refers to a monocyclic or polycyclic (e.g., having 2 or 3 fused rings) aromatic hydrocarbon moiety, having one or more heteroatom ring members independently selected from nitrogen, sulfur and oxygen. In some embodiments, the heteroaryl group is a monocyclic or bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl, 2H-pyrazolo[4,3-c]pyridinyl, 1H-pyrazolo[3,4-c]pyridinyl, pyridinyl, 3H-imidazo[4,5-c]pyridinyl, 1,6-naphthyridinyl, 2,6-naphthyridinyl, 7H-purinyl, imidazo[1,5-a]pyrazinyl, imidazo[1,5-a]pyrazinyl, pyrazolo[1,5-a]pyrazinyl, imidazo[1,2-c]pyrimidinyl, 1H-pyrazolo[4,3-c]pyridinyl, 1H-imidazolyl, 3H-imidazo[4,5-b]pyridinyl, [1,2,4]triazolo[4,3-a]pyridinyl, pyrido[2,3-d]pyrimidinyl, 1,8-naphthyridinyl, 3a,7a-dihydro-1H-pyrazolo[3,4-d]pyrimidinyl, 1H-imidazo[4,5-c]pyridinyl, pyridin-2(1H)-onyl, or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments the heteroaryl group is a 5 to 10 membered heteroaryl group. In another embodiment the heteroaryl group is a 5 to 6 membered heteroaryl group. In some embodiments, the heteroaryl is a 5-6 membered heteroaryl moiety having carbon and 1, 2, or 3 heteroatoms independently selected from N, O and S. In some embodiments, the heteroaryl is a 5-10 membered heteroaryl moiety having carbon and 1, 2, or 3 heteroatoms independently selected from N, O and S. In some embodiments, the heteroaryl has 5-6 ring atoms and 1 or 2 heteroatom ring members independently selected from nitrogen, sulfur and oxygen. In some embodiments, no more than 2 heteroatoms of a 5-membered heteroaryl moiety are N.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, pyridazinyl, and pyridin-2(1H)-onyl.

A nine-membered heteroaryl ring is a heteroaryl group having nine ring atoms wherein one or more (e.g., 1, 2 or 3) ring atoms are independently selected from N, O and S. Exemplary nine-membered ring heteroaryls include benzofuran, benzo[b]thiophene, 1H-indole, 1H-benzo[d]imidazolyl, benzo[d]oxazolyl, benzo[d]thiazolyl, 1H-pyrrolo[3,2-b]pyridinyl, 1H-imidazo[4,5-b]pyridinyl, 1H-pyrrolo[3,2-c]pyridinyl, 1H-imidazo[4,5-c]pyridinyl, 1H-pyrrolo[2,3-c]pyridinyl, 3H-imidazo[4,5-c]pyridinyl, 1H-pyrrolo[2,3-b]pyridinyl, 3H-imidazo[4,5-b]pyridinyl, imidazo[1,2-a]pyridinyl,imidazo[1,2-a]pyrimidinyl,pyrazolo[1,5-a]pyrimidinyl,[1,2,4]triazolo[1,5-a]pyrimidinyl,[1,2,4]triazolo[1,5-a]pyridinyl,imidazo[1,2-c]pyrimidinyl,pyrrolo[1,2-a]pyrimidinyl, and 2H-pyrazolo[3,4-c]pyridinyl.

A ten−membered heteroaryl ring is a heteroaryl group having ten ring atoms wherein one or more (e.g., 1, 2 or 3) ring atoms are independently selected from N, O and S. Exemplary ten−membered ring heteroaryls are 1,7-naphthyridinyl, 2,7-naphthyridinyl, 3,7-naphthyridinyl, and 4,7-naphthyridinyl.

The term “cycloalkyl” or “cycloalkane” employed alone or in combination with other terms, refers to a non-aromatic cyclic hydrocarbon including cyclized alkyl and alkenyl groups. The terms “Cₙ₋ₘ cycloalkyl” refers to a cycloalkyl that has from n to m ring member carbon atoms. Cycloalkyl groups can include mono- or polycyclic (e.g., having 2, 3, or 4 fused, bridged, or spiro rings) ring systems. Also included are moieties that have one or more aromatic rings (e.g., aryl or heteroaryl rings) fused to (i.e., having a bond in common with) the cycloalkyl ring, for example, benzo derivatives of cyclopentane, cyclohexene, cyclohexane, and the like, or pyrido derivatives of cyclopentane or cyclohexane. A cycloalkyl group containing a fused aromatic ring can be attached through any ring-forming atom including a ring-forming atom of the fused aromatic ring. Ring-forming carbon atoms of a cycloalkyl group can be optionally substituted by oxo. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members, or 3 to 6 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. In some embodiments, the cycloalkyl group is cyclopropyl or cyclohexyl.

The term “heterocycloalkyl,” or “heterocycloalkane” or employed alone or in combination with other terms, refers to a non-aromatic ring or ring system, which has at least one carbon atom ring member and at least one heteroatom ring member independently selected from nitrogen, sulfur, oxygen, and phosphorus, and which has 4-14 ring members, 4-10 ring members, 4-7 ring members, or 4-6 ring members. The ring may contain one or more alkylene, alkenylene or alkynylene groups as part of the ring structure. The term “n−m-membered heterocycloalkyl” where n and m are integers refer to a heterocycloalkyl ring or ring system containing from n to m ring-forming atoms. An n−m-membered heterocycloalkyl include from 1 to m−1 carbon atoms and from 1 to m−1 heteroatoms. The term “n−membered heterocycloalkyl” where n is an integer refers to a heterocycloalkyl ring or ring system containing from n to m ring-forming atoms.

Included within the term “heterocycloalkyl” are monocyclic 4-, 5-, 6- and 7-membered heterocycloalkyl. Heterocycloalkyl groups can include mono- or polycyclic (e.g., having 2, 3 or 4 fused, bridged, or spiro rings) or spirocyclic ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or 3 to 8 ring forming atoms.

In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, dihydropyran ring, tetrahydropyran ring, tetrahyropyridine, azetidine ring, or tetrahydrofuran ring. In some embodiments, the heterocycloalkyl is a 4-7 membered heterocycloalkyl moiety having carbon and 1, 2, or 3 heteroatoms independently selected from N, O and S. In some embodiments, the heterocycloalkyl is 4-10 membered heterocycloalkyl moiety having carbon and 1, 2, or 3 heteroatoms independently selected from N, O and S.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Resolution of racemic mixtures of compounds can be carried out by methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the disclosure have the (R)-configuration. In other embodiments, the compounds have the (S)-configuration. In compounds with more than one chiral center, each of the chiral centers in the compound may be independently (R) or (S), unless otherwise indicated. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. Compounds herein identified by name or structure as one particular tautomeric form are intended to include other tautomeric forms unless otherwise specified (e.g., in the case of purine rings, unless otherwise indicated, if a compound name or structure described the 9H tautomer, it would be understood that the 7H tautomer is also encompassed).

The term, “compound,” is intended to include all stereoisomers, geometric iosomers, tautomers, and isotopes of the structures depicted. The term is also meant to refer to compounds of the disclosure, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., in the form of hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the disclosure, or salts thereof, are substantially isolated. By “substantially isolated” is meant that the compound is at least partially or substantially separated from the environment in which it was formed or detected. Partial separation can include, for example, a composition enriched in the compounds of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol, or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19 and in P. H. Stahl et al., Handbook of Pharmaceutical Salts: Properties, Selection, and Use, 2^{nd} Ed. (Wiley, 2011).

### III. Synthesis

As will be appreciated by those skilled in the art, the compounds provided herein, including salts and stereoisomers thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in P. Kocienski, Protecting Groups, 3^{rd} Ed. (Thieme, 2005); J. Robertson, Protecting Group Chemistry, (Oxford University Press, 2000); M. B. Smith et al., March's Advanced Organic Chemistry: Reactions, Mechanisms, and Structure, 8^{th} Ed. (Wiley, 2020); S. Petursson, J. Chem. Educ., 1997, 74(11), 1297-303; and P. G. M. Wuts et al., Greene's Protective Groups in Organic Synthesis, 5^{th} Ed., (Wiley, 2014).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as HPLC or TLC.

Compounds can be purified by those skilled in the art by a variety of methods, including HPLC (K. F. Blom, et al., J. Combi. Chem. 2004, 6(6), 874-83) and normal phase silica chromatography.

The Schemes below provide general guidance in connection with preparing the compounds of the invention. One skilled in the art would understand that the preparations shown in the Schemes can be modified or optimized using general knowledge of organic chemistry to prepare various compounds of the invention.

The preparation of compounds of formula (I) can be achieved through the process shown in Scheme 1, in which compounds of formula 1-1 undergo nucleophilic aromatic substitution with compounds of formula 1-2 in the presence of base (e.g., DIPEA). For compounds in which the A ring contains an NBoc group, final compounds (I) can be obtained through subsequent deprotection of the Boc group under acidic conditions (e.g., TFA) followed by functionalization of the free amine.

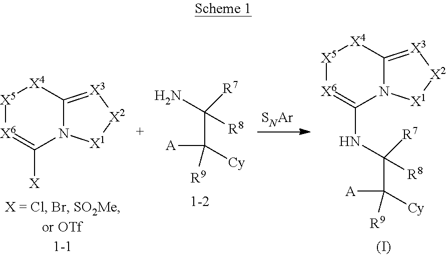

Compounds of formula 1-1 (X⁴═N, X⁵═CR⁵, and X⁶═CR⁶) can be prepared through the two step sequence shown in Scheme 2. Under acidic conditions (e.g., TFA in DMSO or HOAc in EtOH), compounds of formula 2-1 undergo cyclization with compounds 2-2 to give adducts 2-3 (P. J. Choi, et al., Tetrahedron Lett., 2022, 90, 153611). Cyclized compounds 2-3 can then be activated (e.g., POCl₃ or Tf₂O and DIPEA) (P. J. Choi, et al., Tetrahedron Lett., 2022, 90, 153611) to deliver compounds 1-1.

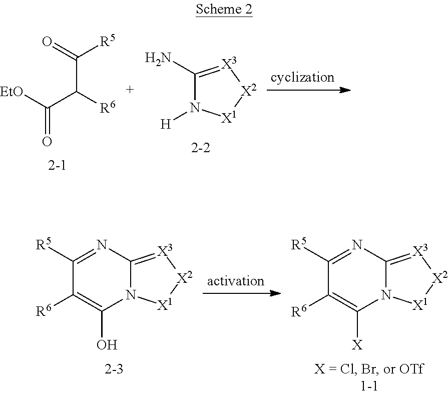

Compounds of formula 1-2 can be synthesized as shown in Scheme 3. Commercially available compounds 3-1 can be alkylated under basic conditions (e.g., NaHMDS) with alkyl halides 3-2 to give compounds 3-3, which can undergo a second alkylation under basic conditions (e.g., NaHMDS) with alkyl halides 3-4 to deliver compounds 3-5. The final compounds 1-2 can then be obtained through reduction of the nitrile to the amine (e.g., NiCl₂·6H₂O/NaBH₄ in MeOH) (S. Caddick, et al., Tetrahedron 2003, 59(29), 5417-5423).

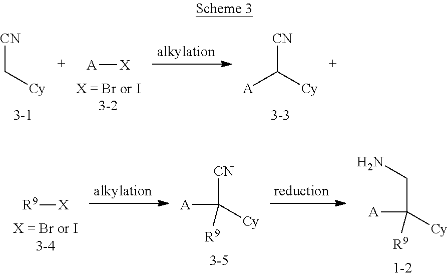

For the synthesis of particular compounds, the general schemes described above and specific methods described herein for preparing particular compounds can be modified. For example, the products or intermediates can be modified to introduce particular functional groups. Alternatively, the substituents can be modified at any step of the overall synthesis by methods know to one skilled in the art, e.g., as described by R. C. Larock, et al., Comprehensive Organic Transformations: A Guide to Functional Group Preparations, 3^{rd} Ed. Vols. 1-4 (Wiley, 2018); A. R. Katritzky, et al. (Eds.), Comprehensive Organic Functional Group Transformations, Vols. 1-6 (Pergamon Press, 1995), and A. R. Katritzky et al. (Eds.), Comprehensive Organic Functional Group Transformations II, Vols. 1-6 (Elsevier, 2^{nd} Edition, 2005);

Starting materials, reagents and intermediates whose synthesis is not expressly described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St.C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022).

### IV. Uses of the Compounds

Compounds of the present disclosure, including the compounds of Formula (I), or any of the embodiments thereof, are useful for therapy as described in further detail below. The present disclosure provides compounds of Formula (I), for use as a medicament, or for use in medicine, as described in further detail below. The present disclosure also provides the use of compounds of Formula (I), or any of the embodiments thereof, as a medicament, or for treating disease, as described in further detail below. The present disclosure also provides the use of compounds of Formula (I), or any of the embodiments thereof, in the manufacture of medicament for treating disease, as described in further detail below.

Compounds of the present disclosure can modulate, antagonize or inhibit the activity of the MRGPRX2 protein. As MRGPRX2 modulators, antagonists or inhibitors, the compounds of the disclosure are useful in the treatment of MRGPRX2 dependent conditions.

The compounds of the present disclosure may be used for treating an MRGPRX2 dependent condition caused by IgE independent activation of MRGPRX2 and that would benefit from modulating MRGPRX2. IgE independent activation of MRGPRX2 is capable of inducing mast cell degranulation and release of inflammatory mediators.

In some embodiments, the MRGPRX2 dependent condition is an itch associated condition, a pain associated condition, a pseudo-allergic reaction, an autoimmune or inflammatory disorder, or cancer-associated condition.

In some embodiments, the MRGPRX2 dependent condition is an itch associated condition, such as chronic itch; senile itch; contact dermatitis; allergic blepharitis; anaphylaxis; anaphylactoid drug reactions; anaphylactic shock; anemia; atopic dermatitis; bullous pemphigoid; candidiasis; chicken pox; end-stage renal failure; hemodialysis; cholestatic pruritus; chronic spontaneous urticaria; chronic inducible urticaria; contact dermatitis, dermatitis herpetiformis; diabetes; drug allergy, dry skin; dyshidrotic dermatitis; ectopic eczema; eosinophilic fasciitis; epidermolysis bullosa; erythrasma; food allergy; folliculitis; fungal skin infection; hemorrhoids; herpes; HIV infection; Hodgkin's disease; hyperthyroidism; iodinated contrast dye allergy; iron deficiency anemia; kidney disease; leukemia, porphyria; lymphoma; mast cell activation syndrome, malignancy; mastocystosis; multiple myeloma; neurodermatitis; onchocerciasis; Paget's disease; pediculosis; polycythemia rubra vera; prurigo nodularis; lichen planus; lichen sclerosis; pruritus ani; pseudo-allergic reactions; pseudorabies; psoriasis; rectal prolapse; sarcoidosis granulomas; scabies; schistosomiasis; scleroderma, severe stress, stasis dermatitis; swimmer's itch; thyroid disease; tinea cruris; uremic pruritus; rosacea; cutaneous amyloidosis; scleroderma; acne; wound healing; burn healing; ocular itch; and urticaria.

In some embodiments, the MRGPRX2 dependent condition is a pain associated condition, such as acute pain, advanced prostate cancer, AIDS-related pain, ankylosing spondylitis, arachnoiditis, arthritis, arthrofibrosis, ataxic cerebral palsy, autoimmune atrophic gastritis, avascular necrosis, back pain, Behcet's disease (syndrome), burning mouth syndrome, bursitis, cancer pain, carpal tunnel, cauda equina syndrome, central pain syndrome, cerebral palsy, cervical stenosis, Charcot-Marie-Tooth (CMT) disease, chronic fatigue syndrome (CFS), chronic functional abdominal pain (CFAP), chronic pain, chronic pancreatitis, chronic pelvic pain syndrome, collapsed lung (pneumothorax), complex regional pain syndrome (CRPS), reflex sympathetic dystrophy syndrome (RDS), corneal neuropathic pain, Crohn's disease, degenerative disc disease, dental pain, Dercum's disease, dermatomyositis, diabetic peripheral neuropathy (DPN), dystonia, Ehlers-Danlos syndrome (EDS), endometriosis, eosinophilia-myalgia syndrome (EMS), erythromelalgia, fibromyalgia, gout, headaches, herniated disc, hydrocephalus, intercostal neuralgia, interstitial cystitis, irritable bowel syndrome (IBS), juvenile dermatositis (dermatomyositis), knee injury, leg pain, loin pain-haematuria syndrome, lupus, Lyme disease, medullary sponge kidney (MSK), meralgia paresthetica, mesothelioma, migraine, musculoskeletal pain, myofascial pain, myositis, neck pain, neuropathic pain, occipital neuralgia, osteoarthritis, Paget's disease, pain crisis in sickle cell disease; Parsonage-Turner syndrome, pelvic pain, periodontitis pain, peripheral neuropathy, phantom limb pain, pinched nerve, polycystic kidney disease, polymyalgia rheumatica, polymyositis, porphyria, post herniorrhaphy pain syndrome, post mastectomy pain, postoperative pain, pain syndrome, post stroke pain, post thoracotomy pain syndrome, postherpetic neuralgia (shingles), post-polio syndrome, primary lateral sclerosis, psoriatic arthritis, pudendal neuralgia, radiculopathy, Raynaud's disease, rheumatoid arthritis (RA), sacroiliac joint dysfunction, sarcoidosis, Scheuermann's kyphosis disease, sciatica, scoliosis, shingles (herpes zoster), Sjögren's syndrome, spasmodic torticollis, sphincter of oddi dysfunction, spinal cerebellum ataxia (SCA ataxia), spinal cord injury, spinal stenosis, syringomyelia, Tarlov cysts, transverse myelitis, trigeminal neuralgia, neuropathic pain, ulcerative colitis, vascular pain and vulvodynia.

In some embodiments, the MRGPRX2 dependent condition is a pseudo-allergic reaction, such as pseudo-allergic reactions caused by secretagogues, cationic peptidergic drugs, anionic peptidergic drugs, neutral peptidergic drugs, non-steroidal anti-inflammatory drugs, neuropeptides, antimicrobial peptides, opioids, neuromuscular blocking agents, antidepressant agents, antipsychotic agents, antihistamine agents, antineoplastic agents, fluoroquinolone and non-fluoroquinolone antibiotics and tyrosine-kinase inhibitors. The phrase “pseudo-allergic reaction” refers to an IgE-independent allergic reaction, characterized by release of histamine and cytokines, activation of the complement system, atypical synthesis of eicosanoids, inflammation, skin flushing, headache, edema, hypotension, urticaria (hives), bronchospasm, or any combination thereof. A pseudo-allergic reaction is a hypersensitivity reaction manifested by systemic responses. The symptoms of pseudo-allergic reaction are identical to anaphylaxis, however their mechanism is non-IgE-mediated. A pseudo-allergic reaction may be caused by a range of cationic substances, collectively called basic secretagogues, including inflammatory peptides and drugs associated with allergic-type reactions. In one embodiment, the pseudo-allergic reaction is caused by MCD peptide, substance P, VIP, PACAP, dynorphin, somatostatin, Compound 48/80, cortistatin-14, mastoparan, melittin, cathelicidin peptides, ciprofloxacin, vancomycin, leuprolide, goserelin, histrelin, triptorelin, cetrorelix, ganirelix, degarelix, octreotide, lanreotide, pasireotide, sermorelin, tesamorelin, icatibant, glatiramer acetate, teriparatide, pramlintide, bleomycin, exenatide, glucagon, liraglutide, enfuvirtide, colistimethate, succinylcholine, tubocurarine, atracurium, mivacurium, and rocuronium.

In some embodiments, the MRGPRX2 dependent condition is an autoimmune disorder or inflammatory condition, such as chronic inflammation, mast cell activation syndrome, multiple sclerosis, Steven Johnson's syndrome, toxic epidermal necrolysis, appendicitis, bursitis, cutaneous lupus, colitis, cystitis, dermatitis, phlebitis, reflex sympathetic dystrophy/complex regional pain syndrome (RSD/CRPS), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, eosinophilic duodenitis, eosinophilic gastroenteritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, schwannomatosis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, biliary atresia, acute and chronic hepatitis B and C virus, liver tumors and cancer, alcoholic liver disease, polycystic liver disease, liver cholangiocarcinoma, primary sclerosing cholangitis, primary biliary cholangitis, neuromyelitis optica spectrum disorder, cardiovascular disease, inflammation induced by bacterial or viral infection, inflammation associated with SARS-COV-2 infection or its variants and coronavirus disease 2019 (COVID-19), acute respiratory distress syndrome, pneumonia, long/long-term/chronic COVID, postacute sequelae of COVID-19 (PASC), myalgic encephalomyelitis/chronic fatigue syndrome (ME/CFS “brain fog”) and vasculitis.

In some embodiments, the compounds of the disclosure are useful to treat a cancer/tumor associated condition, such as adenoid cystic carcinoma, adrenal gland tumor, amyloidosis, anal cancer, appendix cancer, astrocytoma, ataxia-telangiectasia, Beckwith-Wiedemann syndrome, cholangiocarcinoma, Birt-Hogg-Dubé syndrome, bone cancer, brain stem glioma, brain tumor, breast cancer (inflammatory, metastatic, male), prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), liver cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomatosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Wermer's syndrome and xeroderma pigmentosum.

The MRGPRX2 dependent condition may be selected from the group consisting of abdominal aortic aneurysms, acute contact dermatitis, allergic rhinitis, amyotrophic lateral sclerosis, asthma, atopic dermatitis, autism, cancer, chronic inducible urticaria, chronic itch, chronic obstructive pulmonary disease, chronic spontaneous urticaria, cold urticaria, contact urticaria, coronary artery disease, cough, Crohn's disease, deep vein thrombosis, drug-induced anaphylactic reactions, endometriosis, fibromyalgia, geographic atrophy, idiopathic chronic cough, idiopathic pulmonary fibrosis, inflammatory pain, interstitial cystitis, irritable bowel syndrome, mast cell activation syndrome, mastocytosis, metabolic syndrome, migraine, multiple sclerosis, nasal polyps, neurodermatitis, neuropathic itch, neuropathic pain, obesity, oesophageal reflux, osteoarthritis, periodontitis, prurigo nodularis, pruritus, pseudo-anaphylaxis, psoriasis, rheumatoid arthritis, rosacea, seborrheic dermatitis, sickle cell disease, ulcerative colitis, and ulcers.

The MRGPRX2 dependent condition may be selected from the group consisting of autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjögren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

The MRGPRX2 dependent condition may be selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticaria, axonal or neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's granulomatosis), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, Immunoregulatory lipoproteins, inclusion body myositis, interstitial cystitis, juvenile arthritis, juvenile diabetes (type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, subacute bacterial endocarditis (SBE), Susac's syndrome, sympathetic ophthalmia, takayasu's arteritis, temporal arteritis/giant cell arteritis, thrombocytopenic purpura (TTP), tolosa-hunt syndrome, transverse myelitis, type 1 diabetes, ulcerative colitis, undifferentiated connective tissue disease (UCTD), uveitis, vasculitis, vesiculobullous dermatosis, vitiligo, or Wegener's granulomatosis (i.e., granulomatosis with polyangiitis (GPA)).

The present disclosure provides a method of treating a disease in a patient. The disease can be a MRGPRX2 dependent condition, including any of the MRGPRX2 dependent conditions described herein. The method comprises administering to the patient in need of the treatment a therapeutically effective amount of a compound of Formula (I), or any of the embodiments thereof. The condition treated can be any of the conditions described herein.

The phrase “MRGPRX2 dependent condition” refers to a condition in which the activation, over sensitization, or desensitization of MRGPRX2 by a natural or synthetic ligand initiates, mediates, sustains, or augments a pathological condition. For example, it is known that some cationic peptidergic drugs cause pseudo-allergic reactions in patients. MRGPRX2 is sensitive to (or activated by) secretagogues, cationic peptidergic drugs, including icatibant, leuprolide, or ganirelix, neutral and anionic peptidergic drugs (e.g., exenatide, glucagon, liraglutide, enfuviritide, colistimethate), neuromuscular blocking agents (atracurium mivacurium), non-steroidal anti-inflammatory drugs, neuropeptides, antimicrobial peptides. Moreover, overexpression of MRGPRX2 and/or overactivity of MRGPRX2 may also render mast cells more susceptible to activation by endogenous and/or exogenous ligands. Without being bound by theory, it is to be understood that by modulating MRGPRX2, pseudo-allergic reactions, itch, pain, inflammatory and autoimmune disorders can be eased.

The term “autoimmune disorder”, or “inflammatory disorder” means a disease or disorder arising from and/or directed against an individual's own tissues or organs, or a co-segregate or manifestation thereof, or resulting condition therefrom. typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues.

The phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch).

The term “administration” refers to providing a compound, or a pharmaceutical composition comprising the compound as described herein. The compound or composition can be administered by another person to the subject or it can be self-administered by the subject. Non-limiting examples of routes of administration are oral, parenteral (e.g., intravenous), or topical.

The term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “contacting” refers to the bringing together of indicated moieties in an in vitro system or an in vivo system. For example, “contacting” the MRGPRX2 with a compound described herein includes the administration of a compound described herein to an individual or patient, such as a human, having MRGPRX2, as well as, for example, introducing a compound described herein into a sample containing a cellular or purified preparation containing the MRGPRX2.

The term “individual” or “patient,” used interchangeably, refers to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

The phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

The Federal Food, Drug, and Cosmetic Act defines “pediatric” as a subject aged 21 or younger at the time of their diagnosis or treatment. Pediatric subpopulations are further characterized as: (i) neonates—from birth through the first 28 days of life; (ii) infants—from 29 days to less than 2 years; (iii) children—2 years to less than 12 years; and (iv) adolescents—aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### V. Combination Therapies

One or more additional pharmaceutical agents or treatment methods can be used in combination with compounds described herein for treatment of MRGPRX2 dependent conditions, as described herein. The agents can be combined with the present compounds in a single dosage form, or the agents can be administered simultaneously or sequentially as separate dosage forms.

In some embodiments, the additional therapeutic agent is an antihistamine, such as an H1 receptor antagonist or an H2 receptor antagonist. In one embodiment, the additional therapeutic agent is an H1 receptor antagonist antihistamine, such as levocetirizine, loratadine, fexofenadine, cetirizine, desloratadine, olopatadine, diphenhydramine, cyproheptadine, hydroxyzine pamoate or ketotifen. In one embodiment, the additional therapeutic agent is a H2 receptor antagonist, such as cimetidine, nizatidine, ranitidine or famotidine. In one embodiment, the additional therapeutic agent is a leukotriene receptor antagonist or leukotriene synthesis inhibitor, such as montelukast, zafirlukast, pranlukast, or 5-lipoxygenase inhibitor (e.g., zileuton, Hypericum perforatum). In one embodiment, the additional therapeutic agent is an immunomodulatory agent such as omalizumab or immunoglobulin therapy. In one embodiment, the additional therapeutic agent is a corticosteroid, such as hydrocortisone, cortisone, betamethasone, triamcinolone, prednisone, prednisolone, or fludrocortisone. In one embodiment, the additional therapeutic agent is a tricyclic antidepressant that can relieve itch such as doxepin, amitriptyline or nortriptyline. In one embodiment, the additional therapeutic agent is an anti-inflammatory drug such as dapsone, sulfasalazine, hydroxychloroquine or colchicine. In one embodiment, the additional therapeutic agent is an immunosuppressant such as cyclosporine, methotrexate, mycophenolic acid or tacrolimus.

### VI. Pharmaceutical Formulations and Dosage Forms

When employed as pharmaceuticals, compounds described herein can be administered in the form of pharmaceutical compositions which refers to a combination of one or more compounds described herein, and at least one pharmaceutically acceptable carrier or excipient. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more compounds described herein in combination with one or more pharmaceutically acceptable carriers or excipients. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is suitable for topical administration.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from, for example, about 5 mg to about 1000 mg, about 5 mg to about 100 mg, about 100 mg to about 500 mg, or about 10 to about 30 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid pre-formulation composition containing a homogeneous mixture of one or more compounds described herein. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds, or compositions as described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of the compounds in a pharmaceutical composition can vary depending upon a number of factors including dosage, chemical characteristics (e.g., hydrophobicity), and the route of administration. For example, compounds of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Compounds described herein can also be formulated in combination with one or more additional active ingredients, which can include any pharmaceutical agent such as anti-viral agents, vaccines, antibodies, immune enhancers, immune suppressants, anti-inflammatory agents and the like.

### VII. Labelled Compounds

Compounds of the disclosure also include all isotopes of atoms occurring in the intermediates or final compounds. Isotopes include those atoms having the same atomic number but different mass numbers.

The present disclosure further includes isotopically-labelled compounds of the disclosure. An “isotopically-labelled” is a compound of the disclosure where one or more atoms are replaced or substituted by an atom having an atomic mass or mass number different from the atomic mass or mass number typically found in nature (i.e., naturally occurring). A “radio-labelled” compound is an isotopically-labelled compound in which one or more atoms are replaced or substituted by an atom of an isotope that is radioactive.

Suitable isotopes that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated. The symbol D included in a chemical formula or as a substituent indicates that deuterium is incorporated in the position labelled at greater than natural abundance, and typically indicates an abundance of equal to or greater than 50%, preferably equal to or greater than 90% or equal to or greater than 95%, 96%, 97%, 98%, 99%, 99.5%, 99.9%, 99.95%, or 99.99% relative to all forms of hydrogen.

One or more constituent atoms of the compounds presented herein can be replaced or substituted with isotopes of the atoms in natural or non-natural abundance. In some embodiments, the compound includes at least one deuterium atom. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms.

Synthetic methods for including isotopes into organic compounds are known in the art (A. F. Thomas, Deuterium Labeling in Organic Chemistry, (Appleton-Century-Crofts, New York, N.Y., 1971); J. Atzrodt, et al., Angew. Chem. Int. Ed., 2007, 7744-65; J. R. Hanson, The Organic Chemistry of Isotopic Labelling, (Royal Society of Chemistry, 2011)). Isotopically labelled compounds can used in various studies such as NMR spectroscopy, metabolism experiments, and/or assays.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes, et al., J. Med. Chem. 2011, 54(1), 201-10; R. Xu et al., J. Label. Compd. Radiopharm. 2015, 58, 308-12).

The radionuclide that is incorporated in the instant radio-labelled compounds will depend on the specific application of that radio-labelled compound. For example, for in vitro adenosine receptor labeling and competition assays, compounds that incorporate ³H, ¹⁴C, ⁸²Br, ¹²⁵I, ¹³¹I or ³S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labelled” or “labelled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

### VIII. Kits

The present disclosure also includes pharmaceutical kits useful, for example, in the treatment of MRGPRX2 dependent conditions, as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in detail by way of specific examples. The following examples are offered for illustrative purposes, and are not intended to limit the invention in any manner. Those of skill in the art will readily recognize a variety of non-critical parameters which can be changed or modified to yield essentially the same results.

Experimental procedures for compounds of the disclosure are provided below. Preparatory LCMS purifications of some of the compounds prepared were performed on Waters mass-directed fractionation systems. The basic equipment setup, protocols, and control software for the operation of these systems have been described in detail in the literature. See e.g., K. F. Blom, J. Combi. Chem., 2002, 4(4), 295-301; K. F. Blom, et al., J. Combi. Chem., 2003, 5(5), 670-683; and K. F. Blom, et al., J. Combi. Chem. 2004, 6(6), 874-83. The compounds separated were typically subjected to analytical LCMS for purity analysis under the following conditions: Instrument; Agilent 1100 series, LC/MSD, Column: Waters SUNFIRE® C₁₈ 5 μm, 2.1×50 mm, Buffers: mobile phase A: 0.025% aq. TFA and mobile phase B: MeCN; gradient 2% to 80% of B in 3 min. with flow rate 2.0 mL/min.

Some of the compounds prepared were also separated on a preparative scale by RP-HPLC with MS detector or FCC (silica gel) as indicated in the Examples. Typical preparative RP-HPLC column conditions are as follows:

pH=2 purifications: Waters SUNFIRE® C₁₈ 5 μm, 19×100 mm column, eluting with mobile phase A: 0.1% aq. TFA and mobile phase B: MeCN; the flow rate was 30 mL/min., the separating gradient was optimized for each compound using the Compound Specific Method Optimization protocol as described in the literature [see K. F. Blom, et al., J. Combi. Chem. 2004, 6(6), 874-83]. Typically, the flow rate used with a 30×100 mm column was 60 mL/min. pH=10 purifications: Waters XBRIDGE® C₁₈ 5 μm, 19×100 mm column, eluting with mobile phase A: 0.15% aq. NH₄OH and mobile phase B: MeCN; the flow rate was 30 mL/min., the separating gradient was optimized for each compound using the Compound Specific Method Optimization protocol as described in the literature [See K. F. Blom, et al., J. Combi. Chem. 2004, 6(6), 874-83]. Typically, the flow rate used with a 30×100 mm column was 60 mL/min.

### Example 1. 3-(1-((2,5-Bis(trifluoromethyl)pyrazolo[1,5-a]pyrimidin-7-yl)amino)-2-(4-fluorophenyl)butan-2-yl)azetidine-1-carboxamide

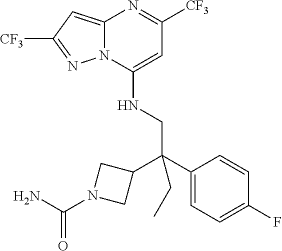 [SMILES: CCC(CNc1cc(C(F)(F)F)nc2cc(C(F)(F)F)nn12)(c1ccc(F)cc1)C1CN(C(N)=O)C1]

### Step 1. 2,5-Bis(trifluoromethyl)pyrazolo[1,5-a]pyrimidin-7-ol

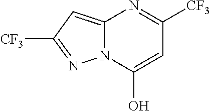 [SMILES: Oc1cc(C(F)(F)F)nc2cc(C(F)(F)F)nn12]

To a stirred solution of 3-(trifluoromethyl)-1H-pyrazol-5-amine (5.0 g, 33.1 mmol) and ethyl 4,4,4-trifluoro-3-oxobutanoate (4.8 mL, 33.1 mmol) in DMSO (35 mL), TFA (0.51 mL, 6.6 mmol) was added and then the solution was stirred at r.t. for 2 days. After this time, the reaction mixture was poured into 50 mL of ice water, stirred for 30 min., and then filtered to give a crude solid. The dried solid was then purified by FCC (BIOTAGE® ISOLERA™, pure hexanes to 30% EtOAc) to give the sub-title compound as a white solid. LCMS calc. for C₈H₄F₆N₃O (M+H)⁺: m/z=272.0; found: 272.0.

### Step 2. 7-Chloro-2,5-bis(trifluoromethyl)pyrazolo[1,5-a]pyrimidine

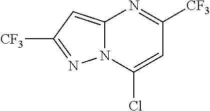 [SMILES: FC(F)(F)c1cc(Cl)n2nc(C(F)(F)F)cc2n1]

A solution of 2,5-bis(trifluoromethyl)pyrazolo[1,5-a]pyrimidin-7-ol (2.0 g, 7.38 mmol), POCl₃ (6.88 mL, 73.8 mmol) and DIPEA (1.3 mL, 7.38 mmol) in toluene (7 mL) was stirred at 100° C. for 2 h. After this time, the reaction mixture was allowed to cool to r.t. and poured into 500 mL of ice water. The mixture was stirred vigorously for 1 h, and then the precipitated solid was filtered out, washed with more water (3×5 mL), and dried under high vacuum overnight to give a brown solid which was used directly in Step 5. LCMS calc. for C₈H₃ClF₆N₃(M+H)⁺: m/z=290.0; found: 290.0.

### Step 3. tert-Butyl 3-(1-cyano-1-(4-fluorophenyl)propyl)azetidine-1-carboxylate

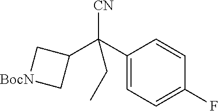 [SMILES: CCC(C#N)(c1ccc(F)cc1)C1CN(C(=O)OC(C)(C)C)C1]

To a solution of 2-(4-fluorophenyl)acetonitrile (0.444 mL, 3.70 mmol) in THE (10 mL) under nitrogen, NaHMDS (3.7 mL, 3.7 mmol, 1.0 M in THF) was added all at once. The solution was stirred at r.t. for 20 min., and then tert-butyl 3-iodoazetidine-1-carboxylate (1.05 g, 3.70 mmol) was added dropwise in THF (6 mL) over 45 min. The solution was stirred at r.t. for another 1.5 h, and then NaHMDS (3.7 mL, 3.7 mmol, 1.0 M in THF) was added all at once. The solution was stirred at r.t. for 20 min., and then iodoethane (0.3 mL, 3.70 mmol) was added all at once. The solution was stirred at r.t. overnight, and then the mixture was diluted with EtOAc and washed with water and brine. The organic phase was dried (Na₂SO₄), filtered, concentrated, and purified by FCC (BIOTAGE® ISOLERA™, pure hexanes to 20% EtOAc) to give the sub-title compound as a yellow oil. LCMS calc. for C₁₄H₁₆FN₂O₂(M+H-t-Bu)⁺: m/z=263.1; found: 263.1.

### Step 4. tert-Butyl 3-(1-amino-2-(4-fluorophenyl)butan-2-yl)azetidine-1-carboxylate

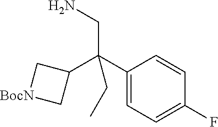 [SMILES: CCC(CN)(c1ccc(F)cc1)C1CN(C(=O)OC(C)(C)C)C1]

To a stirred solution of tert-butyl 3-(1-cyano-1-(4-fluorophenyl)propyl)azetidine-1-carboxylate (700 mg, 2.2 mmol) and nickel(II) chloride hexahydrate (523 mg, 2.2 mmol) in 10 mL of MeOH, NaBH₄ (250 mg, 6.60 mmol) was added portionwise over 10 min. The solution was stirred at r.t. for another 2 h, and then was diluted with 200 mL of water and extracted with EtOAc (2×500 mL). The combined organic phases were dried (Na₂SO₄), filtered, concentrated, and purified by FCC (BIOTAGE® ISOLERA™, pure DCM to 10% MeOH) to give the sub-title compound as a colorless oil. LCMS calc. for C₁₈H₂₈FN₂O₂(M+H)⁺: m/z=323.2; found: 323.2.

### Step 5. N-(2-(Azetidin-3-yl)-2-(4-fluorophenyl)butyl)-2,5-bis(trifluoromethyl)pyrazolo[1,5-a]pyrimidin-7-amine

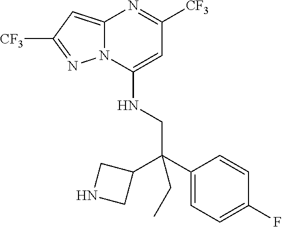 [SMILES: CCC(CNc1cc(C(F)(F)F)nc2cc(C(F)(F)F)nn12)(c1ccc(F)cc1)C1CNC1]

A solution of 7-chloro-2,5-bis(trifluoromethyl)pyrazolo[1,5-a]pyrimidine (85 mg, 0.294 mmol), tert-butyl 3-(1-amino-2-(4-fluorophenyl)butan-2-yl)azetidine-1-carboxylate (118 mg, 0.367 mmol), and DIPEA (0.154 mL, 0.881 mmol) in DMSO (1.5 mL) was heated at 110° C. for 2 h. After this time, the mixture was allowed to cool to r.t., diluted with EtOAc, and washed with water and brine. The organic phase was dried (Na₂SO₄), filtered, and concentrated to give a brown oil. The crude oil was taken up in DCM (1 mL) and TFA (1 mL) and stirred at r.t. for 1 h. After this time, the mixture was concentrated, diluted with EtOAc, and washed sat. aq. NaHCO₃ and water. The organic phase was collected, dried (Na₂SO₄), filtered, and concentrated to give a brown oil which was used directly in the next step. LCMS calc. for C₂₁H₂₁F₇N₅ (M+H)⁺: m/z=476.2; found: 476.3.

### Step 6. 3-(1-((2,5-Bis(trifluoromethyl)pyrazolo[1,5-a]pyrimidin-7-yl)amino)-2-(4-fluorophenyl)butan-2-yl)azetidine-1-carboxamide

A solution of N-(2-(azetidin-3-yl)-2-(4-fluorophenyl)butyl)-2,5-bis(trifluoromethyl)pyrazolo[1,5-a]pyrimidin-7-amine (70 mg, 0.147 mmol), phenyl carbamate (20 mg, 0.147 mmol) and DIPEA (0.13 mL, 0.736 mmol) in MeCN (1 mL) was heated at 55° C. for 1 h. After this time, the mixture was diluted with MeCN and purified by prep.-LCMS (Waters SUNFIRE® C₁₈ column, eluting with a gradient of MeCN/water containing 0.1% TFA, at a flow rate of 60 mL/min) to afford the title compound. LCMS calc. for C₂₂H₂₂F₇N₆O (M+H)⁺: m/z=519.2; found: 519.2.

The title compound was prepared as a racemic mixture. The racemic mixture is separable into its enantiomers (R)-3-(1-((2,5-bis(trifluoromethyl)pyrazolo[1,5-a]pyrimidin-7-yl)amino)-2-(4-fluorophenyl)butan-2-yl)azetidine-1-carboxamide and (S)-3-(1-((2,5-bis(trifluoromethyl)pyrazolo[1,5-a]pyrimidin-7-yl)amino)-2-(4-fluorophenyl)butan-2-yl)azetidine-1-carboxamide.

### Example 2. 4-(1-((2,5-Bis(trifluoromethyl)pyrazolo[1,5-a]pyrimidin-7-yl)amino)-2-(4-fluorophenyl)propan-2-yl)piperidine-1-carboxamide

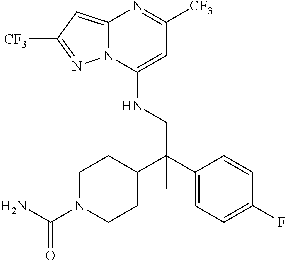 [SMILES: CC(CNc1cc(C(F)(F)F)nc2cc(C(F)(F)F)nn12)(c1ccc(F)cc1)C1CCN(C(N)=O)CC1]

### Step 1. tert-Butyl 4-(cyano(4-fluorophenyl)methyl)piperidine-1-carboxylate

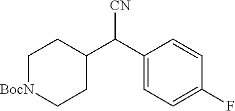 [SMILES: CC(C)(C)OC(=O)N1CCC(C(C#N)c2ccc(F)cc2)CC1]

In a vial, 2-(4-fluorophenyl)acetonitrile (800 mg, 5.92 mmol) was combined with THE (5 mL) under nitrogen and then NaHMDS (5.92 mL, 5.92 mmol, 1.0 M in THF) was added all at once. The solution was stirred at r.t. for 20 min., and then tert-butyl 4-iodopiperidine-1-carboxylate (1.84 g, 5.92 mmol) was added over about 5 min. The solution was stirred at r.t. for another 2 h, and then was diluted with EtOAc and washed with water and brine. The organic phase was dried (Na₂SO₄), filtered, concentrated, and purified by FCC (BIOTAGE® ISOLERA™, pure hexanes to 20% EtOAc) to give the sub-title compound as a yellow oil. LCMS calc. for C₁₄H₁₆FN₂O₂(M+H-t-Bu)⁺: m/z=263.1; found: 263.1.

### Step 2. tert-Butyl 4-(1-cyano-1-(4-fluorophenyl)ethyl)piperidine-1-carboxylate

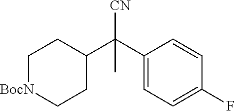 [SMILES: CC(C)(C)OC(=O)N1CCC(C(C)(C#N)c2ccc(F)cc2)CC1]

In a vial, tert-butyl 4-(cyano(4-fluorophenyl)methyl)piperidine-1-carboxylate (0.942 g, 2.96 mmol) was combined with THE (5 mL) under nitrogen and then NaHMDS (2.96 mL, 2.96 mmol, 1.0 M in TIF) was added all at once. The solution stirred at r.t. for 20 min., and then methyl iodide (0.185 mL, 2.96 mmol) was added over about 5 min. The solution was stirred at r.t. overnight, and then was diluted with EtOAc and washed with water and brine. The organic phase was dried (Na₂SO₄), filtered, concentrated, and purified by FCC (BIOTAGE© ISOLERA™, pure hexanes to 15% EtOAc) to give the sub-title compound as a yellow oil. LCMS calc. for C₁₅H₁₈FN₂O₂(M+H-t-Bu)⁺: m/z=277.3; found: 277.1.

### Step 3. tert-Butyl 4-(1-amino-2-(4-fluorophenyl)propan-2-yl)piperidine-1-carboxylate

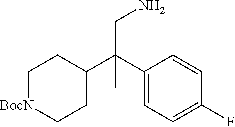 [SMILES: CC(C)(C)OC(=O)N1CCC(C(C)(CN)c2ccc(F)cc2)CC1]

To a stirred solution of tert-butyl 4-(1-cyano-1-(4-fluorophenyl)ethyl)piperidine-1-carboxylate (235 mg, 0.707 mmol) and nickel(II) chloride hexahydrate (202 mg, 0.848 mmol) in MeOH (20 mL), NaBH₄ (134 mg, 3.53 mmol) was added portionwise over about 15 min. The solution was stirred at r.t. for 2 h and then the mixture was diluted with 200 mL of water and extracted with EtOAc (2×500 mL). The combined organic phases were dried (Na₂SO₄), filtered, concentrated, and purified by FCC (BIOTAGE® ISOLERA™, pure DCM to 10% MeOH) to give the sub-title compound as a colorless oil. LCMS calc. for C₁₅H₂₂FN₂O₂(M+H-t-Bu)⁺: m/z=281.2; found: 281.2.

### Step 4. N-(2-(4-Fluorophenyl)-2-(piperidin-4-yl)propyl)-2,5-bis(trifluoromethyl)pyrazolo[1,5-a]pyrimidin-7-amine

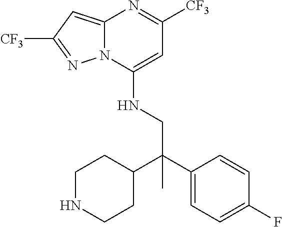 [SMILES: CC(CNc1cc(C(F)(F)F)nc2cc(C(F)(F)F)nn12)(c1ccc(F)cc1)C1CCNCC1]

A solution of 7-chloro-2,5-bis(trifluoromethyl)pyrazolo[1,5-a]pyrimidine (20 mg, 0.068 mmol), tert-butyl 4-(1-amino-2-(4-fluorophenyl)propan-2-yl)piperidine-1-carboxylate (23 mg, 0.068 mmol) (see Example 1, Step 2) and DIPEA (0.060 mL, 0.342 mmol) in DMSO (0.5 mL) was heated at 110° C. for 2 h. After this time, the mixture was allowed to cool to r.t., diluted with EtOAc, and washed with water and brine. The organic phase was dried over (Na₂SO₄), filtered, and concentrated to give a brown oil. The crude oil was taken up in DCM (0.5 mL) and TFA (0.5 mL) and stirred at r.t. for 1 h. After this time, the mixture was concentrated, diluted with EtOAc, and washed with sat. aq. NaHCO₃ and water. The organic phase was collected, dried (Na₂SO₄), filtered, and concentrated to give a brown oil which was used directly in the next step. LCMS calc. for C₂₂H₂₃F₇N₅ (M+H)⁺: m/z=490.2; found: 490.3.

### Step 5. 4-(1-((2,5-Bis(trifluoromethyl)pyrazolo[1,5-a]pyrimidin-7-yl)amino)-2-(4-fluorophenyl)propan-2-yl)piperidine-1-carboxamide

A solution of N-(2-(4-fluorophenyl)-2-(piperidin-4-yl)propyl)-2,5-bis(trifluoromethyl)pyrazolo[1,5-a]pyrimidin-7-amine (10 mg, 0.020 mmol), phenyl carbamate (3 mg, 0.020 mmol) and DIPEA (0.011 mL, 0.061 mmol) in MeCN (0.5 mL) was heated at 65° C. for 2 h. After this time, the mixture was diluted with MeCN and purified by prep.-LCMS (Waters SUNFIRE® C₁₈ column, eluting with a gradient of MeCN/water containing 0.1% TFA, at a flow rate of 60 mL/min) to afford the title compound. LCMS calc. for C₂₃H₂₄F₇N₆O (M+H)⁺: m/z=533.2; found: 533.2. ¹H NMR (500 MHz, DMSO) δ 7.97 (t, J=6.6 Hz, 1H), 7.45 (dd, J=8.7, 5.4 Hz, 2H), 7.12 (s, 1H), 7.04 (t, J=8.6 Hz, 2H), 6.39 (s, 1H), 4.08 (d, J=13.4 Hz, 1H), 3.98-3.86 (m, 2H), 3.80 (dd, J=14.0, 7.0 Hz, 1H), 2.66 (m, 1H), 2.57-2.48 (m, 1H), 2.07 (m, 1H), 1.90 (m, 1H), 1.32 (s, 3H), 1.22-1.07 (m, 2H), 0.98-0.87 (m, 1H).

The title compound was prepared as a racemic mixture. The racemic mixture is separable into its enantiomers (R)-4-(1-((2,5-bis(trifluoromethyl)pyrazolo[1,5-a]pyrimidin-7-yl)amino)-2-(4-fluorophenyl)propan-2-yl)piperidine-1-carboxamide and (S)-4-(1-((2,5-bis(trifluoromethyl)pyrazolo[1,5-a]pyrimidin-7-yl)amino)-2-(4-fluorophenyl)propan-2-yl)piperidine-1-carboxamide.

### Examples 3 and 4. 3-(1-((2,5-Bis(trifluoromethyl)pyrazolo[1,5-a]pyrimidin-7-yl)amino)-2-(4-fluorophenyl)propan-2-yl)pyrrolidine-1-carboxamide

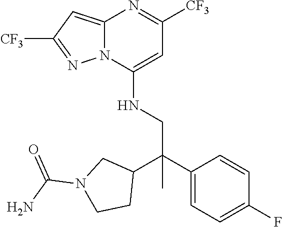 [SMILES: CC(CNc1cc(C(F)(F)F)nc2cc(C(F)(F)F)nn12)(c1ccc(F)cc1)C1CCN(C(N)=O)C1]

### Step 1. tert-Butyl 3-(1-cyano-1-(4-fluorophenyl)ethyl)pyrrolidine-1-carboxylate

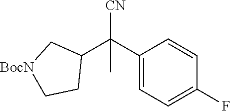 [SMILES: CC(C)(C)OC(=O)N1CCC(C(C)(C#N)c2ccc(F)cc2)C1]

To a solution of 2-(4-fluorophenyl)acetonitrile (350 mg, 2.59 mmol) in THE (5 mL) under nitrogen, NaHMDS (2.59 mL, 2.59 mmol, 1.0 M in THF) was added all at once. The solution was stirred at r.t. for 20 min., and then tert-butyl 3-iodopyrrolidine-1-carboxylate (770 mg, 2.59 mmol) was added over 10 min. The solution was stirred for another 2 h, and then NaHMDS (2.59 mL, 2.59 mmol, 1.0 M in THF) was added all at once. After stirring for 20 min., methyl iodide (0.162 mL, 2.59 mmol) was added, and the solution was stirred at r.t. overnight. The mixture was then diluted with EtOAc and washed with water and brine. The organic phase was collected, dried (Na₂SO₄), filtered, concentrated, and purified by FCC (BIOTAGE® ISOLERA™, pure hexanes to 20% EtOAc) to give the sub-title compound as a yellow oil. LCMS calc. for C₁₄H₁₆FN₂O₂(M+H-t-Bu)⁺: m/z=263.1; found: 263.1.

### Step 2. tert-Butyl 3-(1-amino-2-(4-fluorophenyl)propan-2-yl)pyrrolidine-1-carboxylate

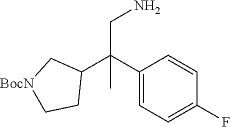 [SMILES: CC(C)(C)OC(=O)N1CCC(C(C)(CN)c2ccc(F)cc2)C1]

To a stirred solution of tert-butyl 3-(1-cyano-1-(4-fluorophenyl)ethyl)pyrrolidine-1-carboxylate (200 mg, 0.628 mmol) and nickel(II) chloride hexahydrate (149 mg, 0.628 mmol) in MeOH (10 mL), NaBH₄ (95 mg, 2.51 mmol) was added portionwise over 15 min. The solution was stirred at r.t. for 2 h and then the mixture was diluted with 200 mL of water and extracted with EtOAc (2×100 mL). The combined organic phases were dried over (Na₂SO₄), filtered, concentrated, and purified by FCC (BIOTAGE® ISOLERA™, pure DCM to 10% MeOH) to give the sub-title compound as a colorless oil. LCMS calc. for C₁₈H₂₈FN₂O₂(M+H)⁺: m/z=323.2; found: 323.2.

### Step 3. N-(2-(4-Fluorophenyl)-2-(pyrrolidin-3-yl)propyl)-2,5-bis(trifluoromethyl)pyrazolo[1,5-a]pyrimidin-7-amine

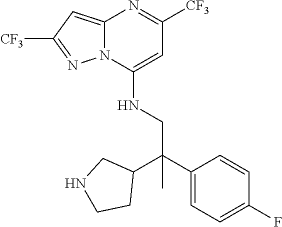 [SMILES: CC(CNc1cc(C(F)(F)F)nc2cc(C(F)(F)F)nn12)(c1ccc(F)cc1)C1CCNC1]

A solution of 7-chloro-2,5-bis(trifluoromethyl)pyrazolo[1,5-a]pyrimidine (45 mg, 0.155 mmol), tert-butyl 3-(1-amino-2-(4-fluorophenyl)propan-2-yl)pyrrolidine-1-carboxylate (50 mg, 0.155 mmol) and DIPEA (0.135 mL, 0.775 mmol) in DMSO (0.5 mL) was heated at 110° C. for 2 h. After this time, the mixture was allowed to cool to r.t., diluted with EtOAc, and washed with water and brine. The organic phase was dried (Na₂SO₄), filtered, and concentrated to give a brown oil. The crude oil was taken up in DCM (0.5 mL) and TFA (0.5 mL) and stirred at r.t. for 1 h. After this time, the mixture was concentrated, diluted with EtOAc, and washed with sat. aq. NaHCO₃ and water. The organic phase was collected, dried (Na₂SO₄), filtered, and concentrated to give a brown oil which was used directly in the next step. LCMS calc. for C₂₁H₂₁F₇N₅ (M+H)⁺: m/z=476.2; found: 476.2.

### Step 4. 3-(1-((2,5-Bis(trifluoromethyl)pyrazolo[1,5-a]pyrimidin-7-yl)amino)-2-(4-fluorophenyl)propan-2-yl)pyrrolidine-1-carboxamide

A stirred solution of N-(2-(4-fluorophenyl)-2-(pyrrolidin-3-yl)propyl)-2,5-bis(trifluoromethyl)pyrazolo[1,5-a]pyrimidin-7-amine (25 mg, 0.053 mmol), phenyl carbamate (7.2 mg, 0.053 mmol) and DIPEA (0.028 mL, 0.158 mmol) in MeCN (0.5 mL) was heated at 65° C. for 2 h. After this time, the mixture was diluted with MeCN and purified by prep.-LCMS (Waters SUNFIRE® C₁₈ column, eluting with a gradient of MeCN/water containing 0.1% TFA, at a flow rate of 60 mL/min., gradient of 39.6-57.6% MeCN in 12 min.). The two diastereomers could be separated.

Example 3 (Peak 1—eluted at 9.6 min.): LCMS calc. for C₂₂H₂₂F₇N₆O (M+H)⁺: m/z=519.2; found: 519.2. ¹H NMR (500 MHz, DMSO) δ 8.35 (t, J=6.8 Hz, 1H), 7.51 (dd, J=8.7, 5.3 Hz, 2H), 7.13 (s, 1H), 7.03 (t, J=8.7 Hz, 2H), 6.23 (s, 1H), 3.90 (dd, J=14.1, 6.8 Hz, 1H), 3.74 (dd, J=14.1, 6.8 Hz, 1H), 3.42 (t, J=9.1 Hz, 1H), 3.19 (td, J=10.0, 6.6 Hz, 1H), 3.04-2.90 (m, 2H), 2.65 (t, J=9.6 Hz, 1H), 2.12 (dt, J=12.5, 6.3 Hz, 1H), 1.84-1.72 (m, 1H), 1.40 (s, 3H).

Example 4 (Peak 2—eluted at 10.2 min.): LCMS calc. for C₂₂H₂₂F₇N₆O (M+H)⁺: m/z=519.2; found: 519.2. ¹H NMR (500 MHz, DMSO) δ 8.32 (t, J=6.8 Hz, 1H), 7.51 (dd, J=8.7, 5.3 Hz, 2H), 7.13 (s, 1H), 7.04 (t, J=8.7 Hz, 2H), 6.32 (s, 1H), 3.92 (dd, J=14.1, 6.7 Hz, 1H), 3.70 (dd, J=14.1, 6.8 Hz, 1H), 3.61 (dd, J=10.2, 7.7 Hz, 1H), 3.31-3.23 (m, 1H), 3.15-3.05 (m, 2H), 2.93-2.82 (m, 1H), 1.65-1.57 (m, 1H), 1.47-1.40 (m, 1H), 1.40 (s, 3H).

The compounds obtained are understood to be diastereoisomers of the title compound (R*)-3-((R*)-1-((2,5-bis(trifluoromethyl)pyrazolo[1,5-a]pyrimidin-7-yl)amino)-2-(4-fluorophenyl)propan-2-yl)pyrrolidine-1-carboxamide and (R*)-3-((S*)-1-((2,5-bis(trifluoromethyl)pyrazolo[1,5-a]pyrimidin-7-yl)amino)-2-(4-fluorophenyl)propan-2-yl)pyrrolidine-1-carboxamide. Each compound was obtained as a racemic mixture and each is separable into its enantiomers (R)-3-((R)-1-((2,5-bis(trifluoromethyl)pyrazolo[1,5-a]pyrimidin-7-yl)amino)-2-(4-fluorophenyl)propan-2-yl)pyrrolidine-1-carboxamide and (S)-3-((S)-1-((2,5-bis(trifluoromethyl)pyrazolo[1,5-a]pyrimidin-7-yl)amino)-2-(4-fluorophenyl)propan-2-yl)pyrrolidine-1-carboxamide, and (R)-3-((S)-1-((2,5-bis(trifluoromethyl)pyrazolo[1,5-a]pyrimidin-7-yl)amino)-2-(4-fluorophenyl)propan-2-yl)pyrrolidine-1-carboxamide and (S)-3-((R)-1-((2,5-bis(trifluoromethyl)pyrazolo[1,5-a]pyrimidin-7-yl)amino)-2-(4-fluorophenyl)propan-2-yl)pyrrolidine-1-carboxamide, respectively.

### Example 5 and 6. 3-(1-((2,5-Bis(trifluoromethyl)pyrazolo[1,5-a]pyrimidin-7-yl)amino)-2-(4-fluorophenyl)propan-2-yl)azetidine-1-carboxamide

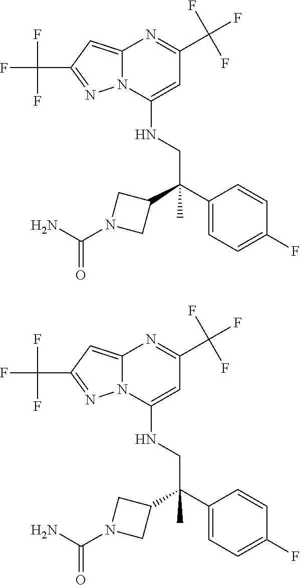 [SMILES: C[C@](CNc1cc(C(F)(F)F)nc2cc(C(F)(F)F)nn12)(c1ccc(F)cc1)C1CN(C(N)=O)C1 ; C[C@@](CNc1cc(C(F)(F)F)nc2cc(C(F)(F)F)nn12)(c1ccc(F)cc1)C1CN(C(N)=O)C1]

N-(2-(Azetidin-3-yl)-2-(4-fluorophenyl)propyl)-2,5-bis(trifluoromethyl)pyrazolo[1,5-a]pyrimidin-7-amine (10 mg, 0.022 mmol) prepared in Example 13, phenyl carbamate (6 mg, 0.043 mmol) and DIPEA (11 μL, 0.065 mmol) in 1,4-dioxane (2 mL) were stirred at 100° C. for 0.5 h. Upon completion, the reaction mixture was diluted with MeOH and purified by prep.-LCMS (XBRIDGE® C₁₈ column, eluting with a gradient of MeCN/water containing 0.1% TFA, at a flow rate of 60 mL/min) to afford title compound as a racemic mixture. Then the mixture was separated by chiral SFC (10% EtOH in hexane, 20 mL/min., R.T. 23.7 min. and 27.1 min., Chiral Tech IBN-5, 5 μm 21×250 mm, Column S/N 884WL001-EA241) to afford two pure enantiomers.

Example 5. Peak 1 (R.T. 23.7 min.). LCMS calc. for C₂₁H₂₀F₇N₆O (M+H)⁺: m/z=505.2; found: 505.2.

Example 6. Peak 2 (R.T. 27.1 min.). LCMS calc. for C₂₁H₂₀F₇N₆O (M+H)⁺: m/z=505.2; found: 505.2.

The compounds obtained are understood to be enantiomers of the title compound (R)-3-(1-((2,5-bis(trifluoromethyl)pyrazolo[1,5-a]pyrimidin-7-yl)amino)-2-(4-fluorophenyl)propan-2-yl)azetidine-1-carboxamide and (S)-3-(1-((2,5-bis(trifluoromethyl)pyrazolo[1,5-a]pyrimidin-7-yl)amino)-2-(4-fluorophenyl)propan-2-yl)azetidine-1-carboxamide.

### Example 7. 3-(1-((5-(Difluoromethyl)-2-(trifluoromethyl)pyrazolo[1,5-a]pyrimidin-7-yl)amino)-2-phenylpropan-2-yl)azetidine-1-sulfonamide

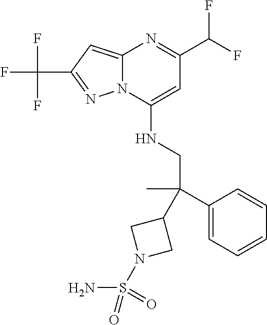 [SMILES: CC(CNc1cc(C(F)F)nc2cc(C(F)(F)F)nn12)(c1ccccc1)C1CN(S(N)(=O)=O)C1]

### Step 1. 5-(Difluoromethyl)-2-(trifluoromethyl)pyrazolo[1,5-a]pyrimidin-7-ol

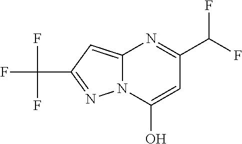 [SMILES: Oc1cc(C(F)F)nc2cc(C(F)(F)F)nn12]

A mixture of 5 3-(trifluoromethyl)-1H-pyrazol-5-amine (2.0 g, 13.24 mmol), ethyl (Z)-4,4-difluoro-3-hydroxybut-2-enoate (2.2 g, 13.24 mmol) and TFA (1.02 mL, 12.2 mmol) in 15 mL DMSO was stirred at r.t. overnight. The resulting mixture was poured into water while stirring at 0° C. to afford the sub-title compound as a white solid which was filtered and dried. LCMS calc. for C₈H₅F₅N₃O (M+H)⁺: m/z=254; found: 254.

### Step 2. 7-Chloro-5-(difluoromethyl)-2-(trifluoromethyl)pyrazolo[1,5-a]pyrimidine

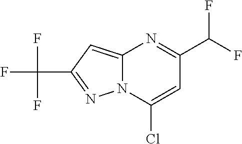 [SMILES: FC(F)c1cc(Cl)n2nc(C(F)(F)F)cc2n1]

A mixture of 5-(difluoromethyl)-2-(trifluoromethyl)pyrazolo[1,5-a]pyrimidin-7-ol (1.5 g, 5.93 mmol), POCl₃ (5.52 mL, 59.3 mmol) and DIPEA (1.03 mL, 5.93 mmol) in toluene (10 mL) was stirred at 100° C. for 3 h. After the full conversion, the mixture was allowed to cool to r.t., and toluene was removed in vacuo. The resulting solution was carefully poured into ice while stirring to afford the sub-title compound as a white solid which was filtered and washed with water and cold hexane, then dried. LCMS calc. for C₈H₄ClF₅N₃(M+H)⁺: m/z=272; found: 272.

### Step 3. N-(2-(Azetidin-3-yl)-2-phenylpropyl)-5-(difluoromethyl)-2-(trifluoromethyl)pyrazolo[1,5-a]pyrimidin-7-amine

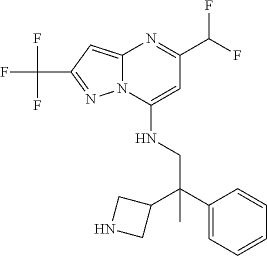 [SMILES: CC(CNc1cc(C(F)F)nc2cc(C(F)(F)F)nn12)(c1ccccc1)C1CNC1]

A mixture of 7-chloro-5-(difluoromethyl)-2-(trifluoromethyl)pyrazolo[1,5-a]pyrimidine (50 mg, 0.184 mmol), tert-butyl 3-(1-amino-2-phenylpropan-2-yl)azetidine-1-carboxylate (53.5 mg, 0.184 mmol) and DIPEA (0.096 mL, 0.552 mmol) in 1,4-dioxane (5 mL) was stirred at 100° C. for 1 h. After complete conversion, the mixture was allowed to cool to r.t., and TFA 2 ml was added and stirred for 1 h at r.t. Then the reaction mixture was diluted with MeOH and purified by prep. HPLC to afford the sub-title compound. LCMS calc. for C₂₀H₂₁F₅N₅ (M+H)⁺: m/z=426.2; found: 426.2.

### Step 4. 3-(1-((5-(Difluoromethyl)-2-(trifluoromethyl)pyrazolo[1,5-a]pyrimidin-7-yl)amino)-2-phenylpropan-2-yl)azetidine-1-sulfonamide

A mixture of N-(2-(azetidin-3-yl)-2-phenylpropyl)-5-(difluoromethyl)-2-(trifluoromethyl)pyrazolo[1,5-a]pyrimidin-7-amine (10 mg, 0.024 mmol), phenyl sulfamate (12 mg, 0.071 mmol) and DIPEA (0.021 mL, 0.118 mmol) in 1,4-dioxane (1 mL) was stirred at 100° C. for 2 h. The reaction mixture was allowed to cool to r.t. and diluted with MeOH and purified by prep. HPLC to afford the title compound. LCMS calc. for C₂₀H₂₂F₅N₆O₂S (M+H)⁺: m/z=505.1; found: 505.1.

The title compound was prepared as a racemic mixture. The racemic mixture is separable into its enantiomers (R)-3-(1-((5-(difluoromethyl)-2-(trifluoromethyl)pyrazolo[1,5-a]pyrimidin-7-yl)amino)-2-phenylpropan-2-yl)azetidine-1-sulfonamide and (S)-3-(1-((5-(difluoromethyl)-2-(trifluoromethyl)pyrazolo[1,5-a]pyrimidin-7-yl)amino)-2-phenylpropan-2-yl)azetidine-1-sulfonamide.

### Example 8. 3-(1-((5-(Difluoromethyl)-2-(trifluoromethyl)pyrazolo[1,5-a]pyrimidin-7-yl)amino)-2-phenylpropan-2-yl)azetidine-1-carboxamide

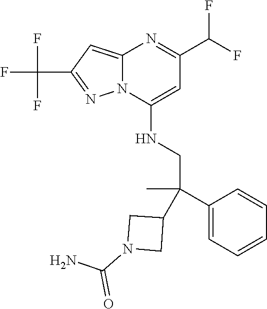 [SMILES: CC(CNc1cc(C(F)F)nc2cc(C(F)(F)F)nn12)(c1ccccc1)C1CN(C(N)=O)C1]

A mixture of N-(2-(azetidin-3-yl)-2-phenylpropyl)-5-(difluoromethyl)-2-(trifluoromethyl)pyrazolo[1,5-a]pyrimidin-7-amine (10 mg, 0.024 mmol), phenyl carbamate (6.5 mg, 0.047 mmol) and DIPEA (0.021 mL, 0.118 mmol) in 1,4-dioxane (1 mL) was stirred at 100° C. for 1 h. The reaction mixture was allowed to cool to r.t., diluted with MeOH and purified by prep. HPLC to afford the title compound. LCMS calc. for C₂₁H₂₂F₅N₆O (M+H)⁺: m/z=469.2; found: 469.2.

The title compound was prepared as a racemic mixture. The racemic mixture is separable into its enantiomers (R)-3-(1-((5-(difluoromethyl)-2-(trifluoromethyl)pyrazolo[1,5-a]pyrimidin-7-yl)amino)-2-phenylpropan-2-yl)azetidine-1-carboxamide and (S)-3-(1-((5-(difluoromethyl)-2-(trifluoromethyl)pyrazolo[1,5-a]pyrimidin-7-yl)amino)-2-phenylpropan-2-yl)azetidine-1-carboxamide.

### Example 9. N-(2-(1-(5-Amino-1H-1,2,4-triazol-3-yl)azetidin-3-yl)-2-phenylpropyl)-2,5-bis(trifluoromethyl)pyrazolo[1,5-a]pyrimidin-7-amine

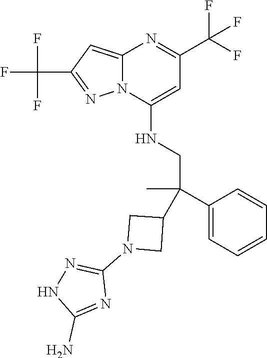 [SMILES: CC(CNc1cc(C(F)(F)F)nc2cc(C(F)(F)F)nn12)(c1ccccc1)C1CN(c2n[nH]c(N)n2)C1]

N-(2-(azetidin-3-yl)-2-(4-fluorophenyl)propyl)-2,5-bis(trifluoromethyl)pyrazolo[1,5-a]pyrimidin-7-amine (10 mg, 0.022 mmol), diphenyl cyanocarbonimidate (6.5 mg, 0.027 mmol) and DIPEA (11 μL, 0.065 mmol) in 1,4-dioxane (2 mL) were stirred at 100° C. for 0.5 h. Then, the reaction mixture was allowed to cool to r.t. and 1M solution of hydrazine (0.068 mL, 0.068 mmol) in EtOH was added. The reaction mixture was stirred at 60° C. for 30 min. Upon completion, the reaction mixture was diluted with MeOH and purified by prep.-LCMS (XBRIDGE® C₁₈ column, eluting with a gradient of MeCN/water containing 0.1% TFA, at a flow rate of 60 mL/min) to afford title compound. LCMS calc. for C₂₂H₂₂F₆N₉ (M+H)⁺: m/z=526.2; found: 526.2.

The title compound was prepared as a racemic mixture. The racemic mixture is separable into its enantiomers (R)—N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine and (S)—N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine.

### Example 10. 3-(1-((2,5-Bis(trifluoromethyl)pyrazolo[1,5-a]pyrimidin-7-yl)amino)-2-phenylpropan-2-yl)azetidine-1-carboxamide

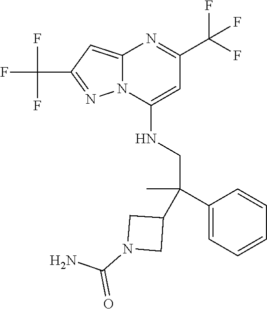 [SMILES: CC(CNc1cc(C(F)(F)F)nc2cc(C(F)(F)F)nn12)(c1ccccc1)C1CN(C(N)=O)C1]

N-(2-(azetidin-3-yl)-2-phenylpropyl)-2,5-bis(trifluoromethyl)pyrazolo[1,5-a]pyrimidin-7-amine (10 mg, 0.023 mmol), phenyl carbamate (6.2 mg, 0.045 mmol) and DIPEA (0.020 mL, 0.113 mmol) in 1,4-dioxane (2 mL) were stirred at 100° C. for 0.5 h. Upon completion, the reaction mixture was diluted with MeOH and purified by prep.-LCMS (XBRIDGE® C₁₈ column, eluting with a gradient of MeCN/water containing 0.1% TFA, at a flow rate of 60 mL/min) to afford title compound. LCMS calc. for C₂₁H₂₁F₆N₆O (M+H)⁺: m/z=487.2; found: 487.2.

The title compound was prepared as a racemic mixture. The racemic mixture is separable into its enantiomers (R)-3-(1-((2,5-bis(trifluoromethyl)pyrazolo[1,5-a]pyrimidin-7-yl)amino)-2-phenylpropan-2-yl)azetidine-1-carboxamide and (S)-3-(1-((2,5-bis(trifluoromethyl)pyrazolo[1,5-a]pyrimidin-7-yl)amino)-2-phenylpropan-2-yl)azetidine-1-carboxamide.

### Example 11. N-(2-(Azetidin-3-yl)-2-phenylpropyl)-2,5-bis(trifluoromethyl)pyrazolo[1,5-a]pyrimidin-7-amine

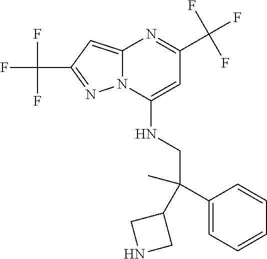 [SMILES: CC(CNc1cc(C(F)(F)F)nc2cc(C(F)(F)F)nn12)(c1ccccc1)C1CNC1]

### Step 1. tert-Butyl 3-(1-amino-2-phenylpropan-2-yl)azetidine-1-carboxylate

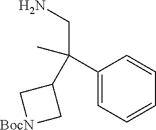 [SMILES: CC(C)(C)OC(=O)N1CC(C(C)(CN)c2ccccc2)C1]

The sub-title compound was prepared following the same procedure as in Example 13 (Step 1, 2) replacing 2-(4-fluorophenyl)propanenitrile with 2-phenylpropanenitrile. LCMS calc. for C₁₇H₂₇N₂O₂ (M+H)⁺: m/z=291.2; found: 291.2.

### Step 2. N-(2-(Azetidin-3-yl)-2-phenylpropyl)-2,5-bis(trifluoromethyl)pyrazolo[1,5-a]pyrimidin-7-amine

7-Chloro-2,5-bis(trifluoromethyl)pyrazolo[1,5-a]pyrimidine (0.1 g, 0.345 mmol), tert-butyl 3-(1-amino-2-phenylpropan-2-yl)azetidine-1-carboxylate (0.100 g, 0.345 mmol) and DIPEA (0.181 mL, 1.036 mmol) in 1,4-dioxane (3 mL) were stirred for 1 h at 100° C. Then the reaction mixture was allowed to cool to r.t. and 3 mL TFA was added. After stirring 15 min. at r.t., the reaction mixture was concentrated, then diluted with 15 mL MeOH. The title compound was obtained as a racemic mixture purified by prep. HPLC purification. LCMS calc. for C₂₀H₂₀F₆N₅ (M+H)⁺: m/z=444.2; found: 444.2.

The title compound was prepared as a racemic mixture. The racemic mixture is separable into its enantiomers (R)—N-(2-(azetidin-3-yl)-2-phenylpropyl)-2,5-bis(trifluoromethyl)pyrazolo[1,5-a]pyrimidin-7-amine and (S)—N-(2-(azetidin-3-yl)-2-phenylpropyl)-2,5-bis(trifluoromethyl)pyrazolo[1,5-a]pyrimidin-7-amine.

### Example 12. 6-(3-(1-((2,5-Bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one

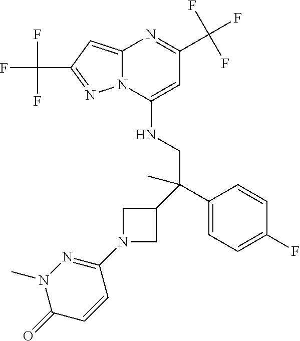 [SMILES: Cn1nc(N2CC(C(C)(CNc3cc(C(F)(F)F)nc4cc(C(F)(F)F)nn34)c3ccc(F)cc3)C2)ccc1=O]

A solution of N-(2-(azetidin-3-yl)-2-(4-fluorophenyl)propyl)-2,5-bis(trifluoromethyl)pyrazolo[1,5-a]pyrimidin-7-amine (10 mg, 0.022 mmol), 6-chloro-2-methylpyridazin-3(2H)-one (9.4 mg, 0.065 mmol) and DIPEA (0.019 mL, 0.108 mmol) in NMP (1 mL) was stirred at 100° C. for 5 h. Then the reaction mixture was allowed to cool to r.t., diluted with MeOH, and purified by prep. HPLC to afford the title compound. LCMS calc. for C₂₅H₂₃F₇N₇O (M+H)⁺: m/z=570.2; found: 570.2.

The title compound was prepared as a racemic mixture. The racemic mixture is separable into its enantiomers (R)-6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one and (S)-6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one.

### Example 13. N-(2-(Azetidin-3-yl)-2-(4-fluorophenyl)propyl)-2,5-bis(trifluoromethyl)pyrazolo[1,5-a]pyrimidin-7-amine

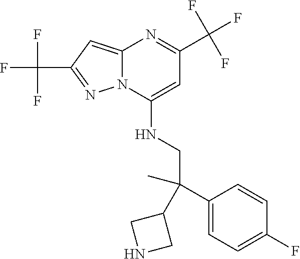 [SMILES: CC(CNc1cc(C(F)(F)F)nc2cc(C(F)(F)F)nn12)(c1ccc(F)cc1)C1CNC1]

### Step 1. tert-Butyl 3-(1-cyano-1-(4-fluorophenyl)ethyl)azetidine-1-carboxylate

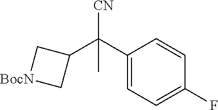 [SMILES: CC(C)(C)OC(=O)N1CC(C(C)(C#N)c2ccc(F)cc2)C1]

A mixture of 2-(4-fluorophenyl)propanenitrile (0.5 g, 3.35 mmol), tert-butyl 3-iodoazetidine-1-carboxylate (1.04 g, 3.69 mmol) and KOtBu (0.451 g, 4.02 mmol) in DMSO (5 mL) was stirred at r.t. overnight. The reaction was quenched with water 15 mL, the product was extracted with EtOAc (20 mL×2). The organic phase was separated, dried over Na₂SO₄, filtered and concentrated. The obtained product was used in the next step without further purification. LCMS calc. for C₁₇H₂₂FN₂O₂(M+H)⁺: m/z=305.2; found: 305.2.

### Step 2. tert-Butyl 3-(1-amino-2-(4-fluorophenyl)propan-2-yl)azetidine-1-carboxylate

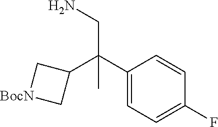 [SMILES: CC(C)(C)OC(=O)N1CC(C(C)(CN)c2ccc(F)cc2)C1]

To a solution of the crude tert-butyl 3-(1-cyano-1-(4-fluorophenyl)ethyl)azetidine-1-carboxylate (1 mmol) and nickel(II) chloride (0.166 g, 1.28 mmol) in MeOH (10 mL) was added NaBH₄ (0.19 g, 4.93 mmol) in several portion at 0° C. The mixture was stirred at 0° C. for 1 h. Upon completion, the reaction was then quenched with saturated NaHCO₃, filtered. The filtrate was concentrated in vacuo and the residue was purified by FCC (15% MeOH/DCM with 5% TEA) to afford the sub-title compound. LCMS calc. for C₁₇H₂₆FN₂O₂(M+H)⁺: m/z=309.2; found: 309.2.

### Step 3. N-(2-(Azetidin-3-yl)-2-(4-fluorophenyl)propyl)-2,5-bis(trifluoromethyl)pyrazolo[1,5-a]pyrimidin-7-amine

7-Chloro-2,5-bis(trifluoromethyl)pyrazolo[1,5-a]pyrimidine (0.1 g, 0.345 mmol), tert-butyl 3-(1-amino-2-(4-fluorophenyl)propan-2-yl)azetidine-1-carboxylate (0.107 g, 0.345 mmol) and DIPEA (0.181 mL, 1.036 mmol) in 1,4-dioxane (3 mL) were stirred for 1 h at 100° C. Then the reaction mixture was allowed to cool to r.t., and 3 mL TFA was added. After stirring for 15 min. at r.t., the reaction mixture was concentrated, then diluted with 15 mL MeOH. The reaction mixture was purified by rep HPLC to afford the title compound. LCMS calc. for C₂₀H₁₉F₇N₅ (M+H)⁺: m/z=462.2; found: 462.2.

The title compound was prepared as a racemic mixture. The racemic mixture is separable into its enantiomers (R)—N-(2-(azetidin-3-yl)-2-(4-fluorophenyl)propyl)-2,5-bis(trifluoromethyl)pyrazolo[1,5-a]pyrimidin-7-amine and (S)—N-(2-(azetidin-3-yl)-2-(4-fluorophenyl)propyl)-2,5-bis(trifluoromethyl)pyrazolo[1,5-a]pyrimidin-7-amine.

### Example 14. 3-(1-((2,5-Bis(trifluoromethyl)pyrazolo[1,5-a]pyrimidin-7-yl)amino)-2-(4-fluorophenyl)propan-2-yl)-N′-cyanoazetidine-1-carboximidamide

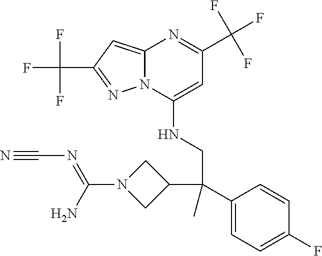 [SMILES: CC(CNc1cc(C(F)(F)F)nc2cc(C(F)(F)F)nn12)(c1ccc(F)cc1)C1CN(/C(N)=N/C#N)C1]

N-(2-(Azetidin-3-yl)-2-(4-fluorophenyl)propyl)-2,5-bis(trifluoromethyl)pyrazolo[1,5-a]pyrimidin-7-amine (10 mg, 0.022 mmol), diphenyl cyanocarbonimidate (10 mg, 0.043 mmol) and DIPEA (11 μL, 0.065 mmol) in 1,4-dioxane (2 mL) were stirred at 100° C. for 0.5 h. After the completion, to the reaction mixture was added ammonium hydroxide (0.5 mL, 30% in water), and the reaction mixture stirred at 100° C. for 0.5 h, then diluted in MeOH and the reaction mixture was purified with prep. HPLC to afford the title compound. LCMS calc. for C₂₂H₂₀F₇N₈ (M+H)⁺: m/z=529.2; found: 529.2.

The title compound was prepared as a racemic mixture. The racemic mixture is separable into its enantiomers (R)-3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide and (S)-3-(1-((2,5-bis(trifluoromethyl)pyrazolo[1,5-a]pyrimidin-7-yl)amino)-2-(4-fluorophenyl)propan-2-yl)-N′-cyanoazetidine-1-carboximidamide.

### Example 15. 4-(2-((2,5-Bis(trifluoromethyl)pyrazolo[1,5-a]pyrimidin-7-yl)amino)-1-(4-fluorophenyl)ethyl)piperidine-1-carboxamide

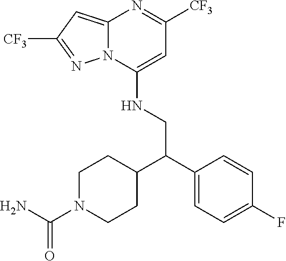 [SMILES: NC(=O)N1CCC(C(CNc2cc(C(F)(F)F)nc3cc(C(F)(F)F)nn23)c2ccc(F)cc2)CC1]

### Step 1. tert-Butyl 4-(2-amino-1-(4-fluorophenyl)ethyl)piperidine-1-carboxylate

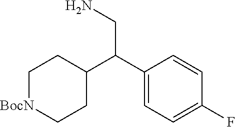 [SMILES: CC(C)(C)OC(=O)N1CCC(C(CN)c2ccc(F)cc2)CC1]

A solution of tert-butyl 4-(cyano(4-fluorophenyl)methyl)piperidine-1-carboxylate (750 mg, 2.4 mmol) (see Example 2, Step 1 for preparation) and BH₃-THF (6.0 mL, 6.0 mmol, 1 M in THF) in THF (5 mL) was heated to 60° C. for 4 h. After this time, the mixture was cooled to r.t., diluted with EtOAc, and then washed with aq. 1 M NaOH and brine. The organic phase was dried over sodium sulfate, filtered, concentrated, and purified by FCC (BIOTAGE® ISOLERA™, pure DCM to 10% MeOH) to give the desired product. LCMS calc. for C₁₄H₂₀FN₂O₂(M+H-t-Bu)⁺: m/z=267.1; found: 267.1.

### Step 2. N-(2-(4-Fluorophenyl)-2-(piperidin-4-yl)ethyl)-2,5-bis(trifluoromethyl)pyrazolo[,5-a]pyrimidin-7-amine

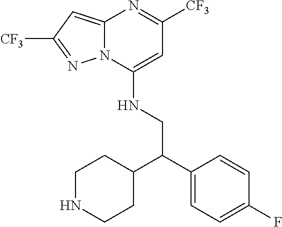 [SMILES: Fc1ccc(C(CNc2cc(C(F)(F)F)nc3cc(C(F)(F)F)nn23)C2CCNCC2)cc1]

The procedure from Example 2, Step 4 was followed using tert-butyl 4-(2-amino-1-(4-fluorophenyl)ethyl)piperidine-1-carboxylate in place of tert-butyl 4-(1-amino-2-(4-fluorophenyl)propan-2-yl)piperidine-1-carboxylate. The crude was used directly in the next step. LCMS calc. for C₂₁H₂₁F₇N₅ (M+H)⁺: m/z=476.2; found: 476.2.

### Step 3. 4-(2-((2,5-Bis(trifluoromethyl)pyrazolo[1,5-a]pyrimidin-7-yl)amino)-1-(4-fluorophenyl)ethyl)piperidine-1-carboxamide

The procedure from Example 2, Step 5 was followed using N-(2-(4-fluorophenyl)-2-(piperidin-4-yl)ethyl)-2,5-bis(trifluoromethyl)pyrazolo[1,5-a]pyrimidin-7-amine in place of N-(2-(4-fluorophenyl)-2-(piperidin-4-yl)propyl)-2,5-bis(trifluoromethyl)pyrazolo[1,5-a]pyrimidin-7-amine. The crude mixture was diluted with MeCN and purified by prep.-LCMS (SUNFIRE® C₁₈ column, eluting with a gradient of MeCN/water containing 0.1% TFA, at flow rate of 60 mL/min) to afford the desired product. LCMS calc. for C₂₂H₂₂F₇N₆O (M+H)⁺: m/z=519.2; found: 519.2.

The title compound was prepared as a racemic mixture. The racemic mixture is separable into its enantiomers (R)-4-(2-((2,5-bis(trifluoromethyl)pyrazolo[1,5-a]pyrimidin-7-yl)amino)-1-(4-fluorophenyl)ethyl)piperidine-1-carboxamide and (S)-4-(2-((2,5-bis(trifluoromethyl)pyrazolo[1,5-a]pyrimidin-7-yl)amino)-1-(4-fluorophenyl)ethyl)piperidine-1-carboxamide.

### Example 16. 3-(1-((2,5-Bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide

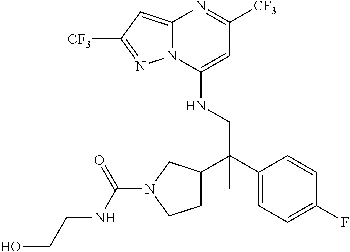 [SMILES: CC(CNc1cc(C(F)(F)F)nc2cc(C(F)(F)F)nn12)(c1ccc(F)cc1)C1CCN(C(=O)NCCO)C1]

A solution of N-(2-(4-fluorophenyl)-2-(pyrrolidin-3-yl)propyl)-2,5-bis(trifluoromethyl)pyrazolo[1,5-a]pyrimidin-7-amine (35 mg, 0.074 mmol) (see Example 3 for preparation), bis(4-nitrophenyl) carbonate (22 mg, 0.074 mmol) and DIPEA (0.026 mL, 0.147 mmol) in MeCN (0.5 mL) was stirred at r.t. for 1 h. After this time, 2-aminoethan-1-ol (4.5 mg, 0.074 mmol) was added and the solution was stirred to 70° C. overnight. The mixture was cooled, diluted with MeCN, and purified by prep.-LCMS (SUNFIRE® C₁₈ column, eluting with a gradient of MeCN/water containing 0.1% TFA, at flow rate of 60 mL/min, gradient of 39.1-57.1% MeCN in 12 min.). The two diastereomers could be separated, with the more potent diastereomer (Peak 1) eluting first (at 9.2 min.). Peak 1 was repurified by prep.-LCMS (XBridge C18 column, eluting with a gradient of MeCN/water containing 0.1% NH₄OH, at flow rate of 60 mL/min). LCMS (Peak 1) calc. for C₂₄H₂₆F₇N₆O₂(M+H)⁺: m/z=563.2; found 563.2.

The title compound can exist as two diastereoisomers that were separated by HPLC although the stereochemistry was not assigned. The diastereomers of the title compound are separable into enantiomers.
Diastereomer A: (R*)-3-((R*)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
Enantiomer A1: (R)-3-((R)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
Enantiomer A2: (S)-3-((S)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
Diastereomer B: (R*)-3-((S*)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
Enantiomer B1: (R)-3-((S)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
Enantiomer B2: (S)-3-((R)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide.

### Example 17. 2-(3-(1-((2,5-Bis(trifluoromethyl)pyrazolo[1,5-a]pyrimidin-7-yl)amino)-2-(4-fluorophenyl)propan-2-yl)pyrrolidin-1-yl)acetamide

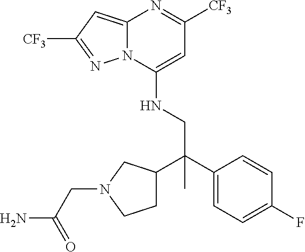 [SMILES: CC(CNc1cc(C(F)(F)F)nc2cc(C(F)(F)F)nn12)(c1ccc(F)cc1)C1CCN(CC(N)=O)C1]

A solution of N-(2-(4-fluorophenyl)-2-(pyrrolidin-3-yl)propyl)-2,5-bis(trifluoromethyl)pyrazolo[1,5-a]pyrimidin-7-amine (28 mg, 0.059 mmol) (see Example 3 for preparation), 2-bromoacetamide (8.9 mg, 0.065 mmol) and DIPEA (0.051 mL, 0.294 mmol) in MeCN (0.5 mL) was heated to 60° C. overnight. The mixture was cooled, diluted with MeCN, and purified by prep.-LCMS (SUNFIRE® C₁₈ column, eluting with a gradient of MeCN/water containing 0.1% TFA, at flow rate of 60 mL/min, gradient of 22.5-40.5% MeCN in 12 min.). The two diastereomers could be separated, with the more potent diastereomer (Peak 1) eluting first (at 11.5 min.). LCMS (Peak 1) calc. for C₂₃H₂₄F₇N₆O (M+H)⁺: m/z=533.2; found 533.2.

The title compound can exist as two diastereoisomers that were separated by HPLC although the stereochemistry was not assigned. The diastereomers of the title compound are separable into enantiomers.
Diastereomer A: 2-((R*)-3-((R*)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
Enantiomer A1: 2-((R)-3-((R)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
Enantiomer A2: 2-((S)-3-((S)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
Diastereomer B: 2-((R*)-3-((S*)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
Enantiomer B1: 2-((R)-3-((S)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
Enantiomer B2: 2-((S)-3-((R)-1-((2,5-bis(trifluoromethyl)pyrazolo[1,5-a]pyrimidin-7-yl)amino)-2-(4-fluorophenyl)propan-2-yl)pyrrolidin-1-yl)acetamide.

### Example 18 and 19. 3-(1-((2,5-Bis(trifluoromethyl)pyrazolo[1,5-a]pyrimidin-7-yl)amino)-2-(4-fluorophenyl)-3-hydroxypropan-2-yl)pyrrolidine-1-carboxamide

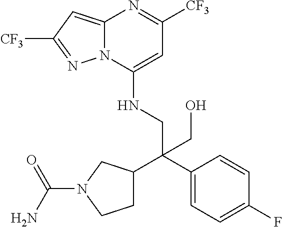 [SMILES: NC(=O)N1CCC(C(CO)(CNc2cc(C(F)(F)F)nc3cc(C(F)(F)F)nn23)c2ccc(F)cc2)C1]

### Step 1. tert-Butyl 3-(cyano(4-fluorophenyl)methyl)pyrrolidine-1-carboxylate

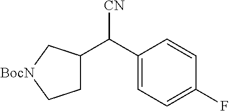 [SMILES: CC(C)(C)OC(=O)N1CCC(C(C#N)c2ccc(F)cc2)C1]

The procedure in Example 3, Step 1 was modified. After the addition of tert-butyl 3-iodopyrrolidine-1-carboxylate and stirring for 2 h, the mixture was quenched. The same workup and purification were used. LCMS calc. for C₁₃H₁₄FN₂O₂(M+H-t-Bu)⁺: m/z=249.1; found: 249.1.

### Step 2. tert-Butyl 3-(1-cyano-1-(4-fluorophenyl)-2-hydroxyethyl)pyrrolidine-1-carboxylate

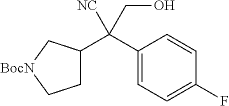 [SMILES: CC(C)(C)OC(=O)N1CCC(C(C#N)(CO)c2ccc(F)cc2)C1]

To a stirred solution of tert-butyl 3-(cyano(4-fluorophenyl)methyl)pyrrolidine-1-carboxylate (400 mg, 1.31 mmol) in THE (5 mL) under nitrogen, NaHMDS (2.6 mL, 2.6 mmol, 1 M in THF) was added all at once. After stirring at r.t. for 15 min., paraformaldehyde (79 mg, 2.63 mmol) was added, and the mixture was stirred for another 1 h. The reaction was then diluted with EtOAc and washed with water and brine. The organic phase was collected, dried over sodium sulfate, filtered, concentrated, and purified by FCC (BIOTAGE® ISOLERA™, pure hexanes to 15% EtOAc) to give the desired product as a yellow oil. LCMS calc. for C₁₄H₁₆FN₂O₃(M+H-t-Bu)⁺: m/z=279.1; found: 279.1.

### Step 3. tert-Butyl 3-(1-amino-2-(4-fluorophenyl)-3-hydroxypropan-2-yl)pyrrolidine-1-carboxylate

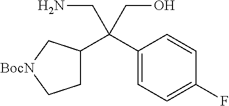 [SMILES: CC(C)(C)OC(=O)N1CCC(C(CN)(CO)c2ccc(F)cc2)C1]

To a stirred solution of tert-butyl 3-(1-cyano-1-(4-fluorophenyl)-2-hydroxyethyl)pyrrolidine-1-carboxylate (150 mg, 0.45 mmol) and nickel(II) chloride hexahydrate (107 mg, 0.45 mmol) in MeOH (20 mL), sodium borohydride (50 mg, 1.35 mmol) was added portionwise over 15 min. After stirring at r.t. for 2 h, the reaction mixture was diluted with aq. ammonium chloride and EtOAc. To the mixture, aq. 1 M NaOH was then added until the emulsion broke. The organic phase was collected, and then the aq. phase was washed with more EtOAc. The combined organic phases were dried over sodium sulfate, filtered, concentrated, and used directly in the next step. LCMS calc. for C₁₈H₂₈FN₂O₃(M+H)⁺: m/z=339.2; found: 339.2.

### Step 4. 3-((2,5-Bis(trifluoromethyl)pyrazolo[1,5-a]pyrimidin-7-yl)amino)-2-(4-fluorophenyl)-2-(pyrrolidin-3-yl)propan-1-ol

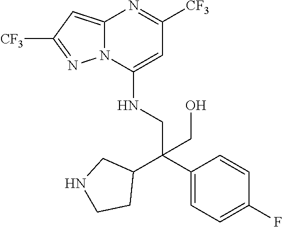 [SMILES: OCC(CNc1cc(C(F)(F)F)nc2cc(C(F)(F)F)nn12)(c1ccc(F)cc1)C1CCNC1]

A solution of 7-chloro-2,5-bis(trifluoromethyl)pyrazolo[1,5-a]pyrimidine (30 mg, 0.104 mmol) (for preparation see Example 1, Step 2), tert-butyl 3-(1-amino-2-(4-fluorophenyl)-3-hydroxypropan-2-yl)pyrrolidine-1-carboxylate (70 mg, 0.21 mmol) and DIPEA (0.018 mL, 0.10 mmol) in DMSO (0.5 mL) was heated to 110° C. overnight. After this time, the mixture was cooled to r.t., diluted with EtOAc, and washed with water and brine. The organic phase was dried over sodium sulfate, filtered, and concentrated to give a brown oil. The crude oil was taken up in DCM (0.5 mL) and TFA (0.5 mL) and stirred at r.t. for 1 h. After this time, the mixture was concentrated, diluted with EtOAc, and washed with aq. ammonium hydroxide and water. The organic phase was collected, dried over sodium sulfate, filtered, and concentrated to give a brown oil which was used directly in the next step. LCMS calc. for C₂₁H₂₁F₇N₅O (M+H)⁺: m/z=492.2; found: 492.2.

### Step 5. 3-(1-((2,5-Bis(trifluoromethyl)pyrazolo[1,5-a]pyrimidin-7-yl)amino)-2-(4-fluorophenyl)-3-hydroxypropan-2-yl)pyrrolidine-1-carboxamide

A stirred solution of 3-((2,5-bis(trifluoromethyl)pyrazolo[1,5-a]pyrimidin-7-yl)amino)-2-(4-fluorophenyl)-2-(pyrrolidin-3-yl)propan-1-ol (80 mg, 0.16 mmol), phenyl carbamate (22 mg, 0.16 mmol) and DIPEA (0.06 mL, 0.33 mmol) in MeCN (0.5 mL) was heated to 65° C. for 2 h. After this time, the mixture was diluted with MeCN and purified by prep.-LCMS (SUNFIRE® C₁₈ column, eluting with a gradient of MeCN/water containing 0.1% TFA, at flow rate of 60 mL/min, gradient of 35.3-53.3% MeCN in 12 min.). The two diastereomers could be separated.

Example 18 (Peak 1—eluted at 9.8 min.): LCMS calc. for C₂₂H₂₂F₇N₆O₂(M+H)⁺: m/z=535.2; found: 535.2.

Example 19 (Peak 2—eluted at 10.1 min.): LCMS calc. for C₂₂H₂₂F₇N₆O₂(M+H)⁺: m/z=535.2; found: 535.2.

The title compound can exist as two diastereoisomers that were separated by HPLC although the stereochemistry was not assigned. The diastereomers of the title compound are separable into enantiomers.
Diastereomer A: (R*)-3-((S*)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
Enantiomer A1: (R)-3-((S)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
Enantiomer A2: (S)-3-((R)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
Diastereomer B: (R*)-3-((R*)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
Enantiomer B1: (R)-3-((R)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide; and
Enantiomer B2: (S)-3-((S)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide.

### Example 20. 3-(2-((2,5-Bis(trifluoromethyl)pyrazolo[1,5-a]pyrimidin-7-yl)amino)-1-(4-fluorophenyl)ethyl)pyrrolidine-1-carboxamide

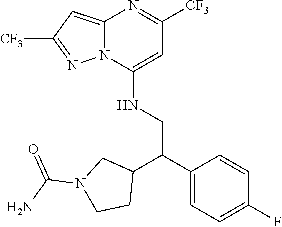 [SMILES: NC(=O)N1CCC(C(CNc2cc(C(F)(F)F)nc3cc(C(F)(F)F)nn23)c2ccc(F)cc2)C1]

### Step 1. tert-Butyl 3-(2-amino-1-(4-fluorophenyl)ethyl)pyrrolidine-1-carboxylate

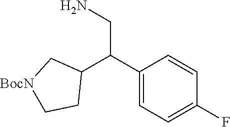 [SMILES: CC(C)(C)OC(=O)N1CCC(C(CN)c2ccc(F)cc2)C1]

To a stirred solution of tert-butyl 3-(cyano(4-fluorophenyl)methyl)pyrrolidine-1-carboxylate (450 mg, 1.48 mmol) (see Example 18, step 1) and nickel(II) chloride hexahydrate (351 mg, 1.48 mmol) in MeOH (20 mL), sodium borohydride (168 mg, 4.44 mmol) was added portionwise over 15 min. After stirring at r.t. for 2 h, the reaction mixture was diluted with aq. ammonium chloride and EtOAc. To the mixture, aq. 1 M NaOH was then added until the emulsion broke. The organic phase was collected, and then the aq. phase was washed with more EtOAc. The combined organic phases were dried over sodium sulfate, filtered, concentrated, and purified by FCC (BIOTAGE® ISOLERA™, pure DCM to 10% MeOH) to give the desired product. LCMS calc. for C₁₃H₁₈FN₂O₂(M+H-t-Bu)⁺: m/z=253.1; found: 253.1.

### Step 2. N-(2-(4-Fluorophenyl)-2-(pyrrolidin-3-yl)ethyl)-2,5-bis(trifluoromethyl)pyrazolo[1,5-a]pyrimidin-7-amine

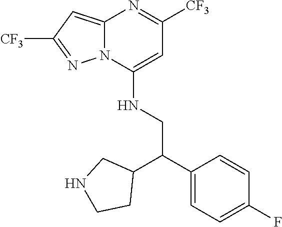 [SMILES: Fc1ccc(C(CNc2cc(C(F)(F)F)nc3cc(C(F)(F)F)nn23)C2CCNC2)cc1]

The procedure in Example 3, Step 3 was followed using tert-butyl 3-(2-amino-1-(4-fluorophenyl)ethyl)pyrrolidine-1-carboxylate in place of tert-butyl 3-(1-amino-2-(4-fluorophenyl)propan-2-yl)pyrrolidine-1-carboxylate. The crude oil was used directly in the next step. LCMS calc. for C₂₀H₁₉F₇N₅ (M+H)⁺: m/z=462.2; found: 462.2.

### Step 3. 3-(2-((2,5-Bis(trifluoromethyl)pyrazolo[1,5-a]pyrimidin-7-yl)amino)-1-(4-fluorophenyl)ethyl)pyrrolidine-1-carboxamide

The procedure in Example 3, Step 4 was followed using N-(2-(4-fluorophenyl)-2-(pyrrolidin-3-yl)ethyl)-2,5-bis(trifluoromethyl)pyrazolo[1,5-a]pyrimidin-7-amine in place of N-(2-(4-fluorophenyl)-2-(pyrrolidin-3-yl)propyl)-2,5-bis(trifluoromethyl)pyrazolo[1,5-a]pyrimidin-7-amine. The crude mixture was diluted with MeCN and purified by prep.-LCMS (SUNFIRE® C₁₈ column, eluting with a gradient of MeCN/water containing 0.1% TFA, at flow rate of 60 mL/min, gradient of 38-56% MeCN in 12 min.). The two diastereomers could be separated, with the more potent diastereomer (Peak 1) eluting first (at 9.6 min.). LCMS (Peak 1) calc. for C₂₁H₂₀F₇N₆O (M+H)⁺: m/z=505.2; found: 505.2.

The title compound can exist as two diastereoisomers that were separated by HPLC although the stereochemistry was not assigned. The diastereomers of the title compound are separable into enantiomers.
Diastereomer A: (R*)-3-((R*)-2-((2,5-bis(trifluoromethyl)pyrazolo[1,5-a]pyrimidin-7-yl)amino)-1-(4-fluorophenyl)ethyl)pyrrolidine-1-carboxamide;
Enantiomer A1: (R)-3-((R)-2-((2,5-bis(trifluoromethyl)pyrazolo[1,5-a]pyrimidin-7-yl)amino)-1-(4-fluorophenyl)ethyl)pyrrolidine-1-carboxamide;
Enantiomer A2: (S)-3-((S)-2-((2,5-bis(trifluoromethyl)pyrazolo[1,5-a]pyrimidin-7-yl)amino)-1-(4-fluorophenyl)ethyl)pyrrolidine-1-carboxamide;
Diastereomer B: (R*)-3-((S*)-2-((2,5-bis(trifluoromethyl)pyrazolo[1,5-a]pyrimidin-7-yl)amino)-1-(4-fluorophenyl)ethyl)pyrrolidine-1-carboxamide;
Enantiomer B1: (R)-3-((S)-2-((2,5-bis(trifluoromethyl)pyrazolo[1,5-a]pyrimidin-7-yl)amino)-1-(4-fluorophenyl)ethyl)pyrrolidine-1-carboxamide; and
Enantiomer B2: (S)-3-((R)-2-((2,5-bis(trifluoromethyl)pyrazolo[1,5-a]pyrimidin-7-yl)amino)-1-(4-fluorophenyl)ethyl)pyrrolidine-1-carboxamide.

### Example 21. 3-(1-((2,5-Bis(trifluoromethyl)pyrazolo[1,5-a]pyrimidin-7-yl)amino)-2-(4-fluorophenyl)-3-hydroxypropan-2-yl)azetidine-1-carboxamide

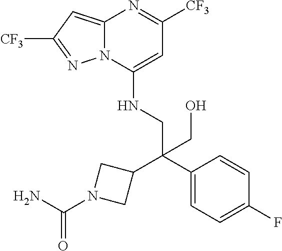 [SMILES: NC(=O)N1CC(C(CO)(CNc2cc(C(F)(F)F)nc3cc(C(F)(F)F)nn23)c2ccc(F)cc2)C1]

### Step 1. tert-Butyl 3-(cyano(4-fluorophenyl)methyl)azetidine-1-carboxylate

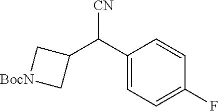 [SMILES: CC(C)(C)OC(=O)N1CC(C(C#N)c2ccc(F)cc2)C1]

To a solution of 2-(4-fluorophenyl)MeCN (2.7 g, 20 mmol) in THE (20 mL) under nitrogen, NaHMDS (20 mL, 20 mmol, 1 M in THF) was added all at once. The solution was stirred at r.t. for 20 min, and then tert-butyl 3-iodoazetidine-1-carboxylate (5.66 g, 20 mmol) was added dropwise in THE (6 mL) over 45 min. The solution was stirred at r.t. overnight, and then was diluted with EtOAc and washed with water and brine. The organic phase was dried over sodium sulfate, filtered, concentrated, and purified by FCC (BIOTAGE® ISOLERA™, pure hexanes to 20% EtOAc). LCMS calc. for C₁₂H₁₂FN₂O₂(M+H-t-Bu)⁺: m/z=235.1; found: 235.1.

### Step 2. tert-Butyl 3-(1-cyano-1-(4-fluorophenyl)-2-hydroxyethyl)azetidine-1-carboxylate

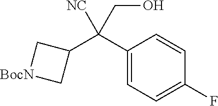 [SMILES: CC(C)(C)OC(=O)N1CC(C(C#N)(CO)c2ccc(F)cc2)C1]

This compound was prepared using the protocol in Example 18, Step 2 using tert-butyl 3-(cyano(4-fluorophenyl)methyl)azetidine-1-carboxylate in place of tert-butyl 3-(cyano(4-fluorophenyl)methyl)pyrrolidine-1-carboxylate. After the workup, the crude was purified by FCC (BIOTAGE® ISOLERA™, pure hexanes to 40% EtOAc) to give the desired product as a yellow oil. LCMS calc. for C₁₃H₁₄FN₂O₃(M+H-t-Bu)⁺: m/z=265.1; found: 265.1.

### Step 3. tert-Butyl 3-(1-amino-2-(4-fluorophenyl)-3-hydroxypropan-2-yl)azetidine-1-carboxylate

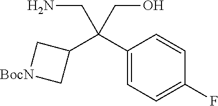 [SMILES: CC(C)(C)OC(=O)N1CC(C(CN)(CO)c2ccc(F)cc2)C1]

This compound was prepared using the protocol in Example 18, Step 3 using tert-butyl 3-(1-cyano-1-(4-fluorophenyl)-2-hydroxyethyl)azetidine-1-carboxylate in place of tert-butyl 3-(1-cyano-1-(4-fluorophenyl)-2-hydroxyethyl)pyrrolidine-1-carboxylate. After the workup, the crude was purified by FCC (BIOTAGE® ISOLERA™, pure DCM to 10% MeOH) to give the desired product as a yellow oil. LCMS calc. for C₁₇H₂₆FN₂O₃(M+H)⁺: m/z=325.2; found: 325.2.

### Step 4. 2-(Azetidin-3-yl)-3-((2,5-bis(trifluoromethyl)pyrazolo[1,5-a]pyrimidin-7-yl)amino)-2-(4-fluorophenyl)propan-1-ol

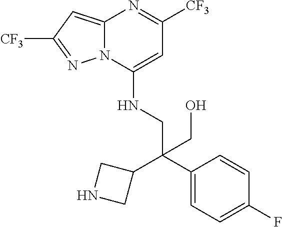 [SMILES: OCC(CNc1cc(C(F)(F)F)nc2cc(C(F)(F)F)nn12)(c1ccc(F)cc1)C1CNC1]

This compound was prepared using the protocol in Example 18, Step 4 using tert-butyl 3-(1-amino-2-(4-fluorophenyl)-3-hydroxypropan-2-yl)azetidine-1-carboxylate in place of tert-butyl 3-(1-amino-2-(4-fluorophenyl)-3-hydroxypropan-2-yl)pyrrolidine-1-carboxylate. After the workup, the crude was used directly in the next step. LCMS calc. for C₂₀H₁₉F₇N₅O (M+H)⁺: m/z=478.1; found: 478.2.

### Step 5. 3-(1-((2,5-Bis(trifluoromethyl)pyrazolo[1,5-a]pyrimidin-7-yl)amino)-2-(4-fluorophenyl)-3-hydroxypropan-2-yl)azetidine-1-carboxamide

This compound was prepared using the protocol in Example 18, Step 5 using 2-(azetidin-3-yl)-3-((2,5-bis(trifluoromethyl)pyrazolo[1,5-a]pyrimidin-7-yl)amino)-2-(4-fluorophenyl)propan-1-ol in place of 3-((2,5-bis(trifluoromethyl)pyrazolo[1,5-a]pyrimidin-7-yl)amino)-2-(4-fluorophenyl)-2-(pyrrolidin-3-yl)propan-1-ol. The mixture was diluted with MeCN and purified by prep.-LCMS (SUNFIRE® C₁₈ column, eluting with a gradient of MeCN/water containing 0.1% TFA, at flow rate of 60 mL/min). LCMS calc. for C₂₁H₂₀F₇N₆O₂ (M+H)⁺: m/z=521.2; found: 521.2.

The title compound was prepared as a racemic mixture. The racemic mixture is separable into its enantiomers (R)-3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide and (S)-3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide.

### Example 22. 2-(2-((2,5-Bis(trifluoromethyl)pyrazolo[1,5-a]pyrimidin-7-yl)amino)-1-(4-fluorophenyl)ethyl)-2-azabicyclo[2.2.1]heptan-5-ol

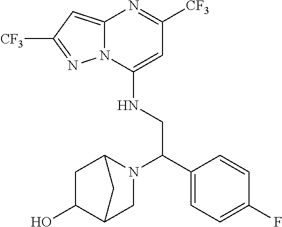 [SMILES: OC1CC2CC1CN2C(CNc1cc(C(F)(F)F)nc2cc(C(F)(F)F)nn12)c1ccc(F)cc1]

### Step 1. 2-((2,5-Bis(trifluoromethyl)pyrazolo[1,5-a]pyrimidin-7-yl)amino)-1-(4-fluorophenyl)ethan-1-ol

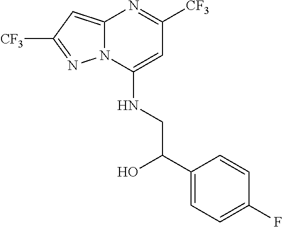 [SMILES: OC(CNc1cc(C(F)(F)F)nc2cc(C(F)(F)F)nn12)c1ccc(F)cc1]

A solution of 7-chloro-2,5-bis(trifluoromethyl)pyrazolo[1,5-a]pyrimidine (1.5 g, 5.18 mmol) (for preparation see Example 1), 2-amino-1-(4-fluorophenyl)ethan-1-ol (0.804 g, 5.18 mmol) and DIPEA (1.8 mL, 10.4 mmol) in MeCN (3 mL) was heated to 55° C. overnight. The next morning, the mixture was cooled to r.t., diluted with EtOAc and washed with water and brine. The organic phase was dried over sodium sulfate, filtered/concentrated, and purified by FCC (BIOTAGE® ISOLERA™, pure hexanes to 30% EtOAc) to give the product as a yellow oil. LCMS calc. for C₁₆H₁₂F₇N₄O (M+H)⁺: m/z=409.1; found: 409.1.

### Step 2. N-(2-Bromo-2-(4-fluorophenyl)ethyl)-2,5-bis(trifluoromethyl)pyrazolo[1,5-a]pyrimidin-7-amine

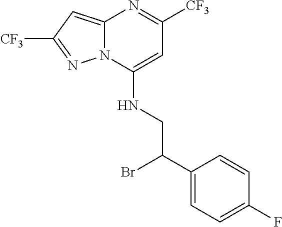 [SMILES: Fc1ccc(C(Br)CNc2cc(C(F)(F)F)nc3cc(C(F)(F)F)nn23)cc1]

To a solution of 2-((2,5-bis(trifluoromethyl)pyrazolo[1,5-a]pyrimidin-7-yl)amino)-1-(4-fluorophenyl)ethan-1-ol (2.1 g, 5.14 mmol) and CBr₄ (1.88 g, 5.66 mmol) in DCM (15 mL) at 0° C., PPh₃ (1.48 g, 5.66 mmol) was added portionwise over 5 min. The solution was stirred to r.t. and stirred for another 1 h. The mixture was concentrated and then purified by FCC (BIOTAGE® ISOLERA™, pure hexanes to 15% EtOAc) to give the product as a white solid. LCMS calc. for C₁₆H₁₁BrF₇N₄(M+H)⁺: m/z=471.0; found: 471.0.

### Step 3. 2-(2-((2,5-Bis(trifluoromethyl)pyrazolo[1,5-a]pyrimidin-7-yl)amino)-1-(4-fluorophenyl)ethyl)-2-azabicyclo[2.2.]]heptan-5-ol

A solution of N-(2-bromo-2-(4-fluorophenyl)ethyl)-2,5-bis(trifluoromethyl)pyrazolo[1,5-a]pyrimidin-7-amine (25 mg, 0.053 mmol), 2-azabicyclo[2.2.1]heptan-5-ol (6 mg, 0.053 mmol) and DIPEA (0.02 mL, 0.106 mmol) in MeCN (0.25 mL) was stirred at 80° C. for 1 h. The mixture was cooled, diluted with MeCN and purified by prep.-LCMS (SunFire C18 column, eluting with a gradient of MeCN/water containing 0.1% TFA, at flow rate of 60 mL/min) to give the desired product as a mixture of diastereomers. LCMS calc. for C₂₂H₂₁F₇N₅O (M+H)⁺: m/z=504.2; found: 504.2.

The title compound can exist as diastereoisomers that can be separated by HPLC. The diastereomers of the title compound are separable into enantiomers.
Diastereomer A: (1R*,4R*)-2-((R*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer A1: (1R,4R)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer A2: (1S,4S)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Diastereomer B: (1R*,4R*)-2-((S*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer B1: (1R,4R)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer B2: (1S,4S)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Diastereomer C: (1S*,4R*)-2-((S*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer C1: (1S,4R)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer C2: (1R,4S)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Diastereomer D: (1S*,4R*)-2-((R*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
Enantiomer D1: (1S,4R)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol; and
Enantiomer D2: (1R,4S)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol.

### Example 23. 2-(2-((2,5-Bis(trifluoromethyl)pyrazolo[1,5-a]pyrimidin-7-yl)amino)-1-(4-fluorophenyl)ethyl)-2-azaspiro[3.5]nonan-7-ol

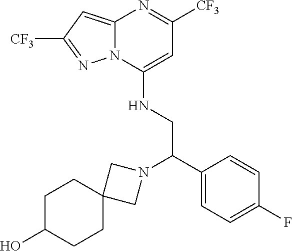 [SMILES: OC1CCC2(CC1)CN(C(CNc1cc(C(F)(F)F)nc3cc(C(F)(F)F)nn13)c1ccc(F)cc1)C2]

The procedure in Example 22, Step 3 was followed using 2-azaspiro[3.5]nonan-7-ol in place of 2-azabicyclo[2.2.1]heptan-5-ol. Purification by prep.-LCMS (SunFire C18 column, eluting with a gradient of MeCN/water containing 0.1% TFA, at flow rate of 60 mL/min). LCMS calc. for C₂₄H₂₅F₇N₅O (M+H)⁺: m/z=532.2; found: 532.2.

The title compound was prepared as a racemic mixture. The racemic mixture is separable into its enantiomers (R)-2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol and (S)-2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol.

### Example 24. N-(2-(4-Fluorophenyl)-2-(3-(pyridin-2-yl)azetidin-1-yl)ethyl)-2,5-bis(trifluoromethyl)pyrazolo[1,5-a]pyrimidin-7-amine

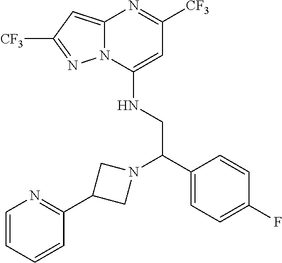 [SMILES: Fc1ccc(C(CNc2cc(C(F)(F)F)nc3cc(C(F)(F)F)nn23)N2CC(c3ccccn3)C2)cc1]

The procedure in Example 22, Step 3 was followed using 2-(azetidin-3-yl)pyridine in place of 2-azabicyclo[2.2.1]heptan-5-ol. Purification by prep.-LCMS (SunFire C18 column, eluting with a gradient of MeCN/water containing 0.1% TFA, at flow rate of 60 mL/min). LCMS calc. for C₂₄H₂₀F₇N₆ (M+H)⁺: m/z=525.2; found: 525.2.

The title compound was prepared as a racemic mixture. The racemic mixture is separable into its enantiomers (R)—N-(2-(4-Fluorophenyl)-2-(3-(pyridin-2-yl)azetidin-1-yl)ethyl)-2,5-bis(trifluoromethyl)pyrazolo[1,5-a]pyrimidin-7-amine and (S)—N-(2-(4-Fluorophenyl)-2-(3-(pyridin-2-yl)azetidin-1-yl)ethyl)-2,5-bis(trifluoromethyl)pyrazolo[1,5-a]pyrimidin-7-amine.

### Example 25. 2-(2-((2,5-Bis(trifluoromethyl)pyrazolo[1,5-a]pyrimidin-7-yl)amino)-1-(4-fluorophenyl)ethyl)-7-oxa-2,5-diazaspiro[3.5]nonan-6-one

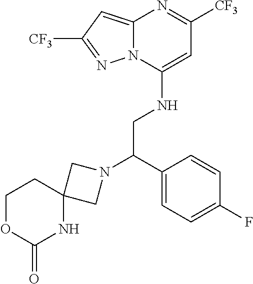 [SMILES: O=C1NC2(CCO1)CN(C(CNc1cc(C(F)(F)F)nc3cc(C(F)(F)F)nn13)c1ccc(F)cc1)C2]

The procedure in Example 22, Step 3 was followed using 7-oxa-2,5-diazaspiro[3.5]nonan-6-one hydrochloride in place of 2-azabicyclo[2.2.1]heptan-5-ol. Purification by prep.-LCMS (SunFire C18 column, eluting with a gradient of MeCN/water containing 0.1% TFA, at flow rate of 60 mL/min) followed by chiral SFC (Cellulose-5 column, 25% MeOH in CO₂) afforded two enantiomers. The enantiomer that eluted first (Peak 1) was the more potent isomer and was repurified by prep.-LCMS (SunFire C18 column, eluting with a gradient of MeCN/water containing 0.1% TFA, at flow rate of 60 mL/min). LCMS (Peak 1) calc. for C₂₂H₂₀F₇N₆O₂(M+H)⁺: m/z=533.2; found: 533.2. NMR of free base: ¹H NMR (400 MHz, DMSO-d₆) δ 8.79 (s, 1H), 7.76 (s, 1H), 7.37-7.27 (m, 2H), 7.14 (s, 1H), 7.08-6.98 (m, 2H), 6.10 (s, 1H), 4.19-4.09 (m, 2H), 3.75-3.66 (m, 1H), 3.64-3.52 (m, 3H), 3.17 (d, J=6.9 Hz, 1H), 3.06 (dd, J=7.1, 2.5 Hz, 1H), 2.78 (d, J=7.1 Hz, 1H), 2.15-2.09 (m, 2H).

The two enantiomers of the title compound are (R)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one and (S)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one, which were not assigned.

### Example 26. 1-(2-((2,5-Bis(trifluoromethyl)pyrazolo[1,5-a]pyrimidin-7-yl)amino)-1-(4-fluorophenyl)ethyl)-N-(methyl-d₃)azetidine-3-carboxamide

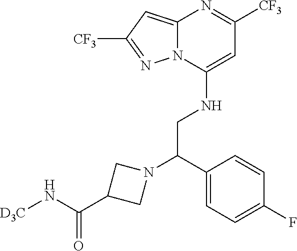 [SMILES: [2H]C([2H])([2H])NC(=O)C1CN(C(CNc2cc(C(F)(F)F)nc3cc(C(F)(F)F)nn23)c2ccc(F)cc2)C1]

### Step 1. 1-(2-((2,5-Bis(trifluoromethyl)pyrazolo[1,5-a]pyrimidin-7-yl)amino)-1-(4-fluorophenyl)ethyl)azetidine-3-carboxylic acid

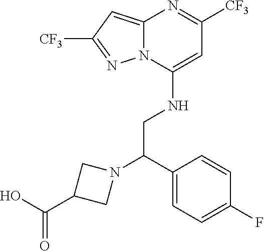 [SMILES: O=C(O)C1CN(C(CNc2cc(C(F)(F)F)nc3cc(C(F)(F)F)nn23)c2ccc(F)cc2)C1]

A solution of N-(2-bromo-2-(4-fluorophenyl)ethyl)-2,5-bis(trifluoromethyl)pyrazolo[1,5-a]pyrimidin-7-amine (500 mg, 1.06 mmol), tert-butyl azetidine-3-carboxylate (167 mg, 1.06 mmol) and DIPEA (0.56 mL, 3.18 mmol) in MeCN (5 mL) was stirred at 80° C. overnight. After this time, the mixture was cooled, concentrated, taken up in DCM (2 mL) and TFA (3 mL) and stirred at r.t. for 2.5 h. The mixture was then concentrated, diluted with EtOAc and washed with water and brine. The organic phase was dried over sodium sulfate, filtered, concentrated, and purified by LCMS (SunFire C18 column, eluting with a gradient of MeCN/water containing 0.1% TFA, at flow rate of 60 mL/min). LCMS calc. for C₂₀H₁₇F₇N₅O₂(M+H)⁺: m/z=492.1; found: 492.1.

### Step 2. 1-(2-((2,5-Bis(trifluoromethyl)pyrazolo[1,5-a]pyrimidin-7-yl)amino)-1-(4-fluorophenyl)ethyl)-N-(methyl-d₃)azetidine-3-carboxamide

To a stirred solution of 1-(2-((2,5-bis(trifluoromethyl)pyrazolo[1,5-a]pyrimidin-7-yl)amino)-1-(4-fluorophenyl)ethyl)azetidine-3-carboxylic acid (420 mg, 0.855 mmol), methan-d3-amine hydrochloride (241 mg, 3.42 mmol), HOBt (157 mg, 1.026 mmol) and DIPEA (1.5 mL, 8.55 mmol) in MeCN (8 mL), EDC (197 mg, 1.026 mmol) was added. The mixture was stirred at r.t. overnight, and then was diluted with EtOAc and washed with water and brine. The organic phase was dried over sodium sulfate, filtered, concentrated, diluted with MeCN and purified by prep.-LCMS (XBridge C18 column, eluting with a gradient of MeCN/water containing 0.1% NH₄OH, at flow rate of 60 mL/min) followed by chiral SFC (Whelk-O1 column, 25% MeOH in CO₂) affording two enantiomers. The enantiomer that eluted first (Peak 1) was the more potent isomer and was repurified by prep.-LCMS (SunFire C18 column, eluting with a gradient of MeCN/water containing 0.1% TFA, at flow rate of 60 mL/min). LCMS (Peak 1) calc. for C₂₁H₁₇D₃F₇N₆O (M+H)⁺: m/z=508.2; found: 508.2. NMR of free base: ¹H NMR (400 MHz, DMSO-d₆) δ 8.75 (s, 1H), 7.72 (s, 1H), 7.37-7.28 (m, 2H), 7.13 (s, 1H), 7.02 (t, J=8.9 Hz, 2H), 6.10 (s, 1H), 3.71 (dd, J=13.2, 3.8 Hz, 1H), 3.65-3.50 (m, 3H), 3.27 (t, J=6.9 Hz, 1H), 3.18-3.05 (m, 2H), 3.02 (t, J=6.3 Hz, 1H).

The two enantiomers of the title compound are (R)-1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide and (S)-1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide, which were not assigned.

### Example 27. N-(2-(4-Fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine

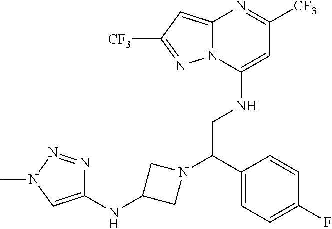 [SMILES: Cn1cc(NC2CN(C(CNc3cc(C(F)(F)F)nc4cc(C(F)(F)F)nn34)c3ccc(F)cc3)C2)nn1]

### Step 1. N-(2-(3-Aminoazetidin-1-yl)-2-(4-fluorophenyl)ethyl)-2,5-bis(trifluoromethyl)pyrazolo[1,5-a]pyrimidin-7-amine

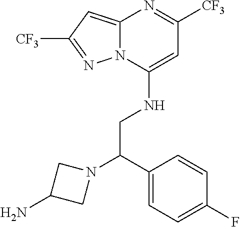 [SMILES: NC1CN(C(CNc2cc(C(F)(F)F)nc3cc(C(F)(F)F)nn23)c2ccc(F)cc2)C1]

A solution of N-(2-bromo-2-(4-fluorophenyl)ethyl)-2,5-bis(trifluoromethyl)pyrazolo[1,5-a]pyrimidin-7-amine (150 mg, 0.318 mmol), tert-butyl azetidin-3-ylcarbamate (55 mg, 0.318 mmol) and DIPEA (0.17 mL, 0.955 mmol) in MeCN (1 mL) was stirred at 80° C. for 1 h. The mixture was cooled to room temperature, concentrated, and then taken up in DCM (0.5 mL) and TFA (0.5 mL) and stirred at r.t. for 30 min. The mixture was then concentrated, diluted with MeCN and purified by prep.-LCMS (SunFire C18 column, eluting with a gradient of MeCN/water containing 0.1% TFA, at flow rate of 60 mL/min). The resulting product was diluted with EtOAc and washed with aq. NaHCO₃. The organic phase was dried over sodium sulfate, concentrated, and then used directly in the next step. LCMS calc. for C₁₉H₁₈F₇N₆ (M+H)⁺: m/z=463.1; found: 463.1.

### Step 2. N-(2-(4-Fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine

A solution of N-(2-(3-aminoazetidin-1-yl)-2-(4-fluorophenyl)ethyl)-2,5-bis(trifluoromethyl)pyrazolo[1,5-a]pyrimidin-7-amine (26 mg, 0.056 mmol), 4-bromo-1-methyl-1H-1,2,3-triazole (9 mg, 0.056 mmol), sodium trimethylsilanolate (0.11 mL, 0.111 mmol, 1 M in THF) and GPhos Pd G6 TES (5.3 mg, 5.6 μmol) in THE (1 mL) was stirred at 85° C. overnight in a sealed vial. The mixture was cooled, concentrated, diluted with MeCN, and purified by prep.-LCMS (SunFire C18 column, eluting with a gradient of MeCN/water containing 0.1% TFA, at flow rate of 60 mL/min). LCMS calc. for C₂₂H₂₁F₇N₉ (M+H)⁺: m/z=544.2; found: 544.2. NMR of free base: ¹H NMR (400 MHz, DMSO-d₆) δ 8.73 (s, 1H), 7.37-7.29 (m, 2H), 7.15 (s, 1H), 7.13 (s, 1H), 7.03 (t, J=8.8 Hz, 2H), 6.10 (s, 1H), 5.71 (d, J=7.8 Hz, 1H), 3.90-3.70 (m, 6H), 3.66-3.55 (m, 2H), 3.38-3.33 (m, 1H), 3.00 (t, J=6.6 Hz, 1H), 2.71 (t, J=6.8 Hz, 1H).

The title compound was prepared as a racemic mixture. The racemic mixture is separable into its enantiomers (R)—N-(2-(4-Fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine and (S)—N-(2-(4-Fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine.

### Example 28. N-(2-(4-Fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine

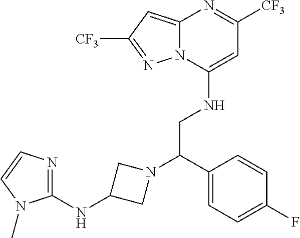 [SMILES: Cn1ccnc1NC1CN(C(CNc2cc(C(F)(F)F)nc3cc(C(F)(F)F)nn23)c2ccc(F)cc2)C1]

The same procedure as Example 27, Step 2 was followed except 2-bromo-1-methyl-1H-imidazole was used in place of 4-bromo-1-methyl-1H-1,2,3-triazole. The crude mixture was concentrated, diluted with MeCN, and purified by prep.-LCMS (SunFire C18 column, eluting with a gradient of MeCN/water containing 0.1% TFA, at flow rate of 60 mL/min). LCMS calc. for C₂₃H₂₂F₇N₈ (M+H)⁺: m/z=543.2; found: 543.2. NMR of free base: ¹H NMR (400 MHz, DMSO-d₆) δ 8.77 (s, 1H), 7.39-7.30 (m, 2H), 7.14 (s, 1H), 7.09-6.98 (m, 2H), 6.58 (d, J=1.5 Hz, 1H), 6.36 (d, J=1.5 Hz, 1H), 6.14 (s, 1H), 5.90 (d, J=7.3 Hz, 1H), 4.19-4.08 (m, 1H), 3.86-3.78 (m, 1H), 3.80-3.72 (m, 1H), 3.68-3.55 (m, 2H), 3.40-3.33 (m, 1H), 3.30 (s, 3H), 3.04 (t, J=6.9 Hz, 1H), 2.80 (t, J=7.0 Hz, 1H).

The title compound was prepared as a racemic mixture. The racemic mixture is separable into its enantiomers (R)—N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine and (S)—N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine.

### Example 29. 6-(2-((2,5-Bis(trifluoromethyl)pyrazolo[1,5-a]pyrimidin-7-yl)amino)-1-(4-fluorophenyl)ethyl)-2,6-diazaspiro[3.3]heptane-2-carboxamide

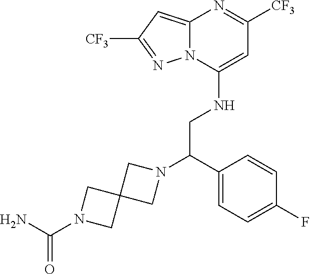 [SMILES: NC(=O)N1CC2(C1)CN(C(CNc1cc(C(F)(F)F)nc3cc(C(F)(F)F)nn13)c1ccc(F)cc1)C2]

### Step 1. tert-Butyl 6-(cyano(4-fluorophenyl)methyl)-2,6-diazaspiro[3.3]heptane-2-carboxylate

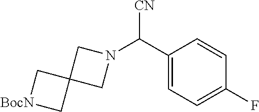 [SMILES: CC(C)(C)OC(=O)N1CC2(C1)CN(C(C#N)c1ccc(F)cc1)C2]

To a stirred solution of 4-fluorobenzaldehyde (2 g, 16.11 mmol) and zinc(II) iodide (0.51 g, 1.61 mmol) in THE (40 mL) was added TMSCN (2.2 mL, 17.73 mmol). The solution was stirred at r.t. for 15 min., and then tert-butyl 2,6-diazaspiro[3.3]heptane-2-carboxylate (3.51 g, 17.73 mmol) was added all at once. The solution was stirred at r.t. for 3 days, and then was diluted with EtOAc and washed with aq. NaHCO₃ and water. The organic phase was collected, dried over sodium sulfate, concentrated, and purified by FCC (BIOTAGE® ISOLERA™, pure hexanes to 30% EtOAc). LCMS calc. for C₁₈H₂₃FN₃O₂(M+H)⁺: m/z=332.2; found: 332.2.

### Step 2. tert-Butyl 6-(2-amino-1-(4-fluorophenyl)ethyl)-2,6-diazaspiro[3.3]heptane-2-carboxylate

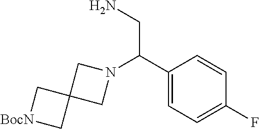 [SMILES: CC(C)(C)OC(=O)N1CC2(C1)CN(C(CN)c1ccc(F)cc1)C2]

To a stirred solution of tert-butyl 6-(cyano(4-fluorophenyl)methyl)-2,6-diazaspiro[3.3]heptane-2-carboxylate (2 g, 6.04 mmol) and nickel(II) chloride hexahydrate (1.72 g, 7.24 mmol) in MeOH (40 mL), sodium borohydride (1.14 g, 30.2 mmol) was added portionwise over 15 min. The solution was stirred at r.t. overnight, and then was diluted with EtOAc and water. The mixture was filtered through celite, and then the organic phase was collected, dried over sodium sulfate, concentrated, and purified by FCC (BIOTAGE® ISOLERA™, pure DCM to 10% MeOH). The mixture was then separated by chiral SFC (Amylose-1 column, 20% MeOH in CO₂) affording two pure enantiomers. The enantiomer that eluted second (Peak 2) was used in the following steps. LCMS calc. for C₁₈H₂₇FN₃O₂(M+H)⁺: m/z=336.2; found: 336.2.

### Step 3. N-(2-(4-Fluorophenyl)-2-(2,6-diazaspiro[3.3]heptan-2-yl)ethyl)-2,5-bis(trifluoromethyl)pyrazolo[1,5-a]pyrimidin-7-amine

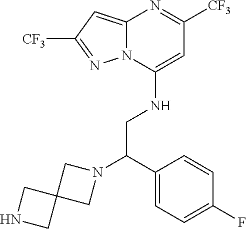 [SMILES: Fc1ccc(C(CNc2cc(C(F)(F)F)nc3cc(C(F)(F)F)nn23)N2CC3(CNC3)C2)cc1]

A solution of 7-chloro-2,5-bis(trifluoromethyl)pyrazolo[1,5-a]pyrimidine (129 mg, 0.447 mmol), tert-butyl 6-(2-amino-1-(4-fluorophenyl)ethyl)-2,6-diazaspiro[3.3]heptane-2-carboxylate (150 mg, 0.447 mmol) and DIPEA (0.23 mL, 1.342 mmol) in DMSO (2.5 mL) was heated to 110° C. for 30 min. The mixture was cooled, diluted with EtOAc and washed with water and brine. The organic phase was dried over sodium sulfate, concentrated, and then taken up in DCM (2 mL) and TFA (2 mL) and stirred at r.t. for 45 min. The solution was then concentrated, diluted with EtOAc and washed with aq. ammonium hydroxide and brine. The organic phase was dried over sodium sulfate, concentrated, and then used directly in the next step. LCMS calc. for C₂₁H₂₀F₇N₆ (M+H)⁺: m/z=489.2; found: 489.2.

### Step 4. 6-(2-((2,5-Bis(trifluoromethyl)pyrazolo[1,5-a]pyrimidin-7-yl)amino)-1-(4-fluorophenyl)ethyl)-2,6-diazaspiro[3.3]heptane-2-carboxamide

A solution of N-(2-(4-fluorophenyl)-2-(2,6-diazaspiro[3.3]heptan-2-yl)ethyl)-2,5-bis(trifluoromethyl)pyrazolo[1,5-a]pyrimidin-7-amine (110 mg, 0.225 mmol), phenyl carbamate (28 mg, 0.203 mmol) and DIPEA (0.12 mL, 0.676 mmol) in MeCN (2 mL) was heated to 60° C. for two h. The mixture was cooled, diluted with MeCN and purified by prep.-LCMS (SunFire C18 column, eluting with a gradient of MeCN/water containing 0.1% TFA, at flow rate of 60 mL/min). LCMS calc. for C₂₂H₂₁F₇N₇O (M+H)⁺: m/z=532.2; found: 532.2.

The title compound was prepared as a racemic mixture. The racemic mixture is separable into its enantiomers (R)-6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide and (S)-6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide.

### Example 30. (3R)-1-(2-((2,5-Bis(trifluoromethyl)pyrazolo[1,5-a]pyrimidin-7-yl)amino)-1-(4-fluorophenyl)ethyl)-3-methylpyrrolidin-3-ol

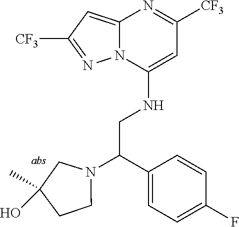 [SMILES: C[C@@]1(O)CCN(C(CNc2cc(C(F)(F)F)nc3cc(C(F)(F)F)nn23)c2ccc(F)cc2)C1]

The procedure in Example 22, Step 3 was followed using (R)-3-methylpyrrolidin-3-ol in place of 2-azabicyclo[2.2.1]heptan-5-ol. Purification by prep.-LCMS (SunFire C18 column, eluting with a gradient of MeCN/water containing 0.1% TFA, at flow rate of 60 mL/min) to give the desired product as a mixture of diastereomers. LCMS calc. for C₂₁H₂₁F₇N₅O (M+H)⁺: m/z=492.2; found: 492.2.

The title compound can exist as two diastereoisomers that can be separated by HPLC.
Diastereomer A: (R)-1-((R)-2-((2,5-bis(trifluoromethyl)pyrazolo[1,5-a]pyrimidin-7-yl)amino)-1-(4-fluorophenyl)ethyl)-3-methylpyrrolidin-3-ol; and
Diastereomer B: (R)-1-((S)-2-((2,5-bis(trifluoromethyl)pyrazolo[1,5-a]pyrimidin-7-yl)amino)-1-(4-fluorophenyl)ethyl)-3-methylpyrrolidin-3-ol.

### Example 31. ((3S)-1-(2-((2,5-Bis(trifluoromethyl)pyrazolo[1,5-a]pyrimidin-7-yl)amino)-1-(4-fluorophenyl)ethyl)pyrrolidin-3-yl)methanol

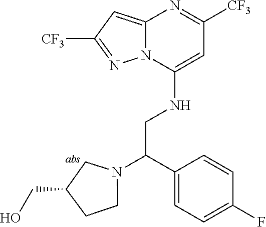 [SMILES: OC[C@H]1CCN(C(CNc2cc(C(F)(F)F)nc3cc(C(F)(F)F)nn23)c2ccc(F)cc2)C1]

The procedure in Example 22, Step 3 was followed using (S)-pyrrolidin-3-ylmethanol in place of 2-azabicyclo[2.2.1]heptan-5-ol. Purification by prep.-LCMS (SunFire C18 column, eluting with a gradient of MeCN/water containing 0.1% TFA, at flow rate of 60 mL/min) to give the desired product as a mixture of diastereomers. LCMS calc. for C₂₁H₂₁F₇N₅O (M+H)⁺: m/z=492.2; found: 492.2.

The title compound can exist as two diastereoisomers that can be separated by HPLC.
Diastereomer A: ((S)-1-((R)-2-((2,5-bis(trifluoromethyl)pyrazolo[1,5-a]pyrimidin-7-yl)amino)-1-(4-fluorophenyl)ethyl)pyrrolidin-3-yl)methanol; and
Diastereomer B: ((S)-1-((S)-2-((2,5-bis(trifluoromethyl)pyrazolo[1,5-a]pyrimidin-7-yl)amino)-1-(4-fluorophenyl)ethyl)pyrrolidin-3-yl)methanol.

### Example 32. 2-(2-((2,5-Bis(trifluoromethyl)pyrazolo[1,5-a]pyrimidin-7-yl)amino)-1-(4-fluorophenyl)ethyl)-2,5-diazaspiro[3.4]octan-6-one

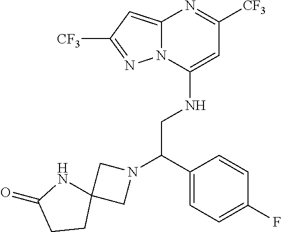 [SMILES: O=C1CCC2(CN(C(CNc3cc(C(F)(F)F)nc4cc(C(F)(F)F)nn34)c3ccc(F)cc3)C2)N1]

The procedure in Example 22, Step 3 was followed using 2,5-diazaspiro[3.4]octan-6-one hydrochloride in place of 2-azabicyclo[2.2.1]heptan-5-ol. Purification by prep.-LCMS (SunFire C18 column, eluting with a gradient of MeCN/water containing 0.1% TFA, at flow rate of 60 mL/min). LCMS calc. for C₂₂H₂₀F₇N₆O (M+H)⁺: m/z=517.2; found: 517.2. NMR of free base: ¹H NMR (400 MHz, DMSO-d₆) δ 8.77 (s, 1H), 8.09 (s, 1H), 7.37-7.29 (m, 2H), 7.14 (s, 1H), 7.03 (t, J=8.8 Hz, 2H), 6.12 (s, 1H), 3.75-3.67 (m, 1H), 3.62-3.52 (m, 3H), 3.21 (d, J=7.0 Hz, 1H), 3.11 (d, J=7.0 Hz, 1H), 2.88 (d, J=7.1 Hz, 1H), 2.29-2.21 (m, 2H), 2.18-2.11 (m, 2H).

The title compound was prepared as a racemic mixture. The racemic mixture is separable into its enantiomers (R)-2-(2-((2,5-Bis(trifluoromethyl)pyrazolo[1,5-a]pyrimidin-7-yl)amino)-1-(4-fluorophenyl)ethyl)-2,5-diazaspiro[3.4]octan-6-one and (S)-2-(2-((2,5-Bis(trifluoromethyl)pyrazolo[1,5-a]pyrimidin-7-yl)amino)-1-(4-fluorophenyl)ethyl)-2,5-diazaspiro[3.4]octan-6-one.

### Example 33. 6-(2-((2,5-Bis(trifluoromethyl)pyrazolo[1,5-a]pyrimidin-7-yl)amino)-1-(4-fluorophenyl)ethyl)-2,6-diazaspiro[3.4]octane-2-carboxamide

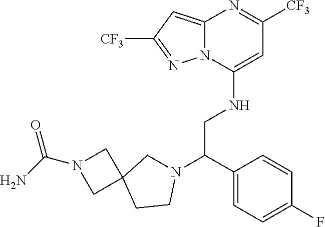 [SMILES: NC(=O)N1CC2(CCN(C(CNc3cc(C(F)(F)F)nc4cc(C(F)(F)F)nn34)c3ccc(F)cc3)C2)C1]

### Step 1. N-(2-(4-Fluorophenyl)-2-(2,6-diazaspiro[3.4]octan-6-yl)ethyl)-2,5-bis(trifluoromethyl)pyrazolo[1,5-a]pyrimidin-7-amine

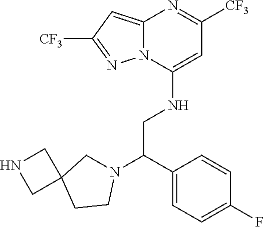 [SMILES: Fc1ccc(C(CNc2cc(C(F)(F)F)nc3cc(C(F)(F)F)nn23)N2CCC3(CNC3)C2)cc1]

A solution of N-(2-bromo-2-(4-fluorophenyl)ethyl)-2,5-bis(trifluoromethyl)pyrazolo[1,5-a]pyrimidin-7-amine (50 mg, 0.106 mmol), tert-butyl 2,6-diazaspiro[3.4]octane-2-carboxylate (22 mg, 0.106 mmol) and DIPEA (0.056 mL, 0.318 mmol) in MeCN (0.5 mL) was stirred at 80° C. for 1 h. After cooling to room temperature, the mixture was concentrated, taken up in DCM (0.5 mL) and TFA (0.5 mL) and stirred at r.t. for 1 h. The mixture was concentrated, diluted with EtOAc and washed with aq. ammonium hydroxide and brine. The organic phase was dried over sodium sulfate, concentrated, and then used directly in the next step. LCMS calc. for C₂₂H₂₂F₇N₆ (M+H)⁺: m/z=503.2; found: 503.2.

### Step 2. 6-(2-((2,5-Bis(trifluoromethyl)pyrazolo[1,5-a]pyrimidin-7-yl)amino)-1-(4-fluorophenyl)ethyl)-2,6-diazaspiro[3.4]octane-2-carboxamide

The crude N-(2-(4-fluorophenyl)-2-(2,6-diazaspiro[3.4]octan-6-yl)ethyl)-2,5-bis(trifluoromethyl)pyrazolo[1,5-a]pyrimidin-7-amine from above was combined with phenyl carbamate (14 mg, 0.106 mmol) and DIPEA (0.056 mL, 0.318 mmol) in MeCN (0.5 mL) and heated to 60° C. for two h. The mixture was cooled, diluted with MeCN and purified by prep.-LCMS (SunFire C18 column, eluting with a gradient of MeCN/water containing 0.1% TFA, at flow rate of 60 mL/min). LCMS calc. for C₂₃H₂₃F₇N₇O (M+H)⁺: m/z=546.2; found: 546.2. NMR of free base: ¹H NMR (400 MHz, DMSO-d₆) δ 8.64 (s, 1H), 7.40-7.30 (m, 2H), 7.13 (s, 1H), 7.04 (t, J=8.8 Hz, 2H), 6.31 (s, 1H), 3.98 (dd, J=13.7, 5.1 Hz, 1H), 3.79 (dd, J=13.7, 7.6 Hz, 1H), 3.73-3.60 (m, 5H), 2.75-2.60 (m, 3H), 2.50-2.45 (m, 1H), 1.98-1.87 (m, 2H).

The title compound was prepared as a racemic mixture. The racemic mixture is separable into its enantiomers (R)-6-(2-((2,5-Bis(trifluoromethyl)pyrazolo[1,5-a]pyrimidin-7-yl)amino)-1-(4-fluorophenyl)ethyl)-2,6-diazaspiro[3.4]octane-2-carboxamide and (S)-6-(2-((2,5-Bis(trifluoromethyl)pyrazolo[1,5-a]pyrimidin-7-yl)amino)-1-(4-fluorophenyl)ethyl)-2,6-diazaspiro[3.4]octane-2-carboxamide.

### Example 34. 2-(2-((2,5-Bis(trifluoromethyl)pyrazolo[1,5-a]pyrimidin-7-yl)amino)-1-(4-fluorophenyl)ethyl)-2-azaspiro[3.3]heptan-5-ol

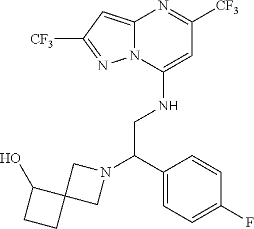 [SMILES: OC1CCC12CN(C(CNc1cc(C(F)(F)F)nc3cc(C(F)(F)F)nn13)c1ccc(F)cc1)C2]

The procedure in Example 22, Step 3 was followed using 2-azaspiro[3.3]heptan-5-ol hydrochloride in place of 2-azabicyclo[2.2.1]heptan-5-ol. Purification by prep.-LCMS (SunFire C18 column, eluting with a gradient of MeCN/water containing 0.1% TFA, at flow rate of 60 mL/min) to give the desired product as a mixture of diastereomers. LCMS calc. for C₂₂H₂₁F₇N₅O (M+H)⁺: m/z=504.2; found: 504.2.

The title compound can exist as two diastereoisomers that can be separated by HPLC. The diastereomers of the title compound are separable into enantiomers.
Diastereomer A: (S*)-2-((R*)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
Enantiomer A1: (S)-2-((R)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
Enantiomer A2: (R)-2-((S)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
Diastereomer B: (S*)-2-((S*)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
Enantiomer B1: (S)-2-((S)-2-((2,5-bis(trifluoromethyl)pyrazolo[1,5-a]pyrimidin-7-yl)amino)-1-(4-fluorophenyl)ethyl)-2-azaspiro[3.3]heptan-5-ol; and
Enantiomer B2: (R)-2-((R)-2-((2,5-bis(trifluoromethyl)pyrazolo[1,5-a]pyrimidin-7-yl)amino)-1-(4-fluorophenyl)ethyl)-2-azaspiro[3.3]heptan-5-ol.

### Example 35. 1-(2-((2,5-Bis(trifluoromethyl)pyrazolo[1,5-a]pyrimidin-7-yl)amino)-1-(4-fluorophenyl)ethyl)-3-methylazetidin-3-ol

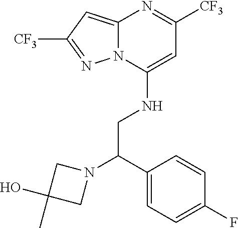 [SMILES: CC1(O)CN(C(CNc2cc(C(F)(F)F)nc3cc(C(F)(F)F)nn23)c2ccc(F)cc2)C1]

The procedure in Example 22, Step 3 was followed using 3-methylazetidin-3-ol hydrochloride in place of 2-azabicyclo[2.2.1]heptan-5-ol. Purification by prep.-LCMS (SunFire C18 column, eluting with a gradient of MeCN/water containing 0.1% TFA, at flow rate of 60 mL/min) followed by chiral LC (Phenomenex cellulose-3 column, 10% EtOH in hexanes) afforded two enantiomers. The enantiomer that eluted second (Peak 2) was the active isomer and was repurified by prep.-LCMS (XBRIDGE® C₁₈ column, eluting with a gradient of MeCN/water containing 0.1% NH₄OH, at flow rate of 60 mL/min). LCMS (Peak 2) calc. for C₂₀H₁₉F₇N₅O (M+H)⁺: m/z=478.1; found: 478.2. NMR of free base: ¹H NMR (500 MHz, DMSO-d₆) δ 8.73 (s, 1H), 7.35-7.29 (m, 2H), 7.13 (s, 1H), 7.03 (t, J=8.8 Hz, 2H), 6.16 (s, 1H), 5.14 (s, 1H), 3.72 (dd, J=12.9, 3.5 Hz, 1H), 3.62-3.50 (m, 2H), 3.38 (dd, J=6.8, 2.3 Hz, 1H), 3.05 (d, J=6.7 Hz, 1H), 2.94 (dd, J=6.9, 2.3 Hz, 1H), 2.73 (d, J=6.8 Hz, 1H), 1.37 (s, 3H).

The two enantiomers of the title compound are (R)-1-(2-((2,5-Bis(trifluoromethyl)pyrazolo[1,5-a]pyrimidin-7-yl)amino)-1-(4-fluorophenyl)ethyl)-3-methylazetidin-3-ol and (S)-1-(2-((2,5-Bis(trifluoromethyl)pyrazolo[1,5-a]pyrimidin-7-yl)amino)-1-(4-fluorophenyl)ethyl)-3-methylazetidin-3-ol, which were not assigned.

### Example 36. 2-(2-((2,5-Bis(trifluoromethyl)pyrazolo[1,5-a]pyrimidin-7-yl)amino)-1-(4-fluorophenyl)ethyl)-2,6-diazaspiro[3.4]octan-7-one

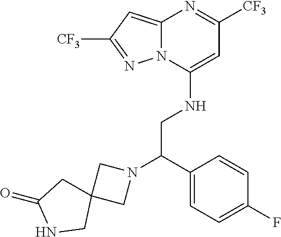 [SMILES: O=C1CC2(CN1)CN(C(CNc1cc(C(F)(F)F)nc3cc(C(F)(F)F)nn13)c1ccc(F)cc1)C2]

The procedure in Example 22, Step 3 was followed using 2,6-diazaspiro[3.4]octan-7-one hydrochloride in place of 2-azabicyclo[2.2.1]heptan-5-ol. Purification by prep.-LCMS (SunFire C18 column, eluting with a gradient of MeCN/water containing 0.1% TFA, at flow rate of 60 mL/min) followed by chiral LC (Phenomenex cellulose-2 column, 20% EtOH in hexanes) afforded two enantiomers. The enantiomer that eluted second (Peak 2) was the active isomer and was repurified by prep.-LCMS (Sunfire C18 column, eluting with a gradient of MeCN/water containing 0.1% TFA, at flow rate of 60 mL/min). LCMS (Peak 2) calc. for C₂₂H₂₀F₇N₆O (M+H)⁺: m/z=517.2; found: 517.2. NMR of free base: ¹H NMR (400 MHz, DMSO-d₆) δ 8.74 (s, 1H), 7.54 (s, 1H), 7.37-7.28 (m, 2H), 7.14 (s, 1H), 7.08-6.98 (m, 2H), 6.14 (s, 1H), 3.71 (dd, J=12.7, 3.2 Hz, 1H), 3.62-3.52 (m, 2H), 3.37 (s, 2H), 3.18 (dd, J=14.9, 6.9 Hz, 2H), 3.10 (t, J=7.5 Hz, 2H), 2.34 (s, 2H).

The two enantiomers of the title compound are (R)-2-(2-((2,5-Bis(trifluoromethyl)pyrazolo[1,5-a]pyrimidin-7-yl)amino)-1-(4-fluorophenyl)ethyl)-2,6-diazaspiro[3.4]octan-7-one and (S)-2-(2-((2,5-Bis(trifluoromethyl)pyrazolo[1,5-a]pyrimidin-7-yl)amino)-1-(4-fluorophenyl)ethyl)-2,6-diazaspiro[3.4]octan-7-one, which were not assigned.

### Example 37. 2-(2-((2,5-Bis(trifluoromethyl)pyrazolo[1,5-a]pyrimidin-7-yl)amino)-1-(4-fluorophenyl)ethyl)-2,5,7-triazaspiro[3.4]octan-6-one

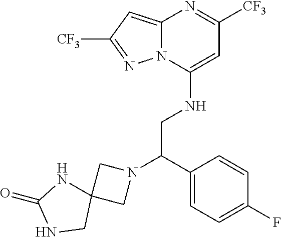 [SMILES: O=C1NCC2(CN(C(CNc3cc(C(F)(F)F)nc4cc(C(F)(F)F)nn34)c3ccc(F)cc3)C2)N1]

The procedure in Example 22, Step 3 was followed using 2,5,7-triazaspiro[3.4]octan-6-one hydrochloride in place of 2-azabicyclo[2.2.1]heptan-5-ol. Purification by prep.-LCMS (XBridge C18 column, eluting with a gradient of MeCN/water containing 0.1% NH₄OH, at flow rate of 60 mL/min) followed by chiral LC (Phenomenex cellulose-2 column, 20% EtOH in Hex) afforded two enantiomers. The enantiomer that eluted second (Peak 2) was the active isomer and was repurified by prep.-LCMS (Sunfire C18 column, eluting with a gradient of MeCN/water containing 0.1% TFA, at flow rate of 60 mL/min). LCMS (Peak 2) calc. for C₂₁H₁₉F₇N₇O (M+H)⁺: m/z=518.2; found: 518.2. NMR of free base: ¹H NMR (400 MHz, DMSO-d₆) δ 8.79-8.73 (m, 1H), 7.36-7.27 (m, 2H), 7.14 (s, 1H), 7.03 (t, J=8.8 Hz, 2H), 6.88 (s, 1H), 6.27 (s, 1H), 6.11 (s, 1H), 3.76-3.66 (m, 1H), 3.64-3.54 (m, 3H), 3.54-3.48 (m, 2H), 3.21 (d, J=7.1 Hz, 1H), 3.12 (dd, J=7.4, 2.2 Hz, 1H), 2.89 (d, J=7.2 Hz, 1H).

The two enantiomers of the title compound are (R)-2-(2-((2,5-Bis(trifluoromethyl)pyrazolo[1,5-a]pyrimidin-7-yl)amino)-1-(4-fluorophenyl)ethyl)-2,5,7-triazaspiro[3.4]octan-6-one and (S)-2-(2-((2,5-Bis(trifluoromethyl)pyrazolo[1,5-a]pyrimidin-7-yl)amino)-1-(4-fluorophenyl)ethyl)-2,5,7-triazaspiro[3.4]octan-6-one, which were not assigned.

### Example 38. 1-(6-(2-((2,5-Bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile

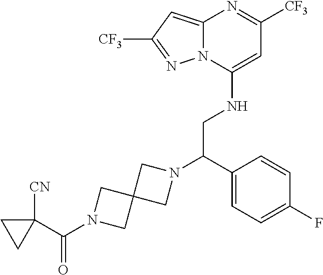 [SMILES: N#CC1(C(=O)N2CC3(C2)CN(C(CNc2cc(C(F)(F)F)nc4cc(C(F)(F)F)nn24)c2ccc(F)cc2)C3)CC1]

To a stirred solution of N-(2-(4-fluorophenyl)-2-(2,6-diazaspiro[3.3]heptan-2-yl)ethyl)-2,5-bis(trifluoromethyl)pyrazolo[1,5-a]pyrimidin-7-amine (20 mg, 0.041 mmol) (chiral material from Example 29, Step 3), 1-cyanocyclopropane-1-carboxylic acid (4.6 mg, 0.041 mmol) and HOBt (6.3 mg, 0.041 mmol) in MeCN (0.5 mL), EDC (7.9 mg, 0.041 mmol) was added. After stirring overnight at r.t., the mixture was diluted with MeCN and purified by prep.-LCMS (SunFire C18 column, eluting with a gradient of MeCN/water containing 0.1% TFA, at flow rate of 60 mL/min). LCMS calc. for C₂₆H₂₃F₇N₇O (M+H)⁺: m/z=582.2; found: 582.2. NMR of free base: ¹H NMR (400 MHz, DMSO-d₆) δ 8.74 (s, 1H), 7.37-7.28 (m, 2H), 7.12 (s, 1H), 7.03 (t, J=8.8 Hz, 2H), 6.12 (s, 1H), 4.57 (s, 2H), 4.01 (s, 2H), 3.73-3.66 (m, 1H), 3.62-3.52 (m, 2H), 3.36-3.21 (m, 4H), 1.57-1.41 (m, 4H).

The title compound was prepared as a racemic mixture. The racemic mixture is separable into its enantiomers (R)-1-(6-(2-((2,5-Bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile and (S)-1-(6-(2-((2,5-Bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile.

### Example 39. 1-(6-(2-((2,5-Bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one

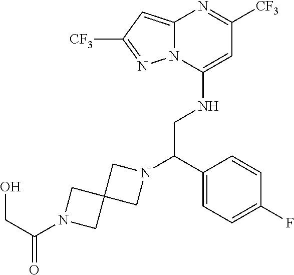 [SMILES: O=C(CO)N1CC2(C1)CN(C(CNc1cc(C(F)(F)F)nc3cc(C(F)(F)F)nn13)c1ccc(F)cc1)C2]

The procedure in Example 38 was following using 2-hydroxyacetic acid in place of 1-cyanocyclopropane-1-carboxylic acid. The crude mixture was diluted with MeCN and purified by prep.-LCMS (SunFire C18 column, eluting with a gradient of MeCN/water containing 0.1% TFA, at flow rate of 60 mL/min). LCMS calc. for C₂₃H₂₂F₇N₆O₂(M+H)⁺: m/z=547.2; found: 547.2. NMR of free base: ¹H NMR (400 MHz, DMSO-d₆) δ 8.73 (s, 1H), 7.36-7.27 (m, 2H), 7.13 (s, 1H), 7.08-6.98 (m, 2H), 6.12 (s, 1H), 4.83 (t, J=6.0 Hz, 1H), 4.26-4.16 (m, 2H), 3.94 (s, 2H), 3.85 (d, J=6.0 Hz, 2H), 3.74-3.65 (m, 1H), 3.60-3.52 (m, 2H), 3.37-3.27 (m, 2H), 3.23 (d, J=7.5 Hz, 2H).

The title compound was prepared as a racemic mixture. The racemic mixture is separable into its enantiomers (R)-1-(6-(2-((2,5-Bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one and (S)-1-(6-(2-((2,5-Bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one.

### Example 40. 6-(2-((2,5-Bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide

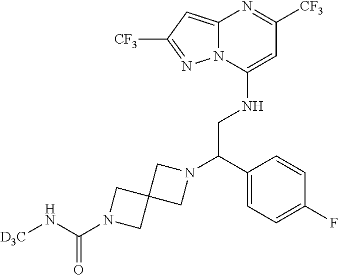 [SMILES: [2H]C([2H])([2H])NC(=O)N1CC2(C1)CN(C(CNc1cc(C(F)(F)F)nc3cc(C(F)(F)F)nn13)c1ccc(F)cc1)C2]

To a solution of methan-d3-amine hydrochloride (2.9 mg, 0.041 mmol) and DIPEA (0.014 mL, 0.082 mmol) in DCM (0.5 mL), triphosgene (4.3 mg, 0.014 mmol) was added and the mixture was stirred for 5 min. at r.t. This solution was then added to a solution of N-(2-(4-fluorophenyl)-2-(2,6-diazaspiro[3.3]heptan-2-yl)ethyl)-2,5-bis(trifluoromethyl)pyrazolo[1,5-a]pyrimidin-7-amine (20 mg, 0.041 mmol) (chiral material from Example 29, Step 3) in DCM (0.5 mL) over around 5 min. The mixture was stirred at r.t. for 1 h, and then was concentrated, diluted with MeCN and purified by prep.-LCMS (SunFire C18 column, eluting with a gradient of MeCN/water containing 0.1% TFA, at flow rate of 60 mL/min). LCMS calc. for C₂₃H₂₀D₃F₇N₇O (M+H)⁺: m/z=549.2; found: 549.2. NMR of free base: ¹H NMR (400 MHz, DMSO-d₆) δ 8.71 (s, 1H), 7.34-7.27 (m, 2H), 7.12 (s, 1H), 7.02 (t, J=8.8 Hz, 2H), 6.14-6.09 (m, 2H), 3.81 (s, 4H), 3.69 (q, J=7.6 Hz, 1H), 3.60-3.51 (m, 2H), 3.29 (d, J=7.4 Hz, 2H), 3.20 (d, J=7.4 Hz, 2H).

The title compound was prepared as a racemic mixture. The racemic mixture is separable into its enantiomers (R)-6-(2-((2,5-Bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide and (S)-6-(2-((2,5-Bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide.

### Example 41. 6-(2-((2,5-Bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide

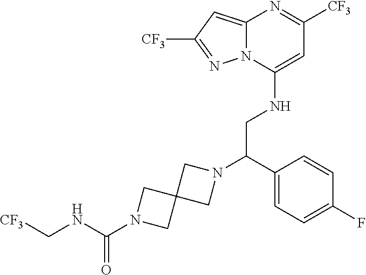 [SMILES: O=C(NCC(F)(F)F)N1CC2(C1)CN(C(CNc1cc(C(F)(F)F)nc3cc(C(F)(F)F)nn13)c1ccc(F)cc1)C2]

To a solution of N-(2-(4-fluorophenyl)-2-(2,6-diazaspiro[3.3]heptan-2-yl)ethyl)-2,5-bis(trifluoromethyl)pyrazolo[1,5-a]pyrimidin-7-amine (20 mg, 0.041 mmol) (chiral material from Example 29, Step 3) and DIPEA (0.014 mL, 0.082 mmol) in MeCN (0.5 mL), 1,1,1-trifluoro-2-isocyanatoethane (5.6 mg, 0.045 mmol) was added and then the solution was stirred at r.t. for 1 h. The mixture was diluted with MeCN and purified by prep.-LCMS (SunFire C18 column, eluting with a gradient of MeCN/water containing 0.1% TFA, at flow rate of 60 mL/min). LCMS calc. for C₂₄H₂₂F₁₀N₇O (M+H)⁺: m/z=614.2; found: 614.2.

The title compound was prepared as a racemic mixture. The racemic mixture is separable into its enantiomers (R)-6-(2-((2,5-Bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide and (S)-6-(2-((2,5-Bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide.

### Example 42. 2-(2-((2,5-Bis(trifluoromethyl)pyrazolo[1,5-a]pyrimidin-7-yl)amino)-1-(4-fluorophenyl)ethyl)-7-oxa-2,5-diazaspiro[3.4]octan-6-one

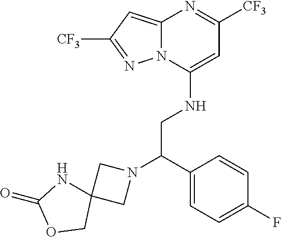 [SMILES: O=C1NC2(CO1)CN(C(CNc1cc(C(F)(F)F)nc3cc(C(F)(F)F)nn13)c1ccc(F)cc1)C2]

The procedure in Example 22, Step 3 was followed using 7-oxa-2,5-diazaspiro[3.4]octan-6-one 2,2,2-trifluoroacetate in place of 2-azabicyclo[2.2.1]heptan-5-ol. Purification by prep.-LCMS (SunFire C18 column, eluting with a gradient of MeCN/water containing 0.1% TFA, at flow rate of 60 mL/min). LCMS calc. for C₂₁H₁₈F₇N₆O₂(M+H)⁺: m/z=519.1; found: 519.1.

The title compound was prepared as a racemic mixture. The racemic mixture is separable into its enantiomers (R)-2-(2-((2,5-Bis(trifluoromethyl)pyrazolo[1,5-a]pyrimidin-7-yl)amino)-1-(4-fluorophenyl)ethyl)-7-oxa-2,5-diazaspiro[3.4]octan-6-one and (S)-2-(2-((2,5-Bis(trifluoromethyl)pyrazolo[1,5-a]pyrimidin-7-yl)amino)-1-(4-fluorophenyl)ethyl)-7-oxa-2,5-diazaspiro[3.4]octan-6-one.

### Example A. MRGPRX2 FLIPR Calcium Mobilization Assay

Chinese hamster ovary (CHO-K1) cells stably expressing human MRGPRX2 were purchased from Genescript (Piscataway, NJ). The cells were maintained in culture medium (Ham's F-12K) containing 10% (v/v) FBS, 200 μg/mL Zeocin, 100 units/mL penicillin G and 100 μg/mL streptomycin (Life Technologies, Carlsbad, CA). For the assay, the cells were harvested and resuspended with culture medium without Zeocin before plating at 8000 cells per well in 20 μL in 384-well black clear bottom cell culture plates (VWR, Radnor, PA). After 24 h culture at 37° C. and 5% CO₂, the cells were loaded with 20 μL/well of calcium dye (FLIPR Calcium 6 Assay Kit, Molecular Devices, San Jose, CA) diluted in loading buffer (1× Hank's Balanced Salt Solution (HBSS), 5 mM probenecid, 20 mM 4-(2-hydroxyethyl)-1-piperazineethanesulfonic acid (HEPES), pH 7.4, VWR, Radnor, PA) followed with 45 min. incubation at 37° C. and 40 min. at r.t. in the dark. Cells were then treated with the addition of μL/well test compounds with a range of increasing concentrations diluted in the assay buffer (1×HBSS, 20 mM HEPES, pH 7.4, VWR, Radnor, PA) with FLIPR Penta (Molecular Devices. San Jose, CA) and incubated for 30 min. at r.t. Ca²⁺ release was measured with FLIPR Penta with 10 s basal fluorescence measurement, then 12.5 μL of 5× agonist Cortistatin-14 (Tocris, Minneapolis, MN) at final concentration corresponding to the EC80 were added followed with continued fluorescence signal monitoring for an additional 110 s. The base line adjusted (median of first 10s base line) max value of the Relative Fluorescence Unit (RFU) was plotted against compound concentrations. Wells with no compound were served as the positive controls, and wells with high concentration of reference antagonist were used as negative controls. IC₅₀ curves were globally fitted with 3- or 4-parameter Hill equation in a Genedata Screener (Genedata Basel, Switzerland).

### Example B: MRGPRX2 IP1 HTRF Assay

This assay measures compound inhibition of myo-Inositol 1 phosphate (IP1) accumulation in CHO-K1 cells. Chinese hamster ovary (CHO-K1) cells stably expressing human MRGPRX2 were purchased from GenScript (Piscataway, NJ).

For the HTRF IP1 determination, an IP-One GqKit (Perkin Elmer Cisbio, Waltham, MA), which includes all reagents and buffers in this protocol, was used. The cells were maintained in culture medium (Ham's F-12K) containing 10% (v/v) FBS, and 200 μg/mL Zeocin. For the assay, the cells were harvested and resuspended in culture medium with 2% FBS and without Zeocin, then were passed through a 40 μm filter. Cells were added at 20000 cells in a 5 μL/well to a 384-well white small volume cell culture plate (Greiner VWR, Radnor, PA) which contained 50 nL/well of test compound serially diluted at a selected concentration range in DMSO. 5 μL/well of prepared 2× Stimulation Buffer2 was then added to plates and was incubated at 37° C. with 5% CO₂ for 1 h. 5 μL/well of agonist Cortistatin 14 (Bio-TechneR&D Systems, Minneapolis, MN) in 1× Stimulation Buffer 2, for a 1 μM final concentration, was then added to plates and incubated at 37° C. with 5% CO₂ for 1 h. 6 μL/well of 1:20 diluted Detection Reagent Mix (d2 and Cryptate) was added to the plates and incubated for 1 h in the dark. Plates were read on the PHERAstar microplate reader (BMG Labtech Cary, NC) to determine the HTRF ratio of the acceptor and donor emission signals. The IP1 concentrations were calculated from a standard curve following the IP-One GQ kit instructions. Wells with DMSO only and wells with high concentration of reference antagonist were used as controls for normalization. Compound IC₅₀ curves were globally fitted with 3- or 4-parameter Hill equation in a Genedata Screener (Genedata Basel, Switzerland).

### Example C: MRGPRX2 β-Arrestin Assay

This assay measures compound inhibition of β-Arrestin recruitment, which is part of the G protein-independent pathway that results from the ligand-activated GPCR phosphorylation by specific GPCR kinases. The PathHunter CHO-K1 MRPGRX2 B-Arrestin Cell line stably expressing ProLink tagged MRGPRX2 and Enzyme Acceptor Tagged B-Arrestin was purchased from Eurofins DiscoverX, (Fremont, CA).

The cells were maintained in culture medium from the Europhins DiscoverX Cell Culture Kit-107 which includes FBS, hygromycin B and G418. For the assay, the cells were harvested and were resuspended with Cell Plating Reagent 2 (Eurofins DiscoverX). Cells were added 10000 cells in 25 μL/well to a 384-well black cell culture plate (Greiner VWR, Radnor, PA) which contained 125 nL/well of compound at a selected serially diluted concentration range or DMSO. The plate was incubated at 37° C. with 5% CO₂ for 1 h. 2 μL/well of agonist Cortistatin 14 was diluted in Protein Dilution Buffer, (Eurofins DiscoverX) for a final concentration of 0.25 μM, was added and the plate was incubated at 37° C. with 5% CO₂ for 90 min. 14 μL/well of Detection Reagent Mix (PathHunter Detection Kit, Eurofins DiscoverX) was added to the plates and further incubated for 1 h in the dark. Plates were read on the PHERAstar microplate reader (BMG Labtech Cary, NC) measuring luminescence 0.1 to 1 s per well. Data was normalized using DMSO only wells and wells with high concentration of reference antagonist as controls. Compound IC₅₀ curves were globally fitted with 3- or 4-parameter Hill equation in a Genedata Screener (Genedata Basel, Switzerland).

### Table of Assay Data for the Assays of Examples A-C

Data obtained for the compounds of the Examples in the assays of Examples A (MRGPRX2 FLIPR), B (“IP1 HTRF”) and C (“MRGPRX2 β-Arrestin”) are shown in Table A. The symbol “++++” indicates an IC₅₀ value of <0.1 nM; “+++” indicates an IC₅₀ value of >0.1 nM but ≤100 nM; the symbol “++” indicates an IC₅₀ value of ≥100 nM but ≤1000 nM; the symbol “+” indicates an IC₅₀ value of >1000 nM. ND indicates no data.

TABLE A

| | MRGPRX2 | MRGPRX2 | MRGPRX2 |
|---|---|---|---|
| Example | FLIPR | IP1 HTRF | β-Arrestin |
| 1 | ND | ND | +++ |
| 2 | ND | ND | +++ |
| 3 | ND | ND | +++ |
| 4 | ND | ND | +++ |
| 5 | ND | +++ | +++ |
| 6 | ND | +++ | +++ |
| 7 | ND | +++ | +++ |
| 8 | ND | +++ | +++ |
| 9 | ND | +++ | +++ |
| 10 | ND | +++ | +++ |
| 11 | ND | +++ | +++ |
| 12 | ND | ND | +++ |
| 13 | +++ | ND | +++ |
| 14 | +++ | +++ | +++ |
| 15 | ND | ND | +++ |
| 16 | ND | +++ | +++ |
| 17 | ND | +++ | +++ |
| 18 | ND | +++ | +++ |
| 19 | ND | ND | +++ |
| 20 | ND | ND | +++ |
| 21 | ND | ND | +++ |
| 22 | ND | ND | +++ |
| 23 | ND | ND | +++ |
| 24 | ND | ND | +++ |
| 25 | ND | ND | +++ |
| 26 | ND | ND | +++ |
| 27 | ND | ND | +++ |
| 28 | ND | ND | +++ |
| 29 | ND | ND | +++ |
| 30 | ND | ND | +++ |
| 31 | ND | ND | +++ |
| 32 | ND | ND | +++ |
| 33 | ND | ND | +++ |
| 34 | ND | ND | +++ |
| 35 | ND | ND | +++ |
| 36 | ND | ND | +++ |
| 37 | ND | ND | +++ |
| 38 | ND | ND | +++ |
| 39 | ND | ND | +++ |
| 40 | ND | ND | +++ |
| 41 | ND | ND | +++ |
| 42 | ND | ND | +++ |

### Example D: Caco2 Assay

Caco-2 cells are grown at 37° C. in an atmosphere of 500 CO₂ in DMEM growth medium supplemented with 1000 (v/v) fetal bovine serum, 1% (v/v) nonessential amino acids, penicillin (100 U/mL), and streptomycin (100 g/mL). Confluent cell monolayers are subcultured every 7 days or 4 days for Caco-2 by treatment with 0.050% trypsin containing 1 M EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values≥300 Ω·cm² are used for transport experiments. To determine the Pₐₚₚ in the absorptive direction (A-B), solution of test compound (50 μM) in HBSS is added to the donor compartment (apical side), while HBSS solution with 4% BSA is added to the receiver compartment (basolateral side). The apical volume was 0.075 mL, and the basolateral volume is 0.25 mL. The incubation period is 120 min. at 37° C. in an atmosphere of 5% CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

$${P}_{a p p} ( cm / s ) = ( F ⋆ VD ) / ( SA ⋆ MD ) ,$$

where the flux rate (F, mass/time) is calculated from the slope of cumulative amounts of compound of interest on the receiver side, SA is the surface area of the cell membrane, VD is the donor volume, and MD is the initial amount of the solution in the donor chamber.

### Example E: Human Whole Blood Stability

The whole blood stability of the exemplified compounds is determined by LC-MS/MS. The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is pre-warmed in water bath to 37° C. for 30 min. 96-deep well analysis plate is prepared with the addition of 100 μL ultrapure water/well. 50 μL chilled ultrapure water/well is added to 96-deep well sample collection plate and covered with a sealing mat. 1 μL of 0.5 mM compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4-h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LC-MS/MS. The peak area ratio of the 1, 2, and 4 h samples relative to T=0 is used to determine the percent remaining. The natural log of the percent remaining versus time is used determine a slope to calculate the compounds half-life in blood (t_{1/2}=0.693/slope).

### Example F: In Vitro Intrinsic Clearance Protocol

For in vitro metabolic stability experiments, test compounds are incubated with human liver microsomes at 37° C. The incubation mixture contains test compounds (1 M), NADPH (2 mM), and human liver microsomes (0.5 mg protein/mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LC-MS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard is achieved using a Hypersil Gold C₁₈ column (50×2.1 mm, 5 μM, 175 A) from ThermoFisher Scientific (Waltham, MA). Mobile phase A consists of 0.1% formic acid in water, and mobile phase B consists of 0.1% formic acid in MeCN. The total LC-MS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calculated from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln2/k. The CL_{int, in vitro} values are scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)×(g liver weight/kg body weight) is used. The in vivo hepatic clearance (CLH) is then calculated by using CLᵢₙₜ and hepatic blood flow, Q (20 mL-min.⁻¹ kg⁻¹ in humans) in the well-stirred liver model disregarding all binding from CL_{H}=(Q×CLᵢₙₜ)/(Q+CLᵢₙₜ). The hepatic extraction ratio is calculated as CLH divided by Q.

### Example G: In Vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LC-MS methods. The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example H: Time Dependent Inhibition (TDI) of CYP Protocol

This assay is designed to characterize an increase in CYP inhibition as a test compounds is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8 min. in the presence of the substrate cocktail with the addition of a fresh aliquot of NADPH regenerating system. A calibration curve of metabolite standards can be used to quantitatively measure the enzyme activity using LC-MS/MS. In addition, incubations with known time dependent inhibitors, tienilic aicd (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard can be achieved using an ACQUITY UPLC BEH 130A, 2.1×50 mm, 1.7 m HPLC column (Waters Corp, Milford, MA). Mobile phase A consists of 0.1% formic acid in water, and mobile phase B consists of 0.1% formic acid in MeCN. The total LC-MS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calculated based on 0 min. as 100%. A linear regression plot of the natural log of % activity remaining versus time for each isozyme is used to calculate the slope. The −slope is equal to the rate of enzyme loss, or the K_{obs}.

### Example I: In Vitro Degranulation Assay—β-Hexosaminidase Release Assay

The assay is performed essentially as described by Hermans, et al., Front. Immunol., 2021, 12, 625284. Mature mast cells are cultured in StemPro-34 medium with StemPro-34 nutrient supplement (Gibco), 2 mM L-glutamine (Gibco), 100 U/mL penicillin/100 μg/mL streptomycin (Gibco), 100 ng/mL recombinant human stem cell factor (PeproTech) and 100 ng/mL recombinant human interleukin 6 (Peprotech). The cells are washed in HEPES-buffered Tyrode's solution (Thermo Fisher) containing 0.1% (w/v) bovine serum albumine (further called “releasing medium”) and seeded at 1×10⁵ cells per well in 96-well plate in the releasing medium. Selected wells are pretreated with predefined concentrations of MRGPRX2 antagonist for 1 h at 37° C. with 5% CO₂. Then, about 10 μg/mL compound 48/80 (poly-p-methoxyphenethylmethylamine) or about 30 μM of substance P is added to treated wells and equal amount of releasing medium is added into negative control wells. Following 1 h incubation, the plate is centrifuged for 5 min. and 50 μL of supernatant is collected into a new 96-well plate. In a positive control group, remaining supernatant is removed and 100 μL 0.1% Triton X-100 (v/v) is added to lyse the cell at 300 g for 5 min. After that, the plate is centrifuged and 50 μL of the lysate is also transferred into the new plate. Supernatant and lysate samples are then incubated with 50 μL of p-nitrophenyl N-acetyle-B-D-glucosaminide in 0.1 M citrate buffer pH 4-4.5 (Thermo Fisher) for 1 h at 37° C. The enzymatic reaction is stopped by the addition of 100 μL/well of 0.1 M Na₂CO₃ buffer pH 10 (Thermo Fisher). The absorbance is read at 405 nm. Net β-hexosaminidase release (%) is calculated as=[(stimulated release−spontaneous release)/total content in lysed cells]×100.

### Example J: Evaluation of the MRGPRX2 Antagonist Effects on In Vitro-Derived Human Mast Cells

This assay evaluates the effects of example compound on preventing mast cell degranulation via the MRGPRX2 receptor. The effects of example compounds on mast cell degranulation induced by Substance P (MRGPRX2 pathway), or IgE/anti-IgE is investigated by measuring 0-hexosaminidase and mast cell specific cytokine/chemokine release into the culture media. The addition of IgE/anti-IgE serves as a differentiation for the mechanism of action of the test compound.

Mature connective tissue-type mast cells (CTMCs) are plated at 50,000 cells by well in 50 μL and stimulated as follows.

### IgE Sensitization (Day 0)

All CTMCs are pre-treated with 100 μM IgE before treatment to coat the FcεR1 receptors with IgE to resemble in vitro mast cells. In this study, CTMCs are sensitized 48 h prior to pre-treatment with example compounds and degranulation (Day 2).

### Pre-Treatment (Day 0)

Omalizumab is added 4 h after IgE sensitization (IgE containing media is removed after 4 h and replaced with culture media containing omalizumab and kept for 48 h) and for the following 48 h before degranulation induction.

### Pre-Treatment (Day 2)

Test compounds and vehicles are added 48 h post IgE sensitization/omalizumab treatment for 30 min. at Day 2 of culture.

### Degranulation (Day 2+30 min.)

The CTMCs are stimulated with Substance P, Compound 48/80, anti-IgE, or Tyrode's buffer on Day 2 of culture.

### β-Hexosaminidase Assay to Assess Mast-Cell Degranulation

For the β-hexosaminidase assay, 50 μL of cell supernatant is collected in each well 45 min. after degranulation treatment. Supernatant is collected on n=3 wells per condition.

β-Hexosaminidase is a potent inflammatory mediator stored in mast cells and is released by activated mast cells. The determination of β-hexosaminidase is used to evaluate the level of mast cell degranulation. The assay is a colorimetric assay measuring the 4-nitrophenol production using a multimode plate reader. See J. Karhausen, et al., J. Clin. Invest., 2016, 126(10), 3981-98.

### Cytokine Release Assay

For the cytokine release assay, 60 μL of cell supernatant is collected in each well 8 h after degranulation treatment, an anti-protease cocktail added, and frozen at −80° C. until used. Supernatant is collected on n=3 wells per condition.

Cytokine release into the culture medium is measured using a custom V-PLEX Plus Human Cytokine Kit (MesoScale Discovery): TNF-α, IL-13, GM-CSF, VEGF-A, MCP-1, IL-6, IL-4, IL-5, IL-10, and IL-8. Culture media is diluted as follows:
Plate 1: Chemokine Panel 1 (human) kit—ref K15047 (panel MCP-1)—1:10;
Plate 2: Proinflammatory Panel 1 (human) kit—ref K15049 (panel IL-4, IL-6, IL-8, IL-10, IL-13, TNF-α)—1:5;
Plate 3: Cytokine Panel 1 (human) kit—ref K15050 (panel GM-CSF, IL-5, VEGF)—1:5.

### Example K: Determining the Effect of Example Compounds on Cytokine/Chemokine Release from HypoSkin Models after Subcutaneous Injection of Cetrorelix or Cortistatin-14

This assay evaluates the effect of the test compounds on cytokine/chemokine release (potentially from mast cells) upon systematic pre-treatment and treatment of HypoSkin models prior to subcutaneous injection of drugs known to degranulate mast cells and cause injection site reactions (namely, Cortistatin-14 and Cetrorelix).

74 HypoSkin models of 20 mm in diameter and 10 mm total thickness with 15/20 mm diameter silicon rings are produced from 2 donors (37 models per donor) according to standard procedures and cultured with 2 mL standard HypoSkin medium. Models are maintained in standard cell culture conditions for the whole culture duration at 37° C., 5% CO₂ and water saturation, with culture medium renewed every day except during weekends.

### Systematic Treatment

Vehicle control and test compounds are added to the culture media daily, from Day 0 to Day 2. The compounds remain in the culture media upon subcutaneous injections.

### Subcutaneous Injection

100 μL of Cetrorelix, Cortistatin-14, or PBS are subcutaneously injected in to the models on Day 2.

### End of Culture and Sampling

Culture media is collected on Days 2+8 h and Day 3 (24 h post-injection) and frozen at −80° C. Sampling of HypoSkin models is performed on Day 0 and Day 3 (24 h post-injection) and processed as follows:
½ are fixed in 10% buffered formalin and processed for paraffin wax embedding; and
½ are snap frozen and stored at −80° C.
   Hematoxylin and Eosin staining

Hematoxylin and Eosin staining is performed on one 5 μm thick skin cross section for each sample. Representative images of both the epidermis/dermis and hypodermis are taken at 40× magnification to analyze skin structure integrity and viability.

### MSD Immunoassay

Cytokine release in the culture medium is measuring using the V-PLEX Plus Human Cytokine 36-Plex Kit (K15089G, MesoScale Discovery): Eotaxin, Eotaxin-3, GM-CSF, IFN-γ, IL-1α, IL-1, 11-2, IL-4, IL-5, IL-6, IL-7, IL-8, IL-8 (HA), IL-10, IL12/IL-23p40, IL-23p70, IL-13, IL-15, IL-16, IL-17α, IL-21, IL-22, IL-23, IL-27, IL-31, IP-10, MCP-1, MCP-4, MDC, MIP-1α, MIP-1β, MIP-3α, TARC, TNF-α, TNF-β, and VEGF-A.

### Example L: Characterization of JAK Inhibitors MRGPRX2 Antagonists on Mast Cell Activation

The effect of the janus kinase inhibitors Povorcitinib, Ruxolitinib, and MRGPRX2 antagonists disclosed herein on the activation of mast cells in vitro is compared and characterized under different activating conditions, mimicking the in vivo disease situation. This assay determines the effect of JAK inhibition and MRGPRX2 antagonism on interference different mast cell activation pathways; the effect of JAK inhibition and MRGPRX2 antagonism in mast cells to prevent the release of mediators involved in the activation of primary human blood eosinophils and T cells.

Primary human skin mast cells (hsMC) are used to study how Povorcitinib, Ruxolitinib, and MRGPRX2 antagonists interfere with different prototype mast cell activators. The cells are obtained from individuals undergoing circumcision or breast reduction surgery by isolation from skin explants in a multi-step protocol. In vitro dose response studies using different concentration of the compounds to identify IC₅₀ values are conducted. For these studies, hsMCs are pre-incubated in the presence or absence of the compounds for 20 min., followed by stimulation with anti-IgE (FcεRI stimulation) or cortistatin-14 (MRGPRX2 agonist). Degranulation responses are measured by determination of β-hexosaminidase after 1 h at 37° C.

Next, three different concentrations of the compounds according to the determined IC₅₀ are employed to assess the spectrum of mast cell activating pathways inhibited by each compound. Therefore, hsMCs are pre-incubated in the presence or absence of the individual compounds for 20 min., followed by stimulation with five distinct activators: anti-IgE, corstatin-14 (CST), stem cell factor (SCF), complement peptides C3a, and C5a. In a parallel setup, hsMCs are pre-treated with various concentrations of cyclosporine A (CSA), to assess difference in efficacy to the test compounds. CSA, inhibiting the translocation of the transcription factor NFAT into the nucleus in MCs and other immune cells, is widely used to treat urticaria. As an alternative to CSA, another JAK or BTK inhibitor can be used. Degranulation responses are measured by determination of the β-hexosaminidase after 1 h at 37° C. In addition, cell viability and surface expression of FcεRI, MRGPRX2, cKitm C3aR/C5aR are measured by flow cytometry. Mast cells from three individual mast cell preparations are used.

To assess if treatment of mast cells with Povorcitinib, Ruxolitinib, or MRGPRX2 antagonists alters their mediator release profile and thereby their ability to activate eosinophils or T cells, hsMCs are pre-treated with the compounds for 20 min., followed by stimulation with anti-IgE, CST, SCF, C3a, or C5a for 1 h, 4 h, 8 h, and 24 h. Culture supernatants are collected and added to freshly isolated human peripheral blood eosinophils and total T cells. To achieve complete T cell activation, simultaneous activation of the TCR and costimulatory receptor with antibodies targeting CD3 and CD28 together with the mast cell supernatant is performed.

Cell activation is assessed using flow cytometry by measuring upregulation of CD69 and CD63 on eosinophils, as well as level of CD69, CD154, CD25, or CD62L on T cells. In addition, cytokines secreted by the mast cells are measured using bead-based cytokine multiplex assays (45 plex). Three individual experiments are performed using mast cells and eosinophils from three individual donors.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound having Formula (I): 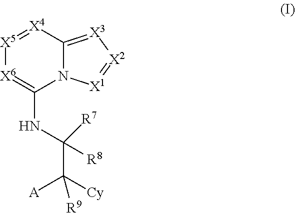
or a pharmaceutically acceptable salt thereof, wherein:
X¹ is N or CR¹;
X² is N or CR²;
X³ is N or CR³;
X⁴ is N or CR⁴;
X⁵ is N or CR⁵;
X⁶ is N or CR⁶;
R¹, R², R³, R⁴, R⁵, and R⁶ are each independently selected from H, D, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1} NR^{c1}R^{d1}, NR^{c1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1}, NRC(═NR^{c1})NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹, R², R³, R⁴, R⁵, and R⁶ are each optionally substituted with 1, 2, 3, or 4 substituents each independently selected from R^{10A}; and wherein the C₁₋₆ alkyl, C₂₋₆ alkenyl, and C₂₋₆ alkynyl forming R¹, R², R³, R⁴, R⁵, and R⁶ are each optionally substituted with 1, 2, 3, or 4 substituents each independently selected from R^{10B};
R⁷ and R⁸ are each independently selected from H, D, C₁₋₆ alkyl, C₂₋₆ alkenyl, C₂₋₆ alkynyl, C₁₋₆ haloalkyl, halo, C₃₋₁₀ cycloalkyl, and 4-10 membered heterocycloalkyl, wherein the C₃₋₁₀ cycloalkyl and 4-10 membered heterocycloalkyl forming R⁷ and R⁸ are each optionally substituted with 1, 2, 3, or 4 substituents independently selected from R^{20A}; and wherein the C₁₋₆ alkyl, C₂₋₆ alkenyl, and C₂₋₆ alkynyl forming R⁷ and R⁸ are each optionally substituted with 1, 2, 3, or 4 substituents independently selected from R^{20B};
R⁹ is selected from H, D, C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2} SR^{a2}, C(O)R^{b2}, C(O)NR^{c2}R^{d2}, C(O)OR^{a2}, OC(O)R^{b2}, OC(O)NR^{c2}R^{d2} NR^{c2}R^{d2} NR^{c2}C(O)R^{b2}, NR^{c2}C(O)OR^{a2} NR^{c2}C(O)NR^{c2}R^{d2}, C(═NR^{c2})R^{b2}, C(═NOR^{a2})R^{b2}, C(═NR^{c2})NR^{c2}R^{d2} NR^{c2}C(═NR^{c2})NR^{e2}R^{d2} NR^{c2}S(O)R^{b2} NR^{c2}S(O)₂R^{b2} NR^{c2}S(O)₂NR^{c2}R^{d2} S(O)R^{b2}, S(O)NR^{c2}R^{d2}, S(O)₂R^{b2}, and S(O)₂NR^{e2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁹ are each optionally substituted with 1, 2, 3, or 4 substituents independently selected from R^{30A}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{30B};
A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A};
Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3} NR^{c3}R^{d3} NR^{c3}C(O)R^{b3}, NR^{c3}C(O)OR^{a3} NR^{c3}C(O)NR^{c3}R^{d3}C(═NR^{c3})R^{b3}, C(═NOR^{a3})R^{b3}, C((═NR^{c3})NR^{c3}R^{d3} NR^{c3}C(═NR^{c3})NR³R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR³, SR^{a3}, C(O)R^{b3}, C(O)NR^{c3}R^{d3}, C(O)OR^{a}, NR^{c3}R^{d3} NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4} NR^{c4}R^{d4} NR^{c4}C(O)R^{b4}, NR^{c4}C(O)OR^{a4}, NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR^{c4})NR^{c4}R^{d4} NR^{c4}S(O)R^{b4} NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R^{g1}; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR^{e5}C(═NR⁵)NR^{c5}R^{d5} NR^{c5}S(O)R^{b5} NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{c6}C(O)R^{b6}NR^{c6}C(O)OR^{a6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6} S(O)₂R^{b6}, and S(O)₂NR^{c6}R^{d6}; wherein the C₃10 cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R⁹¹;
each R^{10A} is independently selected from C₁₋₆ alkyl, C₂₋₆ alkenyl, C₂₋₆ alkynyl, C₁₋₆ haloalkyl, and R^{10B}; wherein the C₁₋₆ alkyl, C₂₋₆ alkenyl, and C₂₋₆ alkynyl forming R^{10A} are each optionally substituted with 1, 2, 3, or 4 substituents independently selected from R^{10B};
each R^{10B} is independently selected from C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7} NR^{c7}R^{d7} NR^{c7}C(O)R^{b7} NR^{c7}C(O)OR^{a7} NR^{c7}C(O)NR^{c7}R^{d7}C(═NR^{c7})R^{b7}, C(═NOR^{a7})R^{b7}, C(═NR^{c7})NR^{c7}R^{d7} NR^{c7}C(═NR^{c7})NR^{c7}R^{d7} NR^{c7}S(O)R^{b7} NR^{c7}S(O)₂R^{b7} NR^{c7}S(O)₂NR^{a7}R^{d7}, S(O)R^{b7}, S(O)NR^{c7}R^{d7}, S(O)₂R^{b7}, and S(O)₂NR^{c7}R^{d7}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{10B} are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a8}, SR^{a8}, C(O)R^{b8}, C(O)NR^{c8}R^{d8}, C(O)OR^{a8}, NR^{c8}R^{d8} NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹¹ are each optionally substituted with 1, 2, 3, or 4 substituents independently selected from R^{g1}; and wherein the C₁₋₆ alkyl, C₂₋₆ alkenyl, and C₂₋₆ alkynyl forming R¹¹ are each optionally substituted with 1, 2, 3, or 4 substituents independently selected from R^{g2};
each R^{20A} is independently selected from C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{20A} are each optionally substituted with 1, 2, 3, or 4 substituents independently selected from R^{20B};
each R^{20B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a9}, SR^{a9}, C(O)R^{b9}, C(O)NR^{c9}R^{d9}, C(O)OR^{a9}, NR^{c9}R^{d9} NR^{c9}C(O)R^{d9} NR^{c9}C(O)OR^{a9} NR^{b9}S(O) NR^{c9}S(O)₂R^{d9}, NR^{c9}S(O)₂NR^{c9}R^{d9}, S(O)R^{d9}, S(O)NR^{c9}R^{d9} S(O)₂R^{b9}, and S(O)₂NR^{c9}R^{d9}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{20B} are each optionally substituted with 1, 2, 3, or 4 substituents independently selected from R^{g1};
each R^{30A} is independently selected from C₁₋₆ alkyl, C₂₋₆ alkenyl, C₂₋₆ alkynyl, C₁₋₆ haloalkyl, and R^{30B}; wherein the C₁₋₆ alkyl, C₂₋₆ alkenyl, and C₂₋₆ alkynyl forming R^{30A} are each optionally substituted with 1, 2, 3, or 4 substituents independently selected from R^{30B};
each R^{30B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a10}, SR^{a10}, C(O)R^{b10}, C(O)NR^{c10}R^{d10}, C(O)OR^{a10}, NR^{c10}R^{d10} NR^{c10}OC(O)R^{b10}, NR^{c10}C(O)OR^{a10}, NR^{c10}S(O)R^{b10}, NR^{c10}S(O)₂R^{b10}, NR^{c10}S(O)₂NR^{c10}R^{d10} S(O)R^{b10}, S(O)NR^{c10}R^{d10}, S(O)₂R^{b10}, and S(O)₂NR^{c10}R^{d10}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{30B} are each optionally substituted with 1, 2, 3, or 4 substituents independently selected from R^{g1};
each R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{b6}, R^{d6}, R^{a7}, R^{c7}, R^{d7}, R^{a8}, R^{c8}, R^{d8}, R^{a9}, R^{c9}, R^{d9}, R^{a10}, R^{c10} and R^{d10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a5}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} are each optionally substituted with 1, 2, 3, or 4 substituents independently selected from R^{g2};
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c8} and R^{d8} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c9} and R^{d9} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, R^{b3}, R^{b4}, R^{b5}, R^{b6}, R^{b7}, R^{b8}, R^{b9}, and R^{b10} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b1}, R^{b2}, R^{b3}, R^{b4}, R^{b5}, R^{b6}, R^{b7}, R^{b8}, R^{b9}, and R^{b10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b1}, R^{b2}, R^{b3}, R^{b4}, R^{b5}, R^{b6}, R^{b7}, R^{b8}, R^{b9} and R^{b10} are each optionally substituted with 1, 2, 3, or 4 substituents independently selected from R^{g2};
each R^{c1}, R^{c2}, R^{c3}, R^{c4}, R^{c5} and R^{c7} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2} wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}R^{d11} NR^{c11}C(O)R^{b11}, NR^{c11}C(O)OR^{a11}, NR^{c11}S(O)R^{b1}, NR^{c11}S(O)₂R^{b11}, NR^{c1}S(O)₂NR^{c11}R^{d11} S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c11}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a11}, R^{c11}, and R^{d11} are each optionally substituted with 1, 2, 3, or 4 substituents independently selected from R^{h2};
each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2};
each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
wherein at each occurrence, a heterocycloalkyl group has at least one ring-forming carbon atom and 1, 2, 3, or 4 ring-forming heteroatoms independently selected from N, O, and S; the N and S ring-forming heteroatoms of the heterocycloalkyl group are optionally oxidized; and a ring-forming carbon atom of the heterocycloalkyl group is optionally substituted by oxo to form a carbonyl group; and
at each occurrence, a heteroaryl group has at least one ring-forming carbon atom and 1, 2, 3, or 4 ring-forming heteroatoms independently selected from N, O, and S; the N and S ring-forming heteroatoms of the heteroaryl group are optionally oxidized; and a ring-forming carbon atom of the heteroaryl group is optionally substituted by oxo to form a carbonyl group;
with the proviso that the compound is not one of the following compounds:
N-[2-phenyl-2-(1-pyrrolidinyl)ethyl]tetrazolo[1,5-a]pyrazin-5-amine;
N-[2-(2-furanyl)-2-(1-pyrrolidinyl)ethyl]tetrazolo[1,5-a]pyrazin-5-amine;
N-[2-(5-methyl-2-furanyl)-2-(4-morpholinyl)ethyl]tetrazolo[1,5-a]pyrazin-5-amine;
N-[2-(4-methyl-1-piperidinyl)-2-(2-thienyl)ethyl][1,2,4]triazolo[1,5-a]pyrimidin-7-amine;
5-methyl-N-[2-phenyl-2-(4-piperidinyl)ethyl][1,2,4]triazolo[1,5-a]pyrimidin-7-amine;
5-methyl-N-[2-phenyl-2-(1-pyrrolidinyl)ethyl][1,2,4]triazolo[1,5-a]pyrimidin-7-amine;
N-[2-(2-furanyl)-2-(1-pyrrolidinyl)ethyl]-5-methyl[1,2,4]triazolo[1,5-a]pyrimidin-7-amine;
N-[2-(2-furanyl)-2-(1-piperidinyl)ethyl]-5-methyl[1,2,4]triazolo[1,5-a]pyrimidin-7-amine;
N-[2-(2-furanyl)-2-(4-morpholinyl)ethyl]-5-methyl[1,2,4]triazolo[1,5-a]pyrimidin-7-amine;
5-methyl-N-[2-(4-morpholinyl)-2-(2-thienyl)ethyl][1,2,4]triazolo[1,5-a]pyrimidin-7-amine;
5-methyl-N-[2-(4-morpholinyl)-2-(3-thienyl)ethyl][1,2,4]triazolo[1,5-a]pyrimidin-7-amine;
N-[2-(4-methoxyphenyl)-2-(1-pyrrolidinyl)ethyl]-5-methyl[1,2,4]triazolo[1,5-a]pyrimidin-7-amine;
5-methyl-N-[2-(5-methyl-2-furanyl)-2-(4-morpholinyl)ethyl][1,2,4]triazolo[1,5-a]pyrimidin-7-amine;
5-methyl-N-[2-(5-methyl-2-thienyl)-2-(4-morpholinyl)ethyl][1,2,4]triazolo[1,5-a]pyrimidin-7-amine;
5-methyl-N-[2-(2-methyl-4-morpholinyl)-2-(2-thienyl)ethyl][1,2,4]triazolo[1,5-a]pyrimidin-7-amine;
5-methyl-N-[2-(4-methylphenyl)-2-(4-morpholinyl)ethyl][1,2,4]triazolo[1,5-a]pyrimidin-7-amine;
5-methyl-N-[2-(3-methylphenyl)-2-(4-morpholinyl)ethyl][1,2,4]triazolo[1,5-a]pyrimidin-7-amine;
N-[2-(2-methoxyphenyl)-2-(1-pyrrolidinyl)ethyl]-5-methyl[1,2,4]triazolo[1,5-a]pyrimidin-7-amine;
N-[2-(4-methoxyphenyl)-2-(4-morpholinyl)ethyl]-5-methyl[1,2,4]triazolo[1,5-a]pyrimidin-7-amine;
N-[2-(4-fluorophenyl)-2-(4-morpholinyl)ethyl]-5-methyl[1,2,4]triazolo[1,5-a]pyrimidin-7-amine;
N-[2-(2-chlorophenyl)-2-(4-morpholinyl)ethyl]-5-methyl[1,2,4]triazolo[1,5-a]pyrimidin-7-amine;
N-[2-(4-methylphenyl)-2-(4-morpholinyl)ethyl]-5-phenyl[1,2,4]triazolo[1,5-a]pyrimidin-7-amine;
N-[2-(3,4-dimethoxyphenyl)-2-(4-morpholinyl)ethyl]-5-methyl[1,2,4]triazolo[1,5-a]pyrimidin-7-amine;
N-[2-(2-methoxyphenyl)-2-(1-piperidinyl)ethyl]imidazo[1,2-a]pyrimidin-5-amine;
5-ethyl-N-[2-(2-furanyl)-2-(1-pyrrolidinyl)ethyl]pyrazolo[1,5-a]pyrimidin-7-amine;
5-ethyl-N-[2-(5-methyl-2-furanyl)-2-(4-morpholinyl)ethyl]pyrazolo[1,5-a]pyrimidin-7-amine;
N-[2-(4-morpholinyl)-2-(3-pyridinyl)ethyl]-5-propylpyrazolo[1,5-a]pyrimidin-7-amine;
N-[2-(2-furanyl)-2-(1-pyrrolidinyl)ethyl]-2,3,5-trimethylpyrazolo[1,5-a]pyrimidin-7-amine;
5-(1-methylethyl)-N-[2-(5-methyl-2-furanyl)-2-(4-morpholinyl)ethyl]pyrazolo[1,5-a]pyrimidin-7-amine;
5-(1-methylethyl)-N-[2-(4-morpholinyl)-2-(4-pyridinyl)ethyl]pyrazolo[1,5-a]pyrimidin-7-amine;
5-(1-methylethyl)-N-[2-(4-morpholinyl)-2-(2-pyridinyl)ethyl]pyrazolo[1,5-a]pyrimidin-7-amine;
2,5-dimethyl-N-[2-(4-morpholinyl)-2-(4-pyridinyl)ethyl]pyrazolo[1,5-a]pyrimidin-7-amine;
2,5-dimethyl-N-[2-(4-morpholinyl)-2-(2-pyridinyl)ethyl]pyrazolo[1,5-a]pyrimidin-7-amine;
2,3,5-trimethyl-N-[2-(4-morpholinyl)-2-(3-pyridinyl)ethyl]pyrazolo[1,5-a]pyrimidin-7-amine;
N-[2-(4-morpholinyl)-2-(2-thienyl)ethyl]imidazo[1,2-c]pyrimidin-5-amine.

2. The compound or pharmaceutically acceptable salt thereof of claim 1, wherein the compound has Formula (I-A): 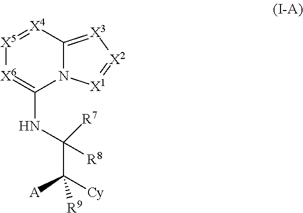
or a pharmaceutically acceptable salt thereof, wherein X¹, X², X³, X⁴, X⁵, X⁶, R⁷, R⁸, R⁹, A, and Cy are as defined in claim 1.

3. The compound or pharmaceutically acceptable salt thereof of claim 1, wherein the compound has Formula (I-B): 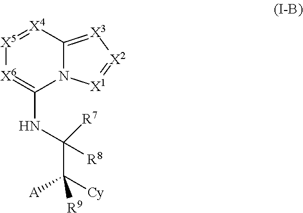
or a pharmaceutically acceptable salt thereof, wherein X¹, X², X³, X⁴, ×, X⁶, R⁷, R, R⁹, A, and Cy are as defined in claim 1.

4. The compound or pharmaceutically acceptable salt thereof of claim 1, wherein the compound has Formula (II-A), (II-B), (II-C), (II-D), (II-E), (II-F), (II-G), (II-H), (TI-I), (II-J), (TI-K), or (II-L): 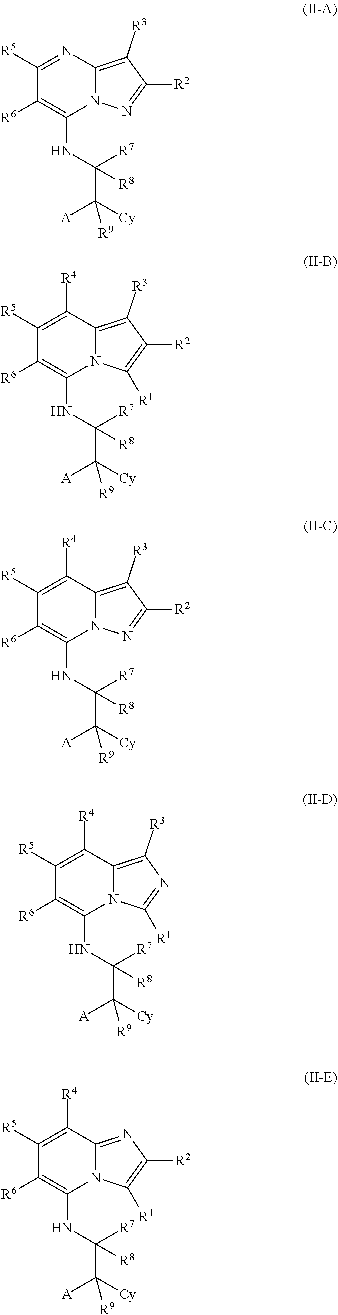 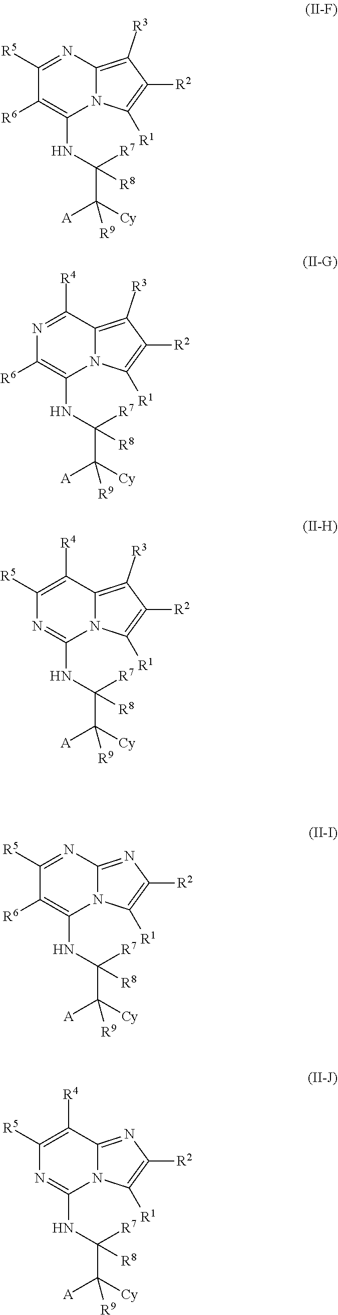 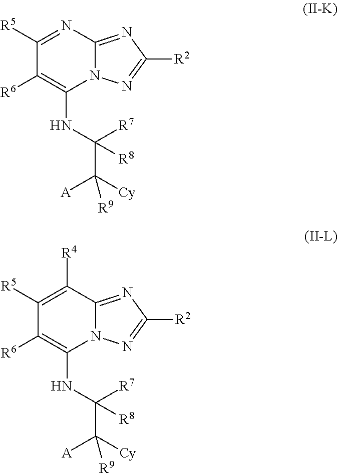
or a pharmaceutically acceptable salt thereof, wherein R¹, R², R³, R⁴, R⁵, R⁶, R⁷, R⁸, R⁹, Cy, and A are as defined in claim 1.

5. The compound or pharmaceutically acceptable salt thereof of claim 4, wherein the compound is other than a compound of Formula (II-A); the compound is other than a compound of Formula (II-B); the compound is other than a compound of Formula (II-C); the compound is other than a compound of Formula (II-D); the compound is other than a compound of Formula (II-E); the compound is other than a compound of Formula (II-F); the compound is other than a compound of Formula (II-G); the compound is other than a compound of Formula (II-H); the compound is other than a compound of Formula (II-I); the compound is other than a compound of Formula (II-J); the compound is other than a compound of Formula (II-K); and/or the compound is other than a compound of Formula (II-L); wherein Formulae (II-A), (II-B), (II-C), (II-D), (II-E), (II-F), (II-G), (II-H), (II-I), (II-J), (II-K), and (II-L) are as defined in claim 4.

6. The compound or pharmaceutically acceptable salt thereof of claim 1, wherein at least one of R¹, R², R³, R⁴, R⁵, and R⁶ is C₁₋₆ haloalkyl.

7. The compound or pharmaceutically acceptable salt thereof of claim 1, wherein X¹ is CR¹.

8. The compound or pharmaceutically acceptable salt thereof of claim 1, wherein X¹ is N.

9. The compound or pharmaceutically acceptable salt thereof of claim 1, wherein R¹ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}CR^{d1}, C(═NR^{c1})R^{b1}, C(═NOR^{a1})R^{b1}, C(═NR^{c1})NR^{c1}R^{d1} NR^{c1}C(═NR^{a1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, or P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

10. The compound or pharmaceutically acceptable salt thereof of claim 1, wherein R¹ is selected from H, D, C₁₋₆ alkyl, C₁₋₆ haloalkyl, halo, CN, and OR^{a1}; wherein the C₁₋₆ alkyl forming R¹ is optionally substituted with 1, 2, 3, or 4 substituents each independently selected from R^{10B}.

11. The compound or pharmaceutically acceptable salt thereof of claim 1, wherein R¹ is selected from H, D, C₁₋₆ alkyl (such as methyl or ethyl), C₁₋₆ haloalkyl (such as CF₃, CHF₂, CF₂CF₃), CN, and halo (such as F, Cl, or Br).

12. The compound or pharmaceutically acceptable salt thereof of claim 1, wherein R¹ is H.

13. The compound or pharmaceutically acceptable salt thereof of claim 1, wherein X² is CR².

14. The compound or pharmaceutically acceptable salt thereof of claim 1, wherein X² is N.

15. The compound or pharmaceutically acceptable salt thereof of claim 1, wherein R² is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}CR^{d1}, C(═NR^{c1})R^{b1}, C(═NOR^{a1})R^{b1}, C(═NR^{c1})NR^{c1}R^{d1} NR^{c1}C(═NR^{a1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R² are each optionally substituted with 1, 2, 3, or 4 substituents each independently selected from R^{10B}.

16. The compound or pharmaceutically acceptable salt thereof of claim 1, wherein R² is H, D, C₁₋₆ alkyl (such as methyl or ethyl), C₁₋₆ haloalkyl (such as CF₃, CHF₂, CF₂CF₃), CN, or halo (such as F, Cl, or Br).

17. The compound or pharmaceutically acceptable salt thereof of claim 1, wherein R² is C₁₋₆ alkyl, C₁₋₆ haloalkyl, or halo.

18. The compound or pharmaceutically acceptable salt thereof of claim 1, wherein R² is C₁₋₆ haloalkyl.

19. The compound or pharmaceutically acceptable salt thereof of claim 1, wherein R² is CF₃.

20. The compound or pharmaceutically acceptable salt thereof of claim 1, wherein X³ is CR³.

21. The compound or pharmaceutically acceptable salt thereof of claim 1, wherein X³ is N.

22. The compound or pharmaceutically acceptable salt thereof of claim 1, wherein R³ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{a1}C(O)R^{b1}, NR^{c1}C(O)OR^{a1}, NR^{c1}C(O)NR^{c1}R^{d1}, C(═NR^{c1})R^{b1}, C(═NOR^{a1})R^{b1}, C(═NR^{c1})NR^{c1}R^{d1}, NR^{c1}C(═NR^{c1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R³ are each optionally substituted with 1, 2, 3, or 4 substituents each independently selected from R^{10B}.

23. The compound or pharmaceutically acceptable salt thereof of claim 1, wherein R³ is selected from H, D, C₁₋₆ alkyl, C₁₋₆ haloalkyl, halo, CN, and OR^{a1}; wherein the C₁₋₆ alkyl forming R³ is optionally substituted with 1, 2, 3, or 4 substituents each independently selected from R^{10B}.

24. The compound or pharmaceutically acceptable salt thereof of claim 1, wherein R³ is H, D, C₁₋₆ alkyl (such as methyl or ethyl), C₁₋₆ haloalkyl (such as CF₃, CHF₂, CF₂CF₃), CN, or halo (such as F, Cl, or Br).

25. The compound or pharmaceutically acceptable salt thereof of claim 1, wherein R³ is H.

26. The compound or pharmaceutically acceptable salt thereof of claim 1, wherein X⁴ is CR⁴.

27. The compound or pharmaceutically acceptable salt thereof of claim 1, wherein X⁴ is N.

28. The compound or pharmaceutically acceptable salt thereof of claim 1, wherein R⁴ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1} NR^{a1}C(O)R^{b1}, NR^{c1}C(O)OR^{a1}, NR^{c1}C(O)NR^{c1}CR^{d1}, C(═NR^{c1})R^{b1}, C(═NOR^{a1})R^{b1}, C(═NR^{e1})NR^{c1}R^{d1} NR^{c1}C(═NR^{a1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R⁴ are each optionally substituted with 1, 2, 3, or 4 substituents each independently selected from R^{10B}.

29. The compound or pharmaceutically acceptable salt thereof of claim 1, wherein R⁴ is selected from H, D, C₁₋₆ alkyl, C₁₋₆ haloalkyl, halo, CN, and OR^{a1}; wherein the C₁₋₆ alkyl forming R⁴ is optionally substituted with 1, 2, 3, or 4 substituents each independently selected from R^{10B}.

30. The compound or pharmaceutically acceptable salt thereof of claim 1, wherein R⁴ is H, D, C₁₋₆ alkyl (such as methyl or ethyl), C₁₋₆ haloalkyl (such as CF₃, CHF₂, CF₂CF₃), CN, or halo (such as F, Cl, or Br).

31. The compound or pharmaceutically acceptable salt thereof of claim 1, wherein R⁴ is H.

32. The compound or pharmaceutically acceptable salt thereof of claim 1, wherein X⁵ is CR⁵.

33. The compound or pharmaceutically acceptable salt thereof of claim 1, wherein X⁵ is N.

34. The compound or pharmaceutically acceptable salt thereof of claim 1, wherein R⁵ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{a1}C(O)R^{b1}, NR^{c1}C(O)OR^{a1}, NR^{c1}C(O)NR^{c1}CR^{d1}, C(═NR^{c1})R^{b1}, C(═NOR^{a1})R^{b1}, C(═NR^{c1})NR^{c1}R^{d1} NR^{c1}C(═NR^{a1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R⁵ are each optionally substituted with 1, 2, 3, or 4 substituents each independently selected from R^{10B}.

35. The compound or pharmaceutically acceptable salt thereof of claim 1, wherein R⁵ is H, D, C₁₋₆ alkyl (such as methyl or ethyl), C₁₋₆ haloalkyl (such as CF₃, CHF₂, CF₂CF₃), CN, or halo (such as F, Cl, or Br).

36. The compound or pharmaceutically acceptable salt thereof of claim 1, wherein R⁵ is C₁₋₆ haloalkyl.

37. The compound or pharmaceutically acceptable salt thereof of claim 1, wherein R⁵ is CF₃ or CHF₂.

38. The compound or pharmaceutically acceptable salt thereof of claim 1, wherein X⁶ is CR⁶.

39. The compound or pharmaceutically acceptable salt thereof of claim 1, wherein X⁶ is N.

40. The compound or pharmaceutically acceptable salt thereof of claim 1, wherein R⁶ is selected from H, D, C₁₋₆ alkyl, C₂₋₆ alkenyl, C₂₋₆ alkynyl, C₁₋₆ haloalkyl, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}CR^{d1}, C(═NR^{c1})R^{b1}, C(═NOR^{a1})R^{b1}, C(═NR^{c1})NR^{c1}R^{d1} NR^{c1}C(═NR^{a1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{b1}, and P(O)(NH₂)OR^{a1}; wherein the C₁₋₆ alkyl, C₂₋₆ alkenyl, and C₂₋₆ alkynyl forming R⁶ are each optionally substituted with 1, 2, 3, or 4 substituents each independently selected from R^{10B}.

41. The compound or pharmaceutically acceptable salt thereof of claim 1, wherein R⁶ is selected from H, D, C₁₋₆ alkyl, C₁₋₆ haloalkyl, halo, CN, and OR^{a1}; wherein the C₁₋₆ alkyl forming R⁶ is optionally substituted with 1, 2, 3, or 4 substituents each independently selected from R^{10B}.

42. The compound or pharmaceutically acceptable salt thereof of claim 1, wherein R⁶ is H, D, C₁₋₆ alkyl (such as methyl or ethyl), C₁₋₆ haloalkyl (such as CF₃, CHF₂, CF₂CF₃), CN, or halo (such as F, Cl, or Br).

43. The compound or pharmaceutically acceptable salt thereof of claim 1, wherein R⁶ is H.

44. The compound or pharmaceutically acceptable salt thereof of claim 1, wherein R⁷ and R⁸ are each independently selected from H, D, C₁₋₆ alkyl, C₁₋₆ haloalkyl, and halo; wherein the C₁₋₆ alkyl forming R⁷ and R⁸ are each optionally substituted with 1, 2, 3, or 4 substituents each independently selected from R^{20B}.

45. The compound or pharmaceutically acceptable salt thereof of claim 1, wherein R⁷ and R⁸ are each independently selected from H, D, C₁₋₆ alkyl (such as methyl or ethyl), C₁₋₆ haloalkyl (such as CF₃, CHF₂, CF₂CF₃), and halo (such as F, Cl, or Br).

46. The compound or pharmaceutically acceptable salt thereof of claim 1, wherein R⁷ and R⁸ are each H.

47. The compound or pharmaceutically acceptable salt thereof of claim 1, wherein R⁹ is selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2} SR^{a2}, C(O)R^{b2}, C(O)NR^{c2}R^{d2}, C(O)OR^{a2}, OC(O)R^{b2}, OC(O)NR^{c2}R^{d2} NR^{c2}R^{d2} NR^{c2}C(O)R^{b2} NR^{c2}C(O)OR^{a2} NR^{c2}C(O)NR^{c2}R^{d2}, C(═NR^{c2})R^{b2}, C(═NOR^{a2})R^{b2}, C(═NR^{c2})NR^{c2}R^{d2} NR^{c2}C(═NR^{c2})R^{c2}R^{d2} NR^{c2}S(O)R^{b2}, NR^{c2}S(O)₂R^{b2} NR^{e2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁹ are each optionally substituted with 1, 2, 3, or 4 substituents independently selected from R^{30A}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{30B}.

48. The compound or pharmaceutically acceptable salt thereof of claim 1, wherein R⁹ is selected from H, D, C₁₋₆ alkyl, C₁₋₆ haloalkyl, halo, CN, and OR^{a2}; wherein the C₁₋₆ alkyl forming R⁹ is optionally substituted with 1, 2, 3, or 4 substituents each independently selected from R^{30B}.

49. The compound or pharmaceutically acceptable salt thereof of claim 1, wherein R⁹ is H, D, C₁₋₆ alkyl (such as methyl or ethyl), C₁₋₆ haloalkyl (such as CF₃, CHF₂, CF₂CF₃), CN, or halo (such as F, Cl, or Br).

50. The compound or pharmaceutically acceptable salt thereof of claim 1, wherein R⁹ is selected from H and C₁₋₆ alkyl, wherein the C₁₋₆ alkyl forming R⁹ is optionally substituted with OR^{a10}.

51. The compound or pharmaceutically acceptable salt thereof of claim 1, wherein R⁹ is C₁₋₆ alkyl.

52. The compound or pharmaceutically acceptable salt thereof of claim 1, wherein R⁹ is methyl or ethyl.

53. The compound or pharmaceutically acceptable salt thereof of claim 1, wherein R⁹ is selected from H, methyl, ethyl, and CH₂OH.

54. The compound or pharmaceutically acceptable salt thereof of claim 1, wherein A is optionally substituted with 1, 2, 3, 4, 5 or 6 substituents independently selected from R^{A}.

55. The compound or pharmaceutically acceptable salt thereof of claim 1, wherein A is optionally substituted with 1, 2, 3, 4, or 5 substituents independently selected from R^{A}.

56. The compound or pharmaceutically acceptable salt thereof of claim 1, wherein A is optionally substituted with 1, 2, 3, or 4 substituents independently selected from R^{A}.

57. The compound or pharmaceutically acceptable salt thereof of claim 1, wherein A is optionally substituted with 1, 2, or 3 substituents independently selected from R^{A}.

58. The compound or pharmaceutically acceptable salt thereof of claim 1, wherein A is optionally substituted with 1 or 2 substituents independently selected from R^{A}.

59. The compound or pharmaceutically acceptable salt thereof of claim 1, wherein A is optionally substituted with 1 substituent selected from R^{A}.

60. The compound or pharmaceutically acceptable salt thereof of claim 1, wherein A is unsubstituted.

61. The compound or pharmaceutically acceptable salt thereof of claim 1, wherein A is 5-10 membered heterocycloalkyl optionally substituted with 1, 2, 3, 4, 5 or 6 substituents independently selected from R^{A}.

62. The compound or pharmaceutically acceptable salt thereof of claim 1, wherein A is 5-10 membered heterocycloalkyl optionally substituted with 1, 2, 3, 4, or 5 substituents independently selected from R^{A}.

63. The compound or pharmaceutically acceptable salt thereof of claim 1, wherein A is 5-10 membered heterocycloalkyl optionally substituted with 1, 2, 3, or 4 substituents independently selected from R^{A}.

64. The compound or pharmaceutically acceptable salt thereof of claim 1, wherein A is 5-10 membered heterocycloalkyl optionally substituted with 1, 2, or 3 substituents independently selected from R^{A}.

65. The compound or pharmaceutically acceptable salt thereof of claim 1, wherein A is 5-10 membered heterocycloalkyl optionally substituted with 1 or 2 substituents independently selected from R^{A}.

66. The compound or pharmaceutically acceptable salt thereof of claim 1, wherein A is 5-10 membered heterocycloalkyl optionally substituted with 1 substituent selected from R^{A}.

67. The compound or pharmaceutically acceptable salt thereof of claim 1, wherein A is unsubstituted 5-10 membered heterocycloalkyl.

68. The compound or pharmaceutically acceptable salt thereof of claim 1, wherein A is selected from azetidinyl, piperidinyl, and pyrrolidinyl, each of which is unsubstituted or substituted with 1, 2, or 3 substituents independently selected from R^{A}.

69. The compound or pharmaceutically acceptable salt thereof of claim 1, wherein A is selected from azetidinyl, piperidinyl, pyrrolidinyl, 2-azabicyclo[2.2.1]heptanyl, 2-azaspiro[3.5]nonan-2-yl, 6-oxo-7-oxa-2,5-diazaspiro[3.5]nonanyl, 2,6-diazaspiro[3.3]heptanyl, 6-oxo-2,5-diazaspiro[3.4]octanyl, 7-oxo-2,6-diazaspiro[3.4]octanyl, 2-azaspiro[3.3]heptanyl, 6-oxo-2,5,7-triazaspiro[3.4]octanyl, and 6-oxo-7-oxa-2,5-diazaspiro[3.4]octan-2-yl, each of which is unsubstituted or substituted with 1, 2, or 3 substituents independently selected from R^{A}.

70. The compound or pharmaceutically acceptable salt thereof of claim 1, wherein each R^{A} is independently selected from 5-10 membered heteroaryl, C₁₋₆ alkyl, C₂₋₆ alkenyl, C₂₋₆ alkynyl, C₁₋₆ haloalkyl, halo, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3} NR^{c3}R^{d3} NR^{c3}C(O)R^{b3}, NR^{c3}C(O)OR^{a3}, NR^{c3}C(O)NR^{c3}R^{d3}, C(═NR^{c3})R^{b3}, C(═NOR^{a3})R^{b3}, C(═NR^{c3})NR^{c3}R^{d3} NR^{c3}C(═NR^{c3})NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3}; wherein the 5-10 membered heteroaryl is optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2}.

71. The compound or pharmaceutically acceptable salt thereof of claim 1, wherein each R^{A} is selected from C₁₋₆ alkyl (such as methyl or ethyl), C₁₋₆ haloalkyl (such as CF₃, CHF₂, CF₂CF₃), halo (such as F, Cl, or Br), and OR^{a3} (such as methoxy or ethoxy).

72. The compound or pharmaceutically acceptable salt thereof of claim 1, wherein each R^{A} is selected from 5-10 membered heteroaryl, C(O)NR^{c3}R^{d3}, S(O)₂NR^{c3}R^{d3}, and C(═NR^{e3})NR^{c3}R^{d3} wherein 5-10 membered heteroaryl is optionally substituted with R^{A1}.

73. The compound or pharmaceutically acceptable salt thereof of claim 1, wherein each R^{A} is selected from 5-10 membered heteroaryl, C₁₋₆ alkyl, OR^{a3}, NR^{c3}R^{d3}, C(O)R^{b3}, C(O)NR^{c3}R^{d3} S(O)₂NR³R^{d3}, and C(═NR^{e3})NR^{c3}R^{d3}, wherein the 5-10 membered heteroaryl forming R^{A} is optionally substituted with R^{A1} and the C₁₋₆ alkyl forming R^{A} is optionally substituted with R^{A}.

74. The compound or pharmaceutically acceptable salt thereof of claim 1, wherein each R^{A} is selected from C(O)NH₂, S(O)₂NH₂, C(═NCN)NH₂, 5-amino-1H-1,2,4-triazol-3-yl, and 1-methyl-6-oxo-1,6-dihydropyridazin-3-yl.

75. The compound or pharmaceutically acceptable salt thereof of claim 1, wherein each R^{A} is selected from methyl, OH, C(O)NH₂, S(O)₂NH₂, C(═NCN)NH₂, C(O)N(H)CD₃, CH₂OH, pyridinyl, 5-amino-1H-1,2,4-triazol-3-yl, and 1-methyl-6-oxo-1,6-dihydropyridazin-3-yl.

76. The compound or pharmaceutically acceptable salt thereof of claim 1, wherein each R^{A} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2}.

77. The compound or pharmaceutically acceptable salt thereof of claim 1, wherein each R^{A} is independently selected from C₁₋₆ alkyl and R^{A2}.

78. The compound or pharmaceutically acceptable salt thereof of claim 1, wherein each R^{A2} is independently selected from halo, CN, OR^{a3}, SR^{a3}, C(O)R^{b3}, C(O)NR^{c3}R^{d3}, C(O)OR^{a3}, NR^{c3}R^{d3} NR^{c3}C(O)R^{b3}, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

79. The compound or pharmaceutically acceptable salt thereof of claim 1, wherein R^{A2} is NR^{c3}R^{d3}.

80. The compound or pharmaceutically acceptable salt thereof of claim 1, wherein each R^{A2} is independently selected from NR^{c3}R^{d3}, OR^{a3}, and C(O)NR^{c3}R^{d3}.

81. The compound or pharmaceutically acceptable salt thereof of claim 1, wherein Cy is substituted with 1, 2, 3, 4, 5 or 6 substituents independently selected from R^{Cy}.

82. The compound or pharmaceutically acceptable salt thereof of claim 1, wherein Cy is substituted with 1, 2, 3, 4, or 5 substituents independently selected from R^{Cy}.

83. The compound or pharmaceutically acceptable salt thereof of claim 1, wherein Cy is substituted with 1, 2, 3, or 4 substituents independently selected from R^{Cy}.

84. The compound or pharmaceutically acceptable salt thereof of claim 1, wherein Cy is substituted with 1, 2, or 3 substituents independently selected from R^{Cy}.

85. The compound or pharmaceutically acceptable salt thereof of claim 1, wherein Cy is substituted with 1 or 2 substituents independently selected from R^{Cy}.

86. The compound or pharmaceutically acceptable salt thereof of claim 1, wherein Cy is substituted with 1 substituent selected from R^{Cy}.

87. The compound or pharmaceutically acceptable salt thereof of claim 1, wherein Cy is unsubstituted.

88. The compound or pharmaceutically acceptable salt thereof of claim 1, wherein Cy is phenyl substituted with 1, 2, 3, 4, or 5 substituents independently selected from R^{Cy}.

89. The compound or pharmaceutically acceptable salt thereof of claim 1, wherein Cy is phenyl substituted with 1, 2, 3, or 4 substituents independently selected from R^{Cy}.

90. The compound or pharmaceutically acceptable salt thereof of claim 1, wherein Cy is phenyl substituted with 1, 2, or 3 substituents independently selected from R^{Cy}.

91. The compound or pharmaceutically acceptable salt thereof of claim 1, wherein Cy is phenyl substituted with 1 or 2 substituents independently selected from R^{Cy}.

92. The compound or pharmaceutically acceptable salt thereof of claim 1, wherein Cy is phenyl substituted with 1 substituent selected from R^{Cy}.

93. The compound or pharmaceutically acceptable salt thereof of claim 1, wherein Cy is unsubstituted phenyl.

94. The compound or pharmaceutically acceptable salt thereof of claim 1, wherein Cy is selected from 4-halophenyl and phenyl.

95. The compound or pharmaceutically acceptable salt thereof of claim 1, wherein Cy is selected from 4-fluorophenyl and phenyl.

96. The compound or pharmaceutically acceptable salt thereof of claim 1, wherein each R^{Cy} is independently selected from C₁₋₆ alkyl, C₂₋₆ alkenyl, C₂₋₆ alkynyl, C₁₋₆ haloalkyl, halo, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5}, NR^{c5}C(O)NR^{c5}R^{d5}, C(═NR^{c5})R^{d5}, C(═NOR^{a5})R^{b5}, C(═NR^{e5})NR^{c5}R^{d5} NR^{b5}C(═NR^{c5})NR^{c5}R^{d5} NR^{c5}S(O)R^{b5}, NR^{c5}S(O)₂R¹, NR^{c5}S(O)₂NR^{c5}R^{d5} S(O)R^{b5}, S(O)NR^{c5}R^{d5}, S(O)₂R^{b5}, and S(O)₂NR^{c5}R^{d5}; wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2}.

97. The compound or pharmaceutically acceptable salt thereof of claim 1, wherein each R^{Cy} is selected from C₁₋₆ alkyl (such as methyl or ethyl), C₁₋₆ haloalkyl (such as CF₃, CHF₂, CF₂CF₃), halo (such as F, Cl, or Br), and OR^{a2} (such as methoxy or ethoxy).

98. The compound or pharmaceutically acceptable salt thereof of claim 1, wherein each R^{Cy} is selected from halo.

99. The compound or pharmaceutically acceptable salt thereof of claim 1, wherein each R^{Cy} is F.

100. The compound or pharmaceutically acceptable salt thereof of claim 1, wherein the compound has Formula (II-A): 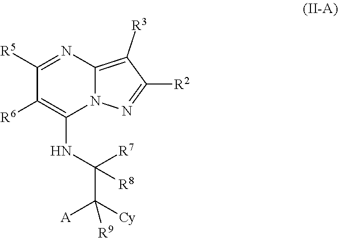
or a pharmaceutically acceptable salt thereof, wherein:
R², R³, R⁵, and R⁶ are each independently selected from H, D, C₁₋₆ alkyl, C₂₋₆ alkenyl, C₂-6 alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a1} SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}CR^{d1}, C(═NR^{c1})R^{b1}, C(═NOR^{a1})R^{b1}, C(═NR^{c1})NR^{c1}R^{d1} NR^{c1}C(═NR^{c1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, S(O)(═NH)R^{b1}, S(O)₂NR^{c1}R^{d1}, P(O)(NH₂)R^{a1}, or P(O)(NH₂)OR^{a1}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R², R³, R⁵, and R⁶ are each optionally substituted with 1, 2, 3, or 4 substituents each independently selected from R^{10A}; and wherein the C₁₋₆ alkyl, C₂₋₆ alkenyl, and C₂₋₆ alkynyl forming R², R³, R⁵, and R⁶ are each optionally substituted with 1, 2, 3, or 4 substituents each independently selected from R^{10B};
R⁷ and R⁸ are each independently selected from H, D, C₁₋₆ alkyl, C₂₋₆ alkenyl, C₂₋₆ alkynyl, C₁₋₆ haloalkyl, halo, C₃₋₁₀ cycloalkyl, and 4-10 membered heterocycloalkyl, wherein the C₃₋₁₀ cycloalkyl and 4-10 membered heterocycloalkyl forming R⁷ and R⁸ are each optionally substituted with 1, 2, 3, or 4 substituents independently selected from R^{20A}; and wherein the C₁₋₆ alkyl, C₂₋₆ alkenyl, and C₂₋₆ alkynyl forming R⁷ and R⁸ are each optionally substituted with 1, 2, 3, or 4 substituents independently selected from R^{20B};
R⁹ is selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a2} SR^{a2}, C(O)R^{b2}, C(O)NR^{c2}R^{d2}, C(O)OR^{a2}, OC(O)R^{b2}, OC(O)NR^{c2}R^{d2} NR^{c2}R^{d2} NR^{c2}C(O)R^{b2}, NR^{c2}C(O)OR^{a2}, NR^{c2}C(O)NR^{c2}R^{d2}, C(═NR^{c2})R^{b2}, C(═NOR^{a2})R^{b2}, C(═NR^{c2})NR^{c2}R^{d2} NR^{c2}C(═NR^{c2})R^{c2}R^{d2} NR^{c2}S(O)R^{b2} NR^{c2}S(O)₂R^{b2} NR^{c2}S(O)₂NR^{c2}R^{d2} S(O)R^{b2}, S(O)NR^{c2}R^{d2}, S(O)₂R^{b2}, and S(O)₂NR^{c2}R^{d2}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R⁹ are each optionally substituted with 1, 2, 3, or 4 substituents independently selected from R^{30A}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{30B};
A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A};
Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR³, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{c3})R^{b3}, C(═NOR^{a3})R^{b3}, C(═NR^{c3})NR^{c3}R^{d3}, NR^{c3}C(═NR^{c3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR³, SR^{a3}, C(O)R^{b3}, C(O)NR^{c3}R^{d3}, C(O)OR^{a}, NR^{c3}R^{d3} NR^{c3}C(O)R^{b3}NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4} NR^{c4}R^{d4} NR^{c4}C(O)R^{b4}, NR^{c4}C(O)OR^{a4} NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR⁴)NR^{c4}R^{d4} NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R^{g1}; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR⁵C(═NR⁵)NR^{c5}R^{d5} NR^{c5}S(O)R^{b5} NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{c6}C(O)R^{b6}NR^{c6}C(O)OR^{a6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6} S(O)₂R^{b6}, and S(O)₂NR^{c6}R^{d6}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R⁹¹;
each R^{10A} is independently selected from C₁₋₆ alkyl, C₂₋₆ alkenyl, C₂₋₆ alkynyl, C₁₋₆ haloalkyl, and R^{10B}; wherein the C₁₋₆ alkyl, C₂₋₆ alkenyl, and C₂₋₆ alkynyl forming R^{10A} are each optionally substituted with 1, 2, 3, or 4 substituents independently selected from R^{10B};
each R^{10B} is independently selected from C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7} NR^{c7}R^{d7} NR^{c7}C(O)R^{b7} NR^{c7}C(O)OR^{a7} NR^{c7}C(O)NR^{c7}R^{d7}C(═NR^{c7})R^{b7}, C(═NOR^{a7})R^{b7}, C(═NR^{c7})NR^{c7}R^{d7} NR^{c7}C(═NR^{c7})NR^{c7}R^{d7} NR^{c7}S(O)R^{b7} NR^{c7}S(O)₂R^{b7} NR^{c7}S(O)₂NR^{c7}R^{d7}, S(O)R^{b7}, S(O)NR^{c7}R^{d7}, S(O)₂R^{b7}, and S(O)₂NR^{c7}R^{d7}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{10B} are each optionally substituted with 1, 2, 3, or 4 substituents independently selected from R¹¹;
each R¹¹ is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a8}, SR^{a8}, C(O)R^{b8}, C(O)NR^{c8}R^{d8}, C(O)OR^{a8}, NR^{c8}R^{d8} NR^{c8}C(O)R^{b8}, NR^{c8}C(O)OR^{a8}, NR^{c8}S(O)R^{b8}, NR^{c8}S(O)₂R^{b8}, NR^{c8}S(O)₂NR^{c8}R^{d8}, S(O)R^{b8}, S(O)NR^{c8}R^{d8}, S(O)₂R^{b8}, and S(O)₂NR^{c8}R^{d8}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R¹¹ are each optionally substituted with 1, 2, 3, or 4 substituents independently selected from R^{g1}; and wherein the C₁₋₆ alkyl, C₂₋₆ alkenyl, and C₂₋₆ alkynyl forming R¹¹ are each optionally substituted with 1, 2, 3, or 4 substituents independently selected from R^{g2};
each R^{20A} is independently selected from C₁₋₆ alkyl, C₂₋₆ alkenyl, C₂₋₆ alkynyl, and C₁₋₆ haloalkyl; wherein the C₁₋₆ alkyl, C₂₋₆ alkenyl, and C₂₋₆ alkynyl forming R^{20A} are each optionally substituted with 1, 2, 3, or 4 substituents independently selected from R^{20B};
each R^{20B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a9}, SR^{a9}, C(O)R^{b9}, C(O)NR^{c9}R^{d9}, C(O)OR^{a9}, NR^{c9}R^{d9} NR^{c9}C(O)R^{b9}, NR^{c9}C(O)OR^{a9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9} S(O)₂R^{b9}, and S(O)₂NR^{c9}R^{d9}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{20B} are each optionally substituted with 1, 2, 3, or 4 substituents independently selected from R^{g1};
each R^{30A} is independently selected from C₁₋₆ alkyl, C₂₋₆ alkenyl, C₂₋₆ alkynyl, C₁₋₆ haloalkyl, and R^{30B}; wherein the C₁₋₆ alkyl, C₂₋₆ alkenyl, and C₂₋₆ alkynyl forming R^{30A} are each optionally substituted with 1, 2, 3, or 4 substituents independently selected from R^{30B};
each R^{30B} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a10}, SR^{a10}, C(O)R^{b10}, C(O)NR^{c10}R^{d10}, C(O)OR^{a10}, NR^{c10}R^{d10} NR^{c10}C(O)R^{b10}, NR^{c10}C(O)OR^{a10}, NR^{c10}S(O)R^{b10}, NR^{c10}S(O)₂R^{b10}NR^{c10}S(O)₂NR^{c10}R^{d10} S(O)R^{b10}, S(O)NR^{c10}R^{d10}, S(O)₂R^{b10}, and S(O)₂NR^{c10}R^{d10}; wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{30B} are each optionally substituted with 1, 2, 3, or 4 substituents independently selected from R^{g1};
each R^{a1}, R^{c1}, R^{d1}, R^{a2}, R^{c2}, R^{d2}, R^{a3}, R^{c3}, R^{d3}, R^{a4}, R^{c4}, R^{d4}, R^{a8}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a8}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10} and R^{d10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a1}, R^{c1}, R^{d1}, R^{a2}, R^{c2}, R^{d2}, R^{a3}, R^{c3}, R^{d3}, R^{a4}, R^{c4}, R^{d4}, R^{a8}, R^{c5}, R^{d5}, R^{a6}, R^{c6}, R^{d6}, R^{a7}, R^{c7}, R^{d7}, R^{a8}, R^{c8}, R^{d8}, R^{a9}, R^{c9}, R^{d9}, R^{a10}, R^{c10}, and R^{d10} are each optionally substituted with 1, 2, 3, or 4 substituents independently selected from R^{g2};
or any R^{c1} and R^{d1} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c2} and R^{d2} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c7} and R^{d7} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c8} and R^{d8} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c9} and R^{d9} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c10} and R^{d10} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b1}, R^{b2}, R^{b3}, R^{b4}, R^{b5}, R^{b6}, R^{b7}, R^{b8}, R^{b9}, and R^{b10} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b1}, R^{b2}, R^{b3}, R^{b4}, R^{b5}, R^{b6}, R^{b7}, R^{b8}, R^{b9}, and R^{c10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b1}, R^{b2}, R^{b3}, R^{b4}, R^{b5}, R^{b6}, R^{b7}, R^{b8}, R^{b9}, and R^{b10} are each optionally substituted with 1, 2, 3, or 4 substituents independently selected from R^{g2};
each R^{c1}, R^{c2}, R^{c3}, R^{c4}, R^{c5} and R^{c7} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2} wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}R^{d11}NR^{c11}C(O)R^{b11}, NR^{c11}C(O)OR^{a11}, NR^{c11}S(O)R^{b11}, NR^{c11}S(O)₂R^{b11} NR^{c11}S(O)₂NR^{c11}R^{d11} S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a11}, R^{c11}, and R^{d11} are each optionally substituted with 1, 2, 3, or 4 substituents independently selected from R^{h2};
each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2};
each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

101. The compound or pharmaceutically acceptable salt thereof of claim 1, wherein the compound has Formula (II-A): 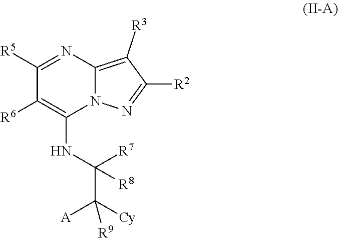
or a pharmaceutically acceptable salt thereof, wherein:
R², R³, R⁵, and R⁶ are each independently selected from H, D, C₁₋₆ alkyl, C₁₋₆ haloalkyl, CN, or halo;
R⁷ and R⁸ are each independently selected from H, D, C₁₋₆ alkyl, C₁₋₆ haloalkyl, CN, or halo;
R⁹ is selected from H, D, C₁₋₆ alkyl, and C₁₋₆ haloalkyl, wherein the C₁₋₆ alkyl forming R⁹ is optionally substituted with OR^{a10};
A is a ring selected from C₃₋₁₂ cycloalkyl and 4-12 membered heterocycloalkyl, wherein the C₃₋₁₂ cycloalkyl and 4-12 membered heterocycloalkyl forming A are each optionally substituted with 1, 2, 3, 4, 5, or 6 substituents independently selected from R^{A};
Cy is a ring selected from C₆₋₁₀ aryl and 5-10 membered heteroaryl; wherein the C₆₋₁₀ aryl and 5-10 membered heteroaryl forming Cy are each optionally substituted with 1, 2, 3, 4, or 5 substituents independently selected from R^{Cy};
each R^{A} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a}, SR^{a3}, C(O)R^{b3}, C(O)NR^{c3}R^{d3}, C(O)OR^{a3}, OC(O)R^{b3}, OC(O)NR^{c3}R^{d3} NR^{c3}R^{d3} NR^{c3}C(O)R^{b3}, NR^{c3}C(O)OR^{a3} NR^{c3}C(O)NR^{c3}R^{d3}C(═NR^{c3})R^{b3}, C(═NOR^{a3})R^{b3}, C(═NR^{c3})NR^{c3}R^{d3} NR^{c3}C(═NR^{c3})NR^{b3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A} are each optionally substituted with 1, 2, 3, or 4 substituents independently selected from R^{A1}; and wherein the C₁₋₆ alkyl, C₂₋₆ alkenyl, and C₂₋₆ alkynyl forming R^{A} are each optionally substituted with 1, 2, 3, or 4 substituents independently selected from R^{A2};
each R^{A1} is independently selected from C₁₋₆ alkyl, C₂₋₆ alkenyl, C₂₋₆ alkynyl, C₁₋₆ haloalkyl, and R^{A2}; wherein the C₁₋₆ alkyl, C₂₋₆ alkenyl, and C₂₋₆ alkynyl forming R^{A1} are each optionally substituted with 1, 2, 3, or 4 substituents independently selected from R^{A2};
each R^{A2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a3}, SR^{a3}, C(O)R^{b3}, C(O)NR^{c3}R^{d3}, C(O)OR^{a3}, NR^{c3}R^{d33} NR^{c3}C(O)R^{b}3, NR^{c3}C(O)OR^{a3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, S(O)(═NR^{c3})R^{b3}, P(O)(OR^{a3})(OR^{a3}), P(O)(OR^{a3})R^{b3}, P(O)(NH₂)R^{b3}, P(O)(NH₂)(OR^{a3}), and B(OR^{a3})(OR^{a3}); wherein the C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{A2} are each optionally substituted with 1, 2, 3, or 4 substituents independently selected from R^{A3};
each R^{A3} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4} NR^{c4}R^{d4} NR^{c4}C(O)R^{b4}, NR^{c4}C(O)OR^{a4} NR^{c4}C(O)NR^{c4}R^{d4}, C(═NR^{c4})R^{b4}, C(═NOR^{a4})R^{b4}, C(═NR^{c4})NR^{c4}R^{d4} NR^{c4}C(═NR⁴)NR^{c4}R^{d4} NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{A3} are each optionally substituted with 1, 2, 3, or 4 substituents independently selected from R^{g1}; and wherein the C₁₋₆ alkyl, C₂₋₆ alkenyl, and C₂₋₆ alkynyl forming R^{A3} are each optionally substituted with 1, 2, 3, or 4 substituents independently selected from R^{g2};
each R^{Cy} is independently selected from C₁₋₆ alkyl, C₂₋₆ alkenyl, C₂₋₆ alkynyl, C₁₋₆ haloalkyl, C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene, 5-10 membered heteroaryl-C₁₋₃ alkylene, halo, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5} NR^{c5}R^{d5} NR^{c5}C(O)R^{b5}, NR^{c5}C(O)OR^{a5} NR^{c5}C(O)NR^{c5}R^{d5}, C(═NR^{c5})R^{b5}, C(═NOR^{a5})R^{b5}, C(═NR^{c5})NR^{c5}R^{d5} NR⁵C(═NR⁵)NR^{c5}R^{d5} NR^{c5}S(O)R^{d5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}; wherein the C₃₋₁₀ cycloalkyl, 4-12 membered heterocycloalkyl, C₆₋₁₀ aryl, 5-10 membered heteroaryl, C₃₋₁₀ cycloalkyl-C₁₋₃ alkylene, 4-12 membered heterocycloalkyl-C₁₋₃ alkylene, C₆₋₁₀ aryl-C₁₋₃ alkylene and 5-10 membered heteroaryl-C₁₋₃ alkylene forming R^{Cy} are each optionally substituted with 1, 2, 3, or 4 substituents independently selected from R^{Cy1}; and
wherein the C₁₋₆ alkyl, C₂₋₆ alkenyl, and C₂₋₆ alkynyl forming R^{Cy} are each optionally substituted with 1, 2, 3, or 4 substituents independently selected from R^{Cy2};
each R^{Cy1} is independently selected from C₁₋₆ alkyl, C₂₋₆ alkenyl, C₂₋₆ alkynyl, C₁₋₆ haloalkyl, and R^{Cy2}; wherein the C₁₋₆ alkyl, C₂₋₆ alkenyl, and C₂₋₆ alkynyl forming R^{Cy1} are each optionally substituted with 1, 2, 3, or 4 substituents independently selected from R^{Cy2};
each R^{Cy2} is independently selected from C₃₋₁₀ cycloalkyl, 4-10 membered heterocycloalkyl, C₆₋₁₀ aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene, 5-10 membered heteroaryl-C₁₋₃ alkylene, halo, CN, OR^{a6}, SR^{a6}, C(O)R^{b6}, C(O)NR^{c6}R^{d6}, C(O)OR^{a6}, NR^{c6}R^{d6} NR^{a6}C(O)R^{b6}NR^{c6}C(O)OR^{a6} NR^{c6}S(O)R^{b6}, NR^{c6}S(O)₂R^{b6}, NR^{c6}S(O)₂NR^{c6}R^{d6}, S(O)R^{b6}, S(O)NR^{c6}R^{d6} S(O)₂R^{b6}, and S(O)₂NR^{c6}R^{d6}; wherein the C₃₋₁₀ cycloalkyl, 4-10 membered heterocycloalkyl, C₆₋aryl, 5-10 membered heteroaryl, C₃₋₁₀ cycloalkyl-C₁₋₃ alkylene, 4-10 membered heterocycloalkyl-C₁₋₃ alkylene, C₆₋₁₀ aryl-C₁₋₃ alkylene and 5-10 membered heteroaryl-C₁₋₃ alkylene forming R^{Cy2} are each optionally substituted with 1, 2, 3, or 4 substituents independently selected from R^{g1};
each R^{a3}, R^{c3}, R^{d3}, R^{a4}, R^{c4}, R^{d4}, R^{a5}, R^{c5}, R^{d5}, R^{a6}, R^{c6}, R^{d6} and R^{a10} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a3}, R^{c3}, R^{d3}, R^{a4}, R^{c4}, R^{d4}, R^{a5}, R^{c5}, R^{d5}, R^{a6}, R^{c6}, R^{d6}, and R^{a10} are each optionally substituted with 1, 2, 3, or 4 substituents independently selected from R^{g1}; and wherein the C₁₋₆ alkyl, C₂₋₆ alkenyl, and C₂₋₆ alkynyl forming R^{a3}, R^{c3}, R^{d3}, R^{a4}, R^{c4}, R^{d4}, R^{a8}, R^{c5}, R^{d5}, R^{a6}, R^{c6}, R^{d6}, and R^{a10} are each optionally substituted with 1, 2, 3, or 4 substituents independently selected from R^{g2};
or any R^{c3} and R^{d3} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c4} and R^{d4} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c5} and R^{d5} attached to the same N atom, together with the N atom to which they are attached, form a 4-, 5-, 6- or 7-membered heterocycloalkyl group optionally substituted with 1, 2, 3, or 4 substituents independently selected from R^{g1};
or any R^{c6} and R^{d6} attached to the same N atom, together with the N atom to which they are attached, form a 4-, 5-, 6- or 7-membered heterocycloalkyl group optionally substituted with 1, 2, 3, or 4 substituents independently selected from R^{g1};
each R^{b3}, R^{b4}, R^{b5}, and R^{b6} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b3}, R^{b4}, R^{b5}, and R^{b6} are each optionally substituted with 1, 2, 3, or 4 substituents independently selected from R^{g1}; and wherein the C₁₋₆ alkyl, C₂₋₆ alkenyl, and C₂₋₆ alkynyl forming R^{b3}, R^{b4}, R^{b5}, and R^{b6} are each optionally substituted with 1, 2, 3, or 4 substituents independently selected from R^{g2};
each R^{c3}, R^{c4}, and R^{c5} is independently selected from H, CN, C₁₋₆ alkyl, C₁₋₆ haloalkyl, C₁₋₆ alkylthio, C₁₋₆ alkylsulfonyl, C₁₋₆ alkylcarbonyl, C₁₋₆ alkylaminosulfonyl, carbamyl, C₁₋₆ alkylcarbamyl, di(C₁₋₆ alkyl)carbamyl, aminosulfonyl, C₁₋₆ alkylaminosulfonyl and di(C₁₋₆ alkyl)aminosulfonyl;
each R^{g1} is independently selected from C₁₋₆ alkyl, C₂₋₆ alkenyl, C₂₋₆ alkynyl, and R^{g2} wherein the C₁₋₆ alkyl, C₂₋₆ alkenyl, and C₂₋₆ alkynyl are each optionally substituted with 1, 2, or 3 substituents independently selected from R^{g2};
each R^{g2} is independently selected from C₃₋₁₀ cycloalkyl, 4-10 membered heterocycloalkyl, C₆₋₁₀ aryl, 5-10 membered heteroaryl, halo, CN, OR^{a11}, SR^{a11}, C(O)R^{b11}, C(O)NR^{c11}R^{d11}, C(O)OR^{a11}, NR^{c11}R^{d11} NR^{c11}C(O)R^{b11}, NR^{c11}C(O)OR^{a11}, NR^{c11}S(O)R^{b11}, NR^{c11}S(O)₂R^{b11}, NR^{c11}S(O)₂NR^{c11}R^{d11} S(O)R^{b11}, S(O)NR^{c11}R^{d11}, S(O)₂R^{b11}, and S(O)₂NR^{c11}R^{d11}; wherein the C₃₋₁₀ cycloalkyl, 4-10 membered heterocycloalkyl, C₆₋₁₀ aryl, and 5-10 membered heteroaryl forming R^{g2} are each optionally substituted with 1, 2, 3, or 4 substituents independently selected from R^{h1};
each R^{a11}, R^{c11}, and R^{d11} is independently selected from H, C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{a11}, R^{c11}, and R^{d11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{a11}, R^{c11}, and R^{d11} are each optionally substituted with 1, 2, 3, or 4 substituents independently selected from R^{h2};
each R^{b11} is independently selected from C₁₋₆ alkyl, C₂₋₆ alkenyl, C₂₋₆ alkynyl, C₁₋₆ haloalkyl, C₃₋₁₀ cycloalkyl, 4-10 membered heterocycloalkyl, C₆₋₁₀ aryl and 5-10 membered heteroaryl; wherein the C₃₋₁₀ cycloalkyl, 4-10 membered heterocycloalkyl, C₆₋₁₀ aryl and 5-10 membered heteroaryl forming R^{b11} are each optionally substituted with 1, 2, 3, or 4 substituents independently selected from R^{h1}; and wherein the C₁₋₆ alkyl, C₂₋₆ alkenyl, and C₂₋₆ alkynyl forming R^{b11} are each optionally substituted with 1, 2, 3, or 4 substituents independently selected from R^{h2};
each R^{h1} is independently selected from C₁₋₆ alkyl, C₂₋₆ alkenyl, C₂₋₆ alkynyl, and R^{h2}, wherein the C₁₋₆ alkyl, C₂₋₆ alkenyl, and C₂₋₆ alkynyl are each optionally substituted with 1, 2, or 3 substituents independently selected from R^{h2}; and
each R^{h2} is independently selected from D, OH, NO₂, CN, halo, C₃₋₆ cycloalkyl, C₃₋₆ cycloalkyl-C₁₋₂ alkylene, C₁₋₆ alkoxy, C₁₋₆ haloalkoxy, C₁₋₃ alkoxy-C₁₋₃ alkyl, C₁₋₃ alkoxy-C₁₋₃ alkoxy, HO—C₁₋₃ alkoxy, HO—C₁₋₃ alkyl, cyano-C₁₋₃ alkyl, H₂N—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

102. The compound or pharmaceutically acceptable salt thereof of claim 1, wherein the compound has Formula (II-A): 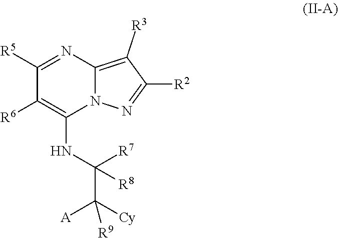
or a pharmaceutically acceptable salt thereof, wherein:
R², R³, R⁵, and R⁶ are each independently selected from H, D, and C₁₋₆ haloalkyl;
R⁷ and R⁸ are each independently selected from H and D;
R⁹ is selected from H and C₁₋₆ alkyl, wherein the C₁₋₆ alkyl forming R⁹ is optionally substituted with OR^{a10};
A is a ring selected from 4-12 membered heterocycloalkyl, wherein the 4-12 membered heterocycloalkyl forming A is optionally substituted with 1, 2, 3, 4, 5, or 6 substituents independently selected from R^{A};
Cy is phenyl optionally substituted with 1, 2, 3, 4, or 5 substituents independently selected from R^{Cy};
each R^{A} is selected from 5-10 membered heteroaryl, C₁₋₆ alkyl, OR^{a3}, NR^{c3}R^{d3}, C(O)R^{b3}, C(O)NR^{c3}R^{d3}, S(O)₂NR^{c3}R^{d3}, and C(═NR^{e3})NR^{c3}R^{d3}, wherein said 5-10 membered heteroaryl is optionally substituted with R^{A1} and said C₁₋₆ alkyl is optionally substituted with R^{A2};
each R^{A1} is independently selected from C₁₋₆ alkyl and R^{A2};
each R^{A2} is NR^{c3}R^{d3}, C(O)NR^{c3}R^{d3}, and OR^{a3};
each R^{Cy} is independently selected from halo;
each R^{a3}, R^{c3}, R^{d3}, and R^{a10} is independently selected from H and C₁₋₆ alkyl; and
each R^{c3} is independently selected from H, CN, and C₁₋₆ alkyl.

103. The compound or pharmaceutically acceptable salt thereof of claim 1, wherein the compound has Formula (II-A): 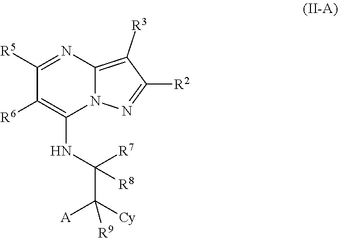
or a pharmaceutically acceptable salt thereof, wherein:
R², R³, R⁵, and R⁶ are each independently selected from H, D, and C₁₋₆ haloalkyl;
R⁷ and R⁸ are each independently selected from H and D;
R⁹ is selected from C₁₋₆ alkyl;
A is a ring selected from 4-12 membered heterocycloalkyl, wherein the 4-12 membered heterocycloalkyl forming A is optionally substituted with 1, 2, 3, 4, 5, or 6 substituents independently selected from R^{A};
Cy is phenyl optionally substituted with 1, 2, 3, 4, or 5 substituents independently selected from R^{Cy};
each R^{A} is selected from 5-10 membered heteroaryl, C(O)NR^{c3}R^{d3}, S(O)₂NR^{c3}R^{d3}, and C(═NR^{e3})NR^{c3}R^{d3}, wherein the 5-10 membered heteroaryl is optionally substituted with R^{A1};
each R^{A1} is independently selected from C₁₋₆ alkyl and R^{A2};
each R^{A2} is NR^{c3}R^{d3};
each R^{Cy} is independently selected from halo;
each R^{c3} and R^{d3} is independently selected from H and C₁₋₆ alkyl; and
each R^{c3} is independently selected from H, CN, and C₁₋₆ alkyl.

104. The compound or the pharmaceutically acceptable salt thereof of claim 1, wherein the compound is selected from:
3-(1-((2,5-bis(trifluoromethyl)pyrazolo[1,5-a]pyrimidin-7-yl)amino)-2-(4-fluorophenyl)butan-2-yl)azetidine-1-carboxamide;
4-(1-((2,5-bis(trifluoromethyl)pyrazolo[1,5-a]pyrimidin-7-yl)amino)-2-(4-fluorophenyl)propan-2-yl)piperidine-1-carboxamide;
3-(1-((2,5-bis(trifluoromethyl)pyrazolo[1,5-a]pyrimidin-7-yl)amino)-2-(4-fluorophenyl)propan-2-yl)pyrrolidine-1-carboxamide;
3-(1-((2,5-bis(trifluoromethyl)pyrazolo[1,5-a]pyrimidin-7-yl)amino)-2-(4-fluorophenyl)propan-2-yl)azetidine-1-carboxamide;
3-(1-((5-(difluoromethyl)-2-(trifluoromethyl)pyrazolo[1,5-a]pyrimidin-7-yl)amino)-2-phenylpropan-2-yl)azetidine-1-sulfonamide;
3-(1-((5-(difluoromethyl)-2-(trifluoromethyl)pyrazolo[1,5-a]pyrimidin-7-yl)amino)-2-phenylpropan-2-yl)azetidine-1-carboxamide;
N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine;
3-(1-((2,5-bis(trifluoromethyl)pyrazolo[1,5-a]pyrimidin-7-yl)amino)-2-phenylpropan-2-yl)azetidine-1-carboxamide;
N-(2-(azetidin-3-yl)-2-phenylpropyl)-2,5-bis(trifluoromethyl)pyrazolo[1,5-a]pyrimidin-7-amine;
6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one;
N-(2-(azetidin-3-yl)-2-(4-fluorophenyl)propyl)-2,5-bis(trifluoromethyl)pyrazolo[1,5-a]pyrimidin-7-amine; and
3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide,
or a pharmaceutically acceptable salt of any of the aforementioned.

105. The compound or the pharmaceutically acceptable salt thereof of claim 1, wherein the compound is selected from:
4-(2-((2,5-bis(trifluoromethyl)pyrazolo[1,5-a]pyrimidin-7-yl)amino)-1-(4-fluorophenyl)ethyl)piperidine-1-carboxamide;
3-(1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
2-(3-(1-((2,5-bis(trifluoromethyl)pyrazolo[1,5-a]pyrimidin-7-yl)amino)-2-(4-fluorophenyl)propan-2-yl)pyrrolidin-1-yl)acetamide;
3-(1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
3-(2-((2,5-bis(trifluoromethyl)pyrazolo[1,5-a]pyrimidin-7-yl)amino)-1-(4-fluorophenyl)ethyl)pyrrolidine-1-carboxamide;
3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide;
2-(2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol;
N-(2-(4-fluorophenyl)-2-(3-(pyridin-2-yl)azetidin-1-yl)ethyl)-2,5-bis(trifluoromethyl)pyrazolo[1,5-a]pyrimidin-7-amine;
2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one;
1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide;
N-(2-(4-fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine;
N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine;
6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide;
(3R)-1-(2-((2,5-bis(trifluoromethyl)pyrazolo[1,5-a]pyrimidin-7-yl)amino)-1-(4-fluorophenyl)ethyl)-3-methylpyrrolidin-3-ol;
((3S)-1-(2-((2,5-bis(trifluoromethyl)pyrazolo[1,5-a]pyrimidin-7-yl)amino)-1-(4-fluorophenyl)ethyl)pyrrolidin-3-yl)methanol;
2-(2-((2,5-bis(trifluoromethyl)pyrazolo[1,5-a]pyrimidin-7-yl)amino)-1-(4-fluorophenyl)ethyl)-2,5-diazaspiro[3.4]octan-6-one;
6-(2-((2,5-bis(trifluoromethyl)pyrazolo[1,5-a]pyrimidin-7-yl)amino)-1-(4-fluorophenyl)ethyl)-2,6-diazaspiro[3.4]octane-2-carboxamide;
2-(2-((2,5-bis(trifluoromethyl)pyrazolo[1,5-a]pyrimidin-7-yl)amino)-1-(4-fluorophenyl)ethyl)-2-azaspiro[3.3]heptan-5-ol;
1-(2-((2,5-bis(trifluoromethyl)pyrazolo[1,5-a]pyrimidin-7-yl)amino)-1-(4-fluorophenyl)ethyl)-3-methylazetidin-3-ol;
2-(2-((2,5-bis(trifluoromethyl)pyrazolo[1,5-a]pyrimidin-7-yl)amino)-1-(4-fluorophenyl)ethyl)-2,6-diazaspiro[3.4]octan-7-one;
2-(2-((2,5-bis(trifluoromethyl)pyrazolo[1,5-a]pyrimidin-7-yl)amino)-1-(4-fluorophenyl)ethyl)-2,5,7-triazaspiro[3.4]octan-6-one;
1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile;
1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one;
6-(2-((2,5-bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide;
6-(2-((2,5-bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide; and
2-(2-((2,5-bis(trifluoromethyl)pyrazolo[1,5-a]pyrimidin-7-yl)amino)-1-(4-fluorophenyl)ethyl)-7-oxa-2,5-diazaspiro[3.4]octan-6-one,
or a pharmaceutically acceptable salt of any of the aforementioned.

106. The compound or the pharmaceutically acceptable salt thereof of claim 1, wherein the compound is selected from:
(R)-3-(1-((2,5-bis(trifluoromethyl)pyrazolo[1,5-a]pyrimidin-7-yl)amino)-2-(4-fluorophenyl)butan-2-yl)azetidine-1-carboxamide;
(S)-3-(1-((2,5-bis(trifluoromethyl)pyrazolo[1,5-a]pyrimidin-7-yl)amino)-2-(4-fluorophenyl)butan-2-yl)azetidine-1-carboxamide;
(R)-4-(1-((2,5-bis(trifluoromethyl)pyrazolo[1,5-a]pyrimidin-7-yl)amino)-2-(4-fluorophenyl)propan-2-yl)piperidine-1-carboxamide;
(S)-4-(1-((2,5-bis(trifluoromethyl)pyrazolo[1,5-a]pyrimidin-7-yl)amino)-2-(4-fluorophenyl)propan-2-yl)piperidine-1-carboxamide;
(R*)-3-((R*)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R*)-3-((S*)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R)-3-((R)-1-((2,5-bis(trifluoromethyl)pyrazolo[1,5-a]pyrimidin-7-yl)amino)-2-(4-fluorophenyl)propan-2-yl)pyrrolidine-1-carboxamide;
(S)-3-((S)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R)-3-((S)-1-((2,5-bis(trifluoromethyl)pyrazolo[1,5-a]pyrimidin-7-yl)amino)-2-(4-fluorophenyl)propan-2-yl)pyrrolidine-1-carboxamide;
(S)-3-((R)-1-((2,5-bis(trifluoromethyl)pyrazolo[1,5-a]pyrimidin-7-yl)amino)-2-(4-fluorophenyl)propan-2-yl)pyrrolidine-1-carboxamide;
(R)-3-(1-((2,5-bis(trifluoromethyl)pyrazolo[1,5-a]pyrimidin-7-yl)amino)-2-(4-fluorophenyl)propan-2-yl)azetidine-1-carboxamide;
(S)-3-(1-((2,5-bis(trifluoromethyl)pyrazolo[1,5-a]pyrimidin-7-yl)amino)-2-(4-fluorophenyl)propan-2-yl)azetidine-1-carboxamide;
(R)-3-(1-((5-(difluoromethyl)-2-(trifluoromethyl)pyrazolo[1,5-a]pyrimidin-7-yl)amino)-2-phenylpropan-2-yl)azetidine-1-sulfonamide;
(S)-3-(1-((5-(difluoromethyl)-2-(trifluoromethyl)pyrazolo[1,5-a]pyrimidin-7-yl)amino)-2-phenylpropan-2-yl)azetidine-1-sulfonamide;
(R)—N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine;
(S)—N-(2-(1-(5-amino-1H-1,2,4-triazol-3-yl)azetidin-3-yl)-2-phenylpropyl)-2,5-bis(trifluoromethyl)pyrazolo[1,5-a]pyrimidin-7-amine;
(R)-3-(1-((2,5-bis(trifluoromethyl)pyrazolo[1,5-a]pyrimidin-7-yl)amino)-2-phenylpropan-2-yl)azetidine-1-carboxamide;
(S)-3-(1-((2,5-bis(trifluoromethyl)pyrazolo[1,5-a]pyrimidin-7-yl)amino)-2-phenylpropan-2-yl)azetidine-1-carboxamide;
(R)—N-(2-(azetidin-3-yl)-2-phenylpropyl)-2,5-bis(trifluoromethyl)pyrazolo[1,5-a]pyrimidin-7-amine;
(S)—N-(2-(azetidin-3-yl)-2-phenylpropyl)-2,5-bis(trifluoromethyl)pyrazolo[1,5-a]pyrimidin-7-amine;
(R)-6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one;
(S)-6-(3-(1-((2,5-bis(trifluoromethyl)pyrazolo[1,5-a]pyrimidin-7-yl)amino)-2-(4-fluorophenyl)propan-2-yl)azetidin-1-yl)-2-methylpyridazin-3(2H)-one;
(R)—N-(2-(azetidin-3-yl)-2-(4-fluorophenyl)propyl)-2,5-bis(trifluoromethyl)pyrazolo[1,5-a]pyrimidin-7-amine;
(S)—N-(2-(azetidin-3-yl)-2-(4-fluorophenyl)propyl)-2,5-bis(trifluoromethyl)pyrazolo[1,5-a]pyrimidin-7-amine;
(R)-3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide; and
(S)-3-(1-((2,5-bis(trifluoromethyl)pyrazolo[1,5-a]pyrimidin-7-yl)amino)-2-(4-fluorophenyl)propan-2-yl)-N-cyanoazetidine-1-carboximidamide,
or a pharmaceutically acceptable salt of any of the aforementioned.

107. The compound or the pharmaceutically acceptable salt thereof of claim 1, wherein the compound is selected from:
(R)-4-(2-((2,5-bis(trifluoromethyl)pyrazolo[1,5-a]pyrimidin-7-yl)amino)-1-(4-fluorophenyl)ethyl)piperidine-1-carboxamide;
(S)-4-(2-((2,5-bis(trifluoromethyl)pyrazolo[1,5-a]pyrimidin-7-yl)amino)-1-(4-fluorophenyl)ethyl)piperidine-1-carboxamide;
(R*)-3-((R*)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(R)-3-((R)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(S)-3-((S)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
R*)-3-((S*)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(R)-3-((S)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
(S)-3-((R)-1-((2,5-bis(trifluoromethyl)pyrazolo[1,5-a]pyrimidin-7-yl)amino)-2-(4-fluorophenyl)propan-2-yl)-N-(2-hydroxyethyl)pyrrolidine-1-carboxamide;
2-((R*)-3-((R*)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((R)-3-((R)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((S)-3-((S)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((R*)-3-((S*)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((R)-3-((S)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
2-((S)-3-((R)-1-((2,5-bis(trifluoromethyl)pyrazolo[1,5-a]pyrimidin-7-yl)amino)-2-(4-fluorophenyl)propan-2-yl)pyrrolidin-1-yl)acetamide;
(R*)-3-((S*)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R)-3-((S)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(S)-3-((R)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R*)-3-((R*)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R)-3-((R)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(S)-3-((S)-1-((2,5-bis(trifluoromethyl)pyrazolo[1,5-a]pyrimidin-7-yl)amino)-2-(4-fluorophenyl)-3-hydroxypropan-2-yl)pyrrolidine-1-carboxamide;
(R*)-3-((R*)-2-((2,5-bis(trifluoromethyl)pyrazolo[1,5-a]pyrimidin-7-yl)amino)-1-(4-fluorophenyl)ethyl)pyrrolidine-1-carboxamide;
(R)-3-((R)-2-((2,5-bis(trifluoromethyl)pyrazolo[1,5-a]pyrimidin-7-yl)amino)-1-(4-fluorophenyl)ethyl)pyrrolidine-1-carboxamide;
(S)-3-((S)-2-((2,5-bis(trifluoromethyl)pyrazolo[1,5-a]pyrimidin-7-yl)amino)-1-(4-fluorophenyl)ethyl)pyrrolidine-1-carboxamide;
(R*)-3-((S*)-2-((2,5-bis(trifluoromethyl)pyrazolo[1,5-a]pyrimidin-7-yl)amino)-1-(4-fluorophenyl)ethyl)pyrrolidine-1-carboxamide;
(R)-3-((S)-2-((2,5-bis(trifluoromethyl)pyrazolo[1,5-a]pyrimidin-7-yl)amino)-1-(4-fluorophenyl)ethyl)pyrrolidine-1-carboxamide;
(S)-3-((R)-2-((2,5-bis(trifluoromethyl)pyrazolo[1,5-a]pyrimidin-7-yl)amino)-1-(4-fluorophenyl)ethyl)pyrrolidine-1-carboxamide;
(R)-3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide;
(S)-3-(1-((2,5-bis(trifluoromethyl)pyrazolo[1,5-a]pyrimidin-7-yl)amino)-2-(4-fluorophenyl)-3-hydroxypropan-2-yl)azetidine-1-carboxamide;
(1R*,4R*)-2-((R*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4R)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4S)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R*,4R*)-2-((S*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4R)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4S)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S*,4R*)-2-((S*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4R)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4S)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S*,4R*)-2-((R*)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1S,4R)-2-((R)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(1R,4S)-2-((S)-2-((2,5-bis(trifluoromethyl)pyrazolo[1,5-a]pyrimidin-7-yl)amino)-1-(4-fluorophenyl)ethyl)-2-azabicyclo[2.2.1]heptan-5-ol;
(R)-2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol;
(S)-2-(2-((2,5-bis(trifluoromethyl)pyrazolo[1,5-a]pyrimidin-7-yl)amino)-1-(4-fluorophenyl)ethyl)-2-azaspiro[3.5]nonan-7-ol;
(R)—N-(2-(4-fluorophenyl)-2-(3-(pyridin-2-yl)azetidin-1-yl)ethyl)-2,5-bis(trifluoromethyl)pyrazolo[1,5-a]pyrimidin-7-amine;
(S)—N-(2-(4-fluorophenyl)-2-(3-(pyridin-2-yl)azetidin-1-yl)ethyl)-2,5-bis(trifluoromethyl)pyrazolo[1,5-a]pyrimidin-7-amine;
(R)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one;
(S)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.5]nonan-6-one;
(R)-1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide;
(S)-1-(2-((2,5-bis(trifluoromethyl)pyrazolo[1,5-a]pyrimidin-7-yl)amino)-1-(4-fluorophenyl)ethyl)-N-(methyl-d₃)azetidine-3-carboxamide;
(R)—N-(2-(4-fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine;
(S)—N-(2-(4-fluorophenyl)-2-(3-((1-methyl-1H-1,2,3-triazol-4-yl)amino)azetidin-1-yl)ethyl)-2,5-bis(trifluoromethyl)pyrazolo[1,5-a]pyrimidin-7-amine;
(R)—N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine;
(S)—N-(2-(4-fluorophenyl)-2-(3-((1-methyl-1H-imidazol-2-yl)amino)azetidin-1-yl)ethyl)-2,5-bis(trifluoromethyl)pyrazolo[1,5-a]pyrimidin-7-amine;
(R)-6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide;
(S)-6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carboxamide;
(R)-1-((R)-2-((2,5-bis(trifluoromethyl)pyrazolo[1,5-a]pyrimidin-7-yl)amino)-1-(4-fluorophenyl)ethyl)-3-methylpyrrolidin-3-ol;
(R)-1-((S)-2-((2,5-bis(trifluoromethyl)pyrazolo[1,5-a]pyrimidin-7-yl)amino)-1-(4-fluorophenyl)ethyl)-3-methylpyrrolidin-3-ol;
((S)-1-((R)-2-((2,5-bis(trifluoromethyl)pyrazolo[1,5-a]pyrimidin-7-yl)amino)-1-(4-fluorophenyl)ethyl)pyrrolidin-3-yl)methanol;
((S)-1-((S)-2-((2,5-bis(trifluoromethyl)pyrazolo[1,5-a]pyrimidin-7-yl)amino)-1-(4-fluorophenyl)ethyl)pyrrolidin-3-yl)methanol;
(R)-2-(2-((2,5-bis(trifluoromethyl)pyrazolo[1,5-a]pyrimidin-7-yl)amino)-1-(4-fluorophenyl)ethyl)-2,5-diazaspiro[3.4]octan-6-one;
(S)-2-(2-((2,5-bis(trifluoromethyl)pyrazolo[1,5-a]pyrimidin-7-yl)amino)-1-(4-fluorophenyl)ethyl)-2,5-diazaspiro[3.4]octan-6-one;
(R)-6-(2-((2,5-bis(trifluoromethyl)pyrazolo[1,5-a]pyrimidin-7-yl)amino)-1-(4-fluorophenyl)ethyl)-2,6-diazaspiro[3.4]octane-2-carboxamide;
(S)-6-(2-((2,5-bis(trifluoromethyl)pyrazolo[1,5-a]pyrimidin-7-yl)amino)-1-(4-fluorophenyl)ethyl)-2,6-diazaspiro[3.4]octane-2-carboxamide;
(S*)-2-((R*)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(S)-2-((R)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(R)-2-((S)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(S*)-2-((S*)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(S)-2-((S)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(R)-2-((R)-2-((2,5-bis(trifluoromethyl)pyrazolo[1,5-a]pyrimidin-7-yl)amino)-1-(4-fluorophenyl)ethyl)-2-azaspiro[3.3]heptan-5-ol;
(R)-1-(2-((2,5-bis(trifluoromethyl)pyrazolo[1,5-a]pyrimidin-7-yl)amino)-1-(4-fluorophenyl)ethyl)-3-methylazetidin-3-ol;
(S)-1-(2-((2,5-bis(trifluoromethyl)pyrazolo[1,5-a]pyrimidin-7-yl)amino)-1-(4-fluorophenyl)ethyl)-3-methylazetidin-3-ol;
(R)-2-(2-((2,5-bis(trifluoromethyl)pyrazolo[1,5-a]pyrimidin-7-yl)amino)-1-(4-fluorophenyl)ethyl)-2,6-diazaspiro[3.4]octan-7-one;
(S)-2-(2-((2,5-bis(trifluoromethyl)pyrazolo[1,5-a]pyrimidin-7-yl)amino)-1-(4-fluorophenyl)ethyl)-2,6-diazaspiro[3.4]octan-7-one;
(R)-2-(2-((2,5-bis(trifluoromethyl)pyrazolo[1,5-a]pyrimidin-7-yl)amino)-1-(4-fluorophenyl)ethyl)-2,5,7-triazaspiro[3.4]octan-6-one;
(S)-2-(2-((2,5-bis(trifluoromethyl)pyrazolo[1,5-a]pyrimidin-7-yl)amino)-1-(4-fluorophenyl)ethyl)-2,5,7-triazaspiro[3.4]octan-6-one;
(R)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile;
(S)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptane-2-carbonyl)cyclopropane-1-carbonitrile;
(R)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one;
(S)-1-(6-(2-((2,5-bis(trifluoromethyl)pyrazolo[1,5-a]pyrimidin-7-yl)amino)-1-(4-fluorophenyl)ethyl)-2,6-diazaspiro[3.3]heptan-2-yl)-2-hydroxyethan-1-one;
(R)-6-(2-((2,5-bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide;
(S)-6-(2-((2,5-bis(trifluoromethyl)pyrazolo[1,5-a]pyrimidin-7-yl)amino)-1-(4-fluorophenyl)ethyl)-N-(methyl-d₃)-2,6-diazaspiro[3.3]heptane-2-carboxamide;
(R)-6-(2-((2,5-bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide;
(S)-6-(2-((2,5-bis(trifluoromethyl)pyrazolo[1,5-a]pyrimidin-7-yl)amino)-1-(4-fluorophenyl)ethyl)-N-(2,2,2-trifluoroethyl)-2,6-diazaspiro[3.3]heptane-2-carboxamide;
(R)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.4]octan-6-one; and
(S)-2-(2-((2,5-bis(trifluoromethyl)pyrazolo[1,5-a]pyrimidin-7-yl)amino)-1-(4-fluorophenyl)ethyl)-7-oxa-2,5-diazaspiro[3.4]octan-6-one,
or a pharmaceutically acceptable salt of any of the aforementioned.

108. A pharmaceutical composition comprising a compound or pharmaceutically acceptable salt thereof of claim 1 and a pharmaceutically acceptable carrier or excipient.

109. A method of treating an MRGPRX2 dependent condition in a patient comprising administering to the patient a therapeutically effective amount of a compound or pharmaceutically acceptable salt thereof of claim 1.

110. The method of claim 109, wherein the MRGPRX2 dependent condition is an itch associated condition, a pain associated condition, a pseudo-allergic reaction, an autoimmune or inflammatory disorder, or a cancer or tumor associated condition.

111. The method of claim 109, wherein the MRGPRX2 dependent condition is an itch associated condition.

112. The method of claim 111, wherein the MRGPRX2 dependent condition is an itch associated condition selected from the group consisting of chronic itch; senile itch; contact dermatitis; allergic blepharitis; anaphylaxis; anaphylactoid drug reactions; anaphylactic shock; anemia; atopic dermatitis; bullous pemphigoid; candidiasis; chicken pox; end-stage renal failure; hemodialysis; cholestatic pruritus; chronic spontaneous urticaria; chronic inducible urticaria; contact dermatitis, dermatitis herpetiformis; diabetes; drug allergy, dry skin; dyshidrotic dermatitis; ectopic eczema; eosinophilic fasciitis; epidermolysis bullosa; erythrasma; food allergy; folliculitis; fungal skin infection; hemorrhoids; herpes; HIV infection; hodgkin's disease; hyperthyroidism; iodinated contrast dye allergy; iron deficiency anemia; kidney disease; leukemia, porphyrias; lymphoma; mast cell activation syndrome, malignancy; mastocystosis; multiple myeloma; neurodermatitis; onchocerciasis; Paget's disease; pediculosis; polycythemia rubra vera; prurigo nodularis; lichen planus; lichen sclerosis; pruritus ani; pseudo-allergic reactions; pseudorabies; psoriasis; rectal prolapse; sarcoidosis granulomas; scabies; schistosomiasis; scleroderma, severe stress, stasia dermatitis; swimmer's itch; thyroid disease; tinea cruris; uremic pruritus; rosacea; cutaneous amyloidosis; scleroderma; acne; wound healing; burn healing; ocular itch; and urticaria.

113. The method of claim 109, wherein the MRGPRX2 dependent condition is a pain associated condition.

114. The method of claim 113, wherein the MRGPRX2 dependent condition is a pain associated condition selected from the group consisting of acute pain, advanced prostate cancer, AIDS-related pain, ankylosing spondylitis, arachnoiditis, arthritis, arthrofibrosis, ataxic cerebral palsy, autoimmune atrophic gastritis, avascular necrosis, back pain, Behcet's disease (syndrome), burning mouth syndrome, bursitis, cancer pain, carpal tunnel, cauda equina syndrome, central pain syndrome, cerebral palsy, cervical stenosis, Charcot-Marie-Tooth (CMT) disease, chronic fatigue syndrome (CFS), chronic functional abdominal pain (CFAP), chronic pain, chronic pancreatitis, chronic pelvic pain syndrome, collapsed lung (pneumothorax), complex regional pain syndrome (CRPS), reflex sympathetic dystrophy syndrome (RDS), corneal neuropathic pain, Crohn's disease, degenerative disc disease, dental pain, Dercum's disease, dermatomyositis, diabetic peripheral neuropathy (DPN), dystonia, Ehlers-Danlos syndrome (EDS), endometriosis, eosinophilia-myalgia syndrome (EMS), erythromelalgia, fibromyalgia, gout, headaches, herniated disc, hydrocephalus, intercostal neuralgia, interstitial cystitis, irritable bowel syndrome (IBS), juvenile dermatositis (dermatomyositis), knee injury, leg pain, loin pain-haematuria syndrome, lupus, Lyme disease, medullary sponge kidney (MSK), meralgia paresthetica, mesothelioma, migraine, musculoskeletal pain, myofascial pain, myositis, neck pain, neuropathic pain, occipital neuralgia, osteoarthritis, Paget's disease, pain crisis in sickle cell disease; Parsonage-Turner syndrome, pelvic pain, periodontitis pain, peripheral neuropathy, phantom limb pain, pinched nerve, polycystic kidney disease, polymyalgia rheumatica, polymyositis, porphyria, post herniorrhaphy pain syndrome, post mastectomy pain, postoperative pain, pain syndrome, post stroke pain, post thoracotomy pain syndrome, postherpetic neuralgia (shingles), post-polio syndrome, primary lateral sclerosis, psoriatic arthritis, pudendal neuralgia, radiculopathy, raynaud's disease, rheumatoid arthritis (RA), sacroiliac joint dysfunction, sarcoidosis, Scheuermann's kyphosis disease, sciatica, scoliosis, shingles (herpes zoster), Sjögren's syndrome, spasmodic torticollis, sphincter of oddi dysfunction, spinal cerebellum ataxia (SCA ataxia), spinal cord injury, spinal stenosis, syringomyelia, Tarlov cysts, transverse myelitis, trigeminal neuralgia, neuropathic pain, ulcerative colitis, vascular pain and vulvodynia.

115. The method of claim 109, wherein the MRGPRX2 dependent condition is a pseudo-allergic reaction.

116. The method of claim 115, wherein the MRGPRX2 dependent condition is a pseudo-allergic reaction caused by a secretagogue, cationic peptidergic drug, anionic peptidergic drug, neutral peptidergic drug, non-steroidal antiinflammatory drugs, neuropeptides, antimicrobial peptides, opioids, neuromuscular blocking agents, antidepressant agents, antipsychotic agents, antihistamine agents, antineoplastic agents, fluoroquinolone and non-fluoroquinolone antibiotics and tyrosine-kinase inhibitors.

117. The method of claim 115, wherein the MRGPRX2 dependent condition is a pseudo-allergic reaction caused by MCD peptide, substance P, VIP, PACAP, dynorphin, somatostatin, Compound 48/80, cortistatin-14, mastoparan, melittin, a cathelicidin peptide, ciprofloxacin, vancomycin, leuprolide, goserelin, histrelin, triptorelin, cetrorelix, ganirelix, degarelix, octreotide, lanreotide, pasireotide, sermorelin, tesamorelin, icatibant, glatiramer acetate, teriparatide, pramlintide, bleomycin, exenatide, glucagon, liraglutide, enfuvirtide, colistimethate, succinylcholine, tubocurarine, atracurium, mivacurium, or rocuronium.

118. The method of claim 109, wherein the MRGPRX2 dependent condition is an autoimmune or inflammatory disorder.

119. The method of claim 118, wherein the MRGPRX2 dependent condition is an autoimmune or inflammatory disorder selected from as chronic inflammation, mast cell activation syndrome, multiple sclerosis, Steven Johnson's syndrome, toxic epidermal necrolysis, appendicitis, bursitis, cutaneous lupus, colitis, cystitis, dermatitis, phlebitis, reflex sympathetic dystrophy/complex regional pain syndrome (RSD/CRPS), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosinophilia, eosinophilic leukemia, eosinophilic esophagitis, eosinophilic gastritis, eosinophilic duodenitis, eosinophilic gastroenteritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, schwannomatosis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, biliary atresia, acute and chronic hepatitis B and C virus, liver tumors and cancers, alcoholic liver disease, polycystic liver disease, liver cholangiocarcinoma, primary sclerosing cholangitis, primary biliary cholangitis, neuromyelitis optica spectrum disorder, cardiovascular disease, inflammation induced by bacterial or viral infection, inflammation associated with SARS-COV-2 infection or its variants or coronavirus disease 2019 (COVID-19), acute respiratory distress syndrome, pneumonia, long/long-term/chronic COVID, postacute sequelae of COVID-19 (PASC), myalgic encephalomyelitis, chronic fatigue syndrome and vasculitis

120. The method of claim 109, wherein the MRGPRX2 dependent condition is a cancer or tumor associated condition.

121. The method of claim 120, wherein the MRGPRX2 dependent condition is a cancer or tumor associated condition selected from as adenoid cystic carcinoma, adrenal gland tumor, amyloidosis, anal cancer, appendix cancer, astrocytoma, ataxia-telangiectasia, Beckwith-Wiedemann syndrome, cholangiocarcinoma, Birt-Hogg-Dube syndrome, bone cancer, brain stem glioma, brain tumor, breast cancer (inflammatory, metastatic, male), prostrate, basal cell, melanoma, colon, colorectal, bladder, kidney cancer, lacrimal gland cancer, laryngeal and hypopharyngeal cancer, lung cancer (non-small cell, small cell), leukemia (acute lymphoblastic, acute lymphocytic, acute myeloid, B cell prolymphocytic, chronic lymphocytic, chronic myeloid, chronic T cell lymphocytic, eosinophilic), liver cancer, Li-Fraumeni syndrome, lymphoma (Hodgkin and non-Hodgkin), lynch syndrome, mastocytosis, medulloblastoma, meningioma, mesothelioma, multiple endocrine neoplasia, multiple myeloma, MUTYH-associated polyposis, myelodysplastic syndrome, nasal cavity and paranasal sinus cancer, neuroblastoma, neuroendocrine tumors, neurofibromatosis, penile cancer, parathyroid cancer, ovarian fallopian tube and peritoneal cancer, osteosarcoma, pituitary gland tumor, pleuropulmonary blastoma, oral and oropharyngeal, thyroid, uterine, pancreatic, carney complex, brain and spinal cord cancer, cervical cancer, Cowden syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor, germ cell tumor, gestational trophoblastic disease, head and neck cancer, hereditary breast and ovarian cancer, hereditary diffuse gastric cancer, hereditary, leiomyomatosis and renal cell cancer, hereditary pancreatitis, hereditary papillary renal carcinoma, hereditary mixed polyposis syndrome, HIV/AIDS related cancers, retinoblastoma, rhabdomyosarcoma, salivary gland cancer, Kaposi sarcoma, small bowel cancer, stomach cancer, testicular cancer, thymoma and thymic carcinoma, thyroid cancer, vaginal cancer, culver cancer, Wermer's syndroeroderma pigmentosum.

122. The method of claim 109, wherein the MRGPRX2 dependent condition is selected from abdominal aortic aneurysms, acute contact dermatitis, allergic rhinitis, amyotrophic lateral sclerosis, asthma, atopic dermatitis, autism, cancer, chronic inducible urticaria, chronic itch, chronic obstructive pulmonary disease, chronic spontaneous urticaria, cold urticaria, contact urticaria, coronary artery disease, cough, Crohn's disease, deep vein thrombosis, drug-induced anaphylactic reactions, endometriosis, fibromyalgia, geographic atrophy, idiopathic chronic cough, idiopathic pulmonary fibrosis, inflammatory pain, interstitial cystitis, irritable bowel syndrome, mast cell activation syndrome, mastocytosis, metabolic syndrome, migraine, multiple sclerosis, nasal polyps, neurodermatitis, neuropathic itch, neuropathic pain, obesity, oesophageal reflux, osteoarthritis, periodontitis, prurigo nodularis, pruritus, pseudo-anaphylaxis, psoriasis, rheumatoid arthritis, rosacea, seborrheic dermatitis, sickle cell disease, ulcerative colitis, and ulcers.

123. The method of claim 109, wherein the MRGPRX2 dependent condition is selected from autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, graft-versus-host disease (GvHD), Guillain-Barre syndrome, Hashimoto's encephalitis, Hashimoto's thyroiditis, ankylosing spondylitis, psoriasis, Sjögren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

124. The method of claim 109, wherein the MRGPRX2 dependent condition is selected from acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticaria, axonal or neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis (GPA) (formerly called Wegener's granulomatosis), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, Immunoregulatory lipoproteins, inclusion body myositis, interstitial cystitis, juvenile arthritis, juvenile diabetes (type 1 diabetes), juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, chronic, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, subacute bacterial endocarditis (SBE), Susac's syndrome, sympathetic ophthalmia, takayasu's arteritis, temporal arteritis/giant cell arteritis, thrombocytopenic purpura (TTP), tolosa-hunt syndrome, transverse myelitis, type 1 diabetes, ulcerative colitis, undifferentiated connective tissue disease (UCTD), uveitis, vasculitis, vesiculobullous dermatosis, vitiligo, and Wegener's granulomatosis (i.e., granulomatosis with polyangiitis (GPA)).
